(19) United States
(12) Patent Application Publication
(10) Pub. No.: US 2025/0154278 A1
(43) Pub. Date: May 15, 2025

(54) OPTIMIZED ANTIBODIES TARGETING TROP2 AND USES THEREOF

(71) Applicant: JANUX THERAPEUTICS, INC., San Diego, CA (US)

(72) Inventors: David CAMPBELL, San Diego, CA (US); Thomas R. DIRAIMONDO, San Diego, CA (US)

(21) Appl. No.: 18/840,196

(22) PCT Filed: Feb. 22, 2023
(86) PCT No.: PCT/US2023/063075

§ 371 Date: Aug. 21, 2024

Related U.S. Application Data
(60) Provisional application No. 63/485,687, filed on Feb. 17, 2023.
(60) Provisional application No. 63/313,167, filed on Feb. 23, 2022.

(51) Int. Cl. C07K 16/30; C07K 16/28

(52) U.S. Cl. CPC C07K 16/30; C07K 16/2809; C07K 2317/31; C07K 2317/622; C07K 2317/732; C07K 2317/92; C07K 2317/94

(57) ABSTRACT

Disclosed herein are recombinant antibodies or antigen binding fragments thereof that comprise a tumor-associated calcium signal transducer 2 (TROP2) binding domain that has been optimized for binding and kinetic properties. In some embodiments, the recombinant antibodies or antigen binding fragments thereof further comprise a CD3 binding domain.

### CROSS-REFERENCE

The present application claims the benefit of U.S. Provisional Application No. 63/313,167 filed Feb. 23, 2022, and U.S. Provisional Application No. 63,485,687 filed Feb. 17, 2023, each of which are incorporated herein by reference in their entirety.

### SEQUENCE LISTING

The instant application contains a Sequence Listing which has been submitted electronically in XML format and is hereby incorporated by reference in its entirety. Said XML copy, created on Feb. 6, 2023, is named 52426-740_601SL.xml and is 208,000 bytes in size.

### SUMMARY

Disclosed herein are recombinant antibodies or antigen binding fragments that comprise a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1; wherein CDR2-L comprises the amino acid sequence of SEQ ID NO: 2; and wherein CDR3-L comprises an amino acid sequence of X₁X₂HYX₃X₄X₅X₆X₇; wherein X₁ is Q, S, T, D, N, E, H, R, K, or A; X₂ is Q, S, T, D, N, E, H, K, R, or A; X₃ is I, G, P, V, L, M, S, T, or A; X₄ is T, G, S, M, H, N, Q, or A; X₅ is P, G, V, L, I, M, S, T, or A; X₆ is L, G, P, V, I, M, S, T, or A; and X₇ is T, G, S, M, H, N, Q, or A; wherein CDR1-H comprises the amino acid sequence of SEQ ID NO: 13; wherein CDR2-H comprises the amino acid sequence of SEQ ID NO: 14; and wherein CDR3-H comprises an amino acid sequence of AX₈X₉GX₁₀X₁₁X₁₂X₁₃YWX₁₄X₁₅X₁₆X₁₇; wherein X₈ is R, S, T, Q, D, E, N, H, K, or A; X₉ is G, P, V, L, I M, S, T, or A; X₁₀ is F, Y, W, V, L, I, G, or A; X₁₁ is G, P, V, L, I, M, S, T, or A; X₁₂ is S, G, T, M, N, Q, H, or A; X₁₃ is S, G, T, M, N, Q, H, or A; X₁₄ is Y, F, W, V, L, I, G, or A; X₁₅ is F, Y, W, V, L, I, G, or A; X₁₆ is D, Q, N, E, S, T, H, K, R, or A; and X₁₇ is V, G, P, L, I, M, S, T, or A; and wherein when CDR3-H comprises the amino acid sequence of SEQ ID NO: 15, then CDR3-L does not comprise the amino acid sequence of SEQ ID NO: 3. In some embodiments, X₁ is Q, N, D, E, or A; X₂ is Q, N, D, E, or A; X₃ is I, V, L, or A; X₄ is T, S, or A; X₅ is P, G, or A; X₆ is L, V, I, or A; X₇ is T, S, or A; X₈ is R, K, or A; X₉ is G, S, T, or A; X₁₀ is F, Y, or A; X₁₁ is G, S, T, or A; X₁₂ is S, G, T, or A; X₁₃ is S, G, T, or A; X₁₄ is Y, W, F, or A; X₁₅ is F, Y, W, or A; X₁₆ is D, E, Q, N, or A; and X₁₇ is V, L, I, or A. In some embodiments, X₁ is Q; and X₆ is L. In some embodiments, X₈ is R; X₁₀ is F; X₁₁ is G; X₁₄ is Y; X₁₅ is F; and X₁₆ is D.

In some embodiments, X₁ is Q, S, T, D, N, E, or A; X₂ is Q, S, T, D, N, E, or A; X₃ is I, G, P, V, L, M, or A; X₄ is T, G, S, M, H, N, Q, or A; X₅ is P, G, V, L, I, M, or A; X₆ is L, G, P, V, I, M, or A; X₇ is T, G, S, M, H, N, Q, or A; X₈ is R, H, K, or A; X₉ is G, P, V, L, I, M, S, T, or A; X₁₀ is F, Y, W, V, L, I, or A; X₁ is G, P, V, L, I, M, S, T, or A; X₁₂ is S, G, T, M, N, Q, or A; X₁₃ is S, G, T, M, N, Q, or A; X₁₄ is Y, F, W, V, L, I, or A; X₅ is F, Y, W, V, L, I, or A; X₁₁ is D, Q, N, E, S, T, or A; and X₁₇ is V, G, P, L, I, M, or A. In some embodiments, X₁ is Q, N, or A; X₂ is Q, N, or A; X₃ is I, V, L, or A; X₄ is T, S, or A; X₅ is P, G, or A; X₆ is L, V, I, or A; X₇ is T, S, or A; X₈ is R, K, or A; X₉ is G, V, S, T, or A; X₁₀ is F, Y, or A; X₁₁ is G, V, S, T, or A; X₁₂ is S, G, T, or A; X₁₃ is S, G, T, or A; X₁ is Y, W, or A; X₁₅ is F, Y, or A; X₁₆ is D, E, or A; and X₁₇ is V, G, L, I, or A. In some embodiments, X₁ is Q; and X₆ is L. In some embodiments, X₈ is R; X₁₀ is F; X₁₁ is G; X₁₄ is Y; X₁₅ is F; and X₁₆ is D.

In some embodiments, CDR3-L comprises an amino acid selected from SEQ ID NOs: 3-5, 8-12. In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11 and CDR1-H: SEQ ID NO: 13, CDR2-H: SEQ ID NO: 14, CDR3-H: SEQ ID NO: 15; and CDR1-L: SEQ ID NO: 1, CDR2-L: SEQ ID NO: 2, CDR3-L: SEQ ID NO: 12 and CDR1-H: SEQ ID NO: 13, CDR2-H: SEQ ID NO: 14, CDR3-H: SEQ ID NO: 15. In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 5 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10 and CDR1-H: SEQ ID NO: 13, CDR2-H: SEQ ID NO: 14, CDR3-H: SEQ ID NO: 15; and CDR1-L: SEQ ID NO: 1, CDR2-L: SEQ ID NO: 2, CDR3-L: SEQ ID NO: 12 and CDR1-H: SEQ ID NO: 13, CDR2-H: SEQ ID NO: 14, CDR3-H: SEQ ID NO: 15.

In some embodiments, CDR3-H comprises an amino acid selected from SEQ ID NOs: 16-17, 19-22, 25-28. In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14; CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28. In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO:21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

Disclosed herein are recombinant antibodies or antigen binding fragments that comprise a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 6 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 7 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 12 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 18; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14, CDR3-H: SEQ ID NO: 21; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14; CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 23; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 24; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

In some embodiments, the TROP2 binding domain comprises a Fab, Fab′, (Fab′)₂ or a single chain variable fragment (scFv). In some embodiments, the TROP2 binding domain is a Fab. In some embodiments, the immunoglobulin light chain comprises a variable domain of an immunoglobulin kappa (IgK) or immunoglobulin lambda (IgL) light chain. In some embodiments, the immunoglobulin heavy chain comprises a variable domain of an IgG1, IgG2, IgG3, or IgG4 heavy chain. In some embodiments, the immunoglobulin light chain comprises an amino acid sequence with at least 90%, 95%, 96%, 97%, 98%, 99%, or 100% identity to any one of SEQ ID NOs: 31, 33, 35, 37, 39, 41, 43, 45, 47, 49, 51, 53, 55, 57, 59, 61, 63, 65, 67, 69, 71, or 73. In some embodiments, the immunoglobulin heavy chain comprises an amino acid sequence with at least 90%, 95%, 96%, 97%, 98%, 99%, or 100% identity to any one of SEQ ID NOs: 32, 34, 36, 38, 40, 42, 44, 46, 48, 50, 52, 54, 56, 58, 60, 62, 64, 66, 68, 70, 72, or 74. In some embodiments, the immunoglobulin light chain comprises an amino acid sequence with at least 90%, 95%, 96%, 97%, 98%, 99%, or 100% identity to SEQ ID NO: 31 and the immunoglobulin heavy chain comprises an amino acid sequence with at least 90%, 95%, 96%, 97%, 98%, 99%, or 100% identity to SEQ ID NO: 32. In some embodiments, the immunoglobulin light chain comprises an amino acid sequence with at least 90%, 95%, 96%, 97%, 98%, 99%, or 100% identity to SEQ ID NO: 33 and the immunoglobulin heavy chain comprises an amino acid sequence with at least 90%, 95%, 96%, 97%, 98%, 99%, or 100% identity to SEQ ID NO: 34. In some embodiments, the immunoglobulin light chain comprises an amino acid sequence with at least 90%, 95%, 96%, 97%, 98%, 99%, or 100% identity to SEQ ID NO: 35 and the immunoglobulin heavy chain comprises an amino acid sequence with at least 90%, 95%, 96%, 97%, 98%, 99%, or 100% identity to SEQ ID NO: 36. In some embodiments, the immunoglobulin light chain comprises an amino acid sequence with at least 90%, 95%, 96%, 97%, 98%, 99%, or 100% identity to SEQ ID NO: 37 and the immunoglobulin heavy chain comprises an amino acid sequence with at least 90%, 95%, 96%, 97%, 98%, 99%, or 100% identity to SEQ ID NO: 38. In some embodiments, the immunoglobulin light chain comprises an amino acid sequence with at least 90%, 95%, 96%, 97%, 98%, 99%, or 100% identity to SEQ ID NO: 39 and the immunoglobulin heavy chain comprises an amino acid sequence with at least 90%, 95%, 96%, 97%, 98%, 99%, or 100% identity to SEQ ID NO: 40. In some embodiments, the immunoglobulin light chain comprises an amino acid sequence with at least 90%, 95%, 96%, 97%, 98%, 99%, or 100% identity to SEQ ID NO: 41 and the immunoglobulin heavy chain comprises an amino acid sequence with at least 90%, 95%, 96%, 97%, 98%, 99%, or 100% identity to SEQ ID NO: 42. In some embodiments, the immunoglobulin light chain comprises an amino acid sequence with at least 90%, 95%, 96%, 97%, 98%, 99%, or 100% identity to SEQ ID NO: 43 and the immunoglobulin heavy chain comprises an amino acid sequence with at least 90%, 95%, 96%, 97%, 98%, 99%, or 100% identity to SEQ ID NO: 44. In some embodiments, the immunoglobulin light chain comprises an amino acid sequence with at least 90%, 95%, 96%, 97%, 98%, 99%, or 100% identity to SEQ ID NO: 45 and the immunoglobulin heavy chain comprises an amino acid sequence with at least 90%, 95%, 96%, 97%, 98%, 99%, or 100% identity to SEQ ID NO: 46. In some embodiments, the immunoglobulin light chain comprises an amino acid sequence with at least 90%, 95%, 96%, 97%, 98%, 99%, or 100% identity to SEQ ID NO: 47 and the immunoglobulin heavy chain comprises an amino acid sequence with at least 90%, 95%, 96%, 97%, 98%, 99%, or 100% identity to SEQ ID NO: 48. In some embodiments, the immunoglobulin light chain comprises an amino acid sequence with at least 90%, 95%, 96%, 97%, 98%, 99%, or 100% identity to SEQ ID NO: 49 and the immunoglobulin heavy chain comprises an amino acid sequence with at least 90%, 95%, 96%, 97%, 98%, 99%, or 100% identity to SEQ ID NO: 50. In some embodiments, the immunoglobulin light chain comprises an amino acid sequence with at least 90%, 95%, 96%, 97%, 98%, 99%, or 100% identity to SEQ ID NO: 51 and the immunoglobulin heavy chain comprises an amino acid sequence with at least 90%, 95%, 96%, 97%, 98%, 99%, or 100% identity to SEQ ID NO: 52. In some embodiments, the immunoglobulin light chain comprises an amino acid sequence with at least 90%, 95%, 96%, 97%, 98%, 99%, or 100% identity to SEQ ID NO: 53 and the immunoglobulin heavy chain comprises an amino acid sequence with at least 90%, 95%, 96%, 97%, 98%, 99%, or 100% identity to SEQ ID NO: 54. In some embodiments, the immunoglobulin light chain comprises an amino acid sequence with at least 90%, 95%, 96%, 97%, 98%, 99%, or 100% identity to SEQ ID NO: 55 and the immunoglobulin heavy chain comprises an amino acid sequence with at least 90%, 95%, 96%, 97%, 98%, 99%, or 100% identity to SEQ ID NO: 56. In some embodiments, the immunoglobulin light chain comprises an amino acid sequence with at least 90%, 95%, 96%, 97%, 98%, 99%, or 100% identity to SEQ ID NO: 57 and the immunoglobulin heavy chain comprises an amino acid sequence with at least 90%, 95%, 96%, 97%, 98%, 99%, or 100% identity to SEQ ID NO: 58. In some embodiments, the immunoglobulin light chain comprises an amino acid sequence with at least 90%, 95%, 96%, 97%, 98%, 99%, or 100% identity to SEQ ID NO: 59 and the immunoglobulin heavy chain comprises an amino acid sequence with at least 90%, 95%, 96%, 97%, 98%, 99%, or 100% identity to SEQ ID NO: 60. In some embodiments, the immunoglobulin light chain comprises an amino acid sequence with at least 90%, 95%, 96%, 97%, 98%, 99%, or 100% identity to SEQ ID NO: 61 and the immunoglobulin heavy chain comprises an amino acid sequence with at least 90%, 95%, 96%, 97%, 98%, 99%, or 100% identity to SEQ ID NO: 62. In some embodiments, the immunoglobulin light chain comprises an amino acid sequence with at least 90%, 95%, 96%, 97%, 98%, 99%, or 100% identity to SEQ ID NO: 63 and the immunoglobulin heavy chain comprises an amino acid sequence with at least 90%, 95%, 96%, 97%, 98%, 99%, or 100% identity to SEQ ID NO: 64. In some embodiments, the immunoglobulin light chain comprises an amino acid sequence with at least 90%, 95%, 96%, 97%, 98%, 99%, or 100% identity to SEQ ID NO: 65 and the immunoglobulin heavy chain comprises an amino acid sequence with at least 90%, 95%, 96%, 97%, 98%, 99%, or 100% identity to SEQ ID NO: 66. In some embodiments, the immunoglobulin light chain comprises an amino acid sequence with at least 90%, 95%, 96%, 97%, 98%, 99%, or 100% identity to SEQ ID NO: 67 and the immunoglobulin heavy chain comprises an amino acid sequence with at least 90%, 95%, 96%, 97%, 98%, 99%, or 100% identity to SEQ ID NO: 68. In some embodiments, the immunoglobulin light chain comprises an amino acid sequence with at least 90%, 95%, 96%, 97%, 98%, 99%, or 100% identity to SEQ ID NO: 69 and the immunoglobulin heavy chain comprises an amino acid sequence with at least 90%, 95%, 96%, 97%, 98%, 99%, or 100% identity to SEQ ID NO: 70. In some embodiments, the immunoglobulin light chain comprises an amino acid sequence with at least 90%, 95%, 96%, 97%, 98%, 99%, or 100% identity to SEQ ID NO: 71 and the immunoglobulin heavy chain comprises an amino acid sequence with at least 90%, 95%, 96%, 97%, 98%, 99%, or 100% identity to SEQ ID NO: 72. In some embodiments, the immunoglobulin light chain comprises an amino acid sequence with at least 90%, 95%, 96%, 97%, 98%, 99%, or 100% identity to SEQ ID NO: 73 and the immunoglobulin heavy chain comprises an amino acid sequence with at least 90%, 95%, 96%, 97%, 98%, 99%, or 100% identity to SEQ ID NO: 74.

In some embodiments, the TROP2 binding domain has weaker binding to TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions. In some embodiments, the TROP2 binding domain has an increased EC₅₀ for TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions. In some embodiments, the TROP2 binding domain has a faster off rate (larger k_{diss}) for TROP2 binding as compared to a TROP2 binding domain that comprises an immunoglobulin light chain according to SEQ ID NO: 29 and an immunoglobulin heavy chain according to SEQ ID NO: 30 as measured under substantially similar kinetic assay conditions.

In some embodiments, the recombinant antibody or antigen binding fragment further comprises a CD3 binding domain. In some embodiments, the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 75; wherein CDR2-L comprises the amino acid sequence of SEQ ID NO: 76; wherein CDR3-L comprises the amino acid sequence of SEQ ID NO: 77; wherein CDR1-H comprises the amino acid sequence of SEQ ID NO: 78; wherein CDR2-H comprises the amino acid sequence of SEQ ID NO: 79; and wherein CDR3-H comprises the amino acid sequence of SEQ ID NO: 80.

In some embodiments, the CD3 binding domain comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H; wherein CDR1-L comprises the amino acid sequence of SEQ ID NO: 75; wherein CDR2-L comprises the amino acid sequence of SEQ ID NO: 76; wherein CDR3-L comprises the amino acid sequence of Z₁-Z₂-W-Z₃-Z₄-Z₅-Z₆-W-Z₇-Z₈; wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 78; wherein CDR2-H comprises the amino acid sequence of SEQ ID NO: 79; wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁; wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A. In some embodiments, Z₁ is V, G, L, I, or A; Z₂ is L, V, I, or A; Z₃ is Y, W, F, or A; Z₄ is S, G, T, or A; Z₅ is N, Q, D, E, or A; Z₆ is R, K, or A; Z₇ is V, G, L, I, or A; Z₈ is F, Y, W, or A; Z₉ is V, G, L, I, or A; Z₁₀ is R, K, or A; Z₁₂ is G, S, T, or A; Z₁₃ is F, Y, W, or A; Z₁₄ is G, S, T, or A; Z₁₅ is N, Q, D, E, or A; Z₁₆ is S, G, T, or A; Z₁₇ is Y, W, F, or A; Z₁₈ is I, V, L, or A; Z₉ is S, G, T, or A; Z₂₀ is W, Y, F, or A; and Z₂₁ is Y, W, F, or A. In some embodiments, Z₈ is F. In some embodiments, Z₁₀ is R; Z₁₁ is H; Z₁₃ is F; Z₁₈ is I; Z₁₉ is S; and Z₂₀ is W.

In some embodiments, CDR3-L comprises an amino acid sequence selected from SEQ ID NOs: 77, 105-106, 108-111, and 113-114. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; and CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; and CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80.

In some embodiments, CDR3-H comprises an amino acid sequence selected from SEQ ID NOs: 80, 116-119, 121-127, and 129-130. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

In some embodiments, the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 107, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 112, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 120; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 128; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

In some embodiments, the immunoglobulin light chain of the CD3 binding domain comprises a variable domain of an immunoglobulin kappa (IgK) or immunoglobulin lambda (IgL) light chain. In some embodiments, the immunoglobulin heavy chain of the CD3 binding domain comprises a variable domain of an IgG1, IgG2, IgG3, or IgG4 heavy chain. In some embodiments, the immunoglobulin light chain of the CD3 binding domain comprises an amino acid sequence with at least 90%, 95%, 96%, 97%, 98%, 99%, or 100% identity to SEQ ID NO: 81. In some embodiments, the immunoglobulin heavy chain of the CD3 binding domain comprises an amino acid sequence with at least 90%, 95%, 96%, 97%, 98%, 99%, or 100% identity to SEQ ID NO: 82. In some embodiments, the immunoglobulin light chain of the CD3 binding domain comprises an amino acid sequence with at least 90%, 95%, 96%, 97%, 98%, 99%, or 100% identity to SEQ ID NO: 81 and the immunoglobulin heavy chain of the CD3 binding domain comprises an amino acid sequence with at least 90%, 95%, 96%, 97%, 98%, 99%, or 100% identity to SEQ ID NO: 82. In some embodiments, the CD3 binding domain comprises a Fab, Fab′, (Fab′)₂ or a single chain variable fragment (scFv). In some embodiments, the CD3 binding domain is the scFv. In some embodiments, the CD3 binding domain comprises an amino acid sequence with at least 90%, 95%, 96%, 97%, 98%, 99%, or 100% identity to SEQ ID NO: 99.

In some embodiments, the CD3 binding domain comprises an amino acid sequence with at least 90%, 95%, 96%, 97%, 98%, 99%, or 100% identity to SEQ ID NO: 131. In some embodiments, the CD3 binding domain comprises an amino acid sequence with at least 90%, 95%, 96%, 97%, 98%, 99%, or 100% identity to SEQ ID NO: 132. In some embodiments, the CD3 binding domain comprises an amino acid sequence with at least 90%, 95%, 96%, 97%, 98%, 99%, or 100% identity to SEQ ID NO: 133. In some embodiments, the CD3 binding domain comprises an amino acid sequence with at least 90%, 95%, 96%, 97%, 98%, 99%, or 100% identity to SEQ ID NO: 134. In some embodiments, the CD3 binding domain comprises an amino acid sequence with at least 90%, 95%, 96%, 97%, 98%, 99%, or 100% identity to SEQ ID NO: 135. In some embodiments, the CD3 binding domain comprises an amino acid sequence with at least 90%, 95%, 96%, 97%, 98%, 99%, or 100% identity to SEQ ID NO: 136. In some embodiments, the CD3 binding domain comprises an amino acid sequence with at least 90%, 95%, 96%, 97%, 98%, 99%, or 100% identity to SEQ ID NO: 137. In some embodiments, the CD3 binding domain comprises an amino acid sequence with at least 90%, 95%, 96%, 97%, 98%, 99%, or 100% identity to SEQ ID NO: 138. In some embodiments, the CD3 binding domain comprises an amino acid sequence with at least 90%, 95%, 96%, 97%, 98%, 99%, or 100% identity to SEQ ID NO: 139. In some embodiments, the CD3 binding domain comprises an amino acid sequence with at least 90%, 95%, 96%, 97%, 98%, 99%, or 100% identity to SEQ ID NO: 140. In some embodiments, the CD3 binding domain comprises an amino acid sequence with at least 90%, 95%, 96%, 97%, 98%, 99%, or 100% identity to SEQ ID NO: 141. In some embodiments, the CD3 binding domain comprises an amino acid sequence with at least 90%, 95%, 96%, 97%, 98%, 99%, or 100% identity to SEQ ID NO: 142. In some embodiments, the CD3 binding domain comprises an amino acid sequence with at least 90%, 95%, 96%, 97%, 98%, 99%, or 100% identity to SEQ ID NO: 143. In some embodiments, the CD3 binding domain comprises an amino acid sequence with at least 90%, 95%, 96%, 97%, 98%, 99%, or 100% identity to SEQ ID NO: 144. In some embodiments, the CD3 binding domain comprises an amino acid sequence with at least 90%, 95%, 96%, 97%, 98%, 99%, or 100% identity to SEQ ID NO: 145. In some embodiments, the CD3 binding domain comprises an amino acid sequence with at least 90%, 95%, 96%, 97%, 98%, 99%, or 100% identity to SEQ ID NO: 146. In some embodiments, the CD3 binding domain comprises an amino acid sequence with at least 90%, 95%, 96%, 97%, 98%, 99%, or 100% identity to SEQ ID NO: 147. In some embodiments, the CD3 binding domain comprises an amino acid sequence with at least 90%, 95%, 96%, 97%, 98%, 99%, or 100% identity to SEQ ID NO: 148. In some embodiments, the CD3 binding domain comprises an amino acid sequence with at least 90%, 95%, 96%, 97%, 98%, 99%, or 100% identity to SEQ ID NO: 149. In some embodiments, the CD3 binding domain comprises an amino acid sequence with at least 90%, 95%, 96%, 97%, 98%, 99%, or 100% identity to SEQ ID NO: 150. In some embodiments, the CD3 binding domain comprises an amino acid sequence with at least 90%, 95%, 96%, 97%, 98%, 99%, or 100% identity to SEQ ID NO: 151. In some embodiments, the CD3 binding domain comprises an amino acid sequence with at least 90%, 95%, 96%, 97%, 98%, 99%, or 100% identity to SEQ ID NO: 152. In some embodiments, the CD3 binding domain comprises an amino acid sequence with at least 90%, 95%, 96%, 97%, 98%, 99%, or 100% identity to SEQ ID NO: 153. In some embodiments, the CD3 binding domain comprises an amino acid sequence with at least 90%, 95%, 96%, 97%, 98%, 99%, or 100% identity to SEQ ID NO: 154. In some embodiments, the CD3 binding domain comprises an amino acid sequence with at least 90%, 95%, 96%, 97%, 98%, 99%, or 100% identity to SEQ ID NO: 155.

In some embodiments, the N-term of the immunoglobulin heavy chain of the TROP2 binding domain is bound to the C-term of the immunoglobulin light chain of the CD3 binding domain. In some embodiments, the C-term of the immunoglobulin heavy chain of the TROP2 binding domain is bound to the N-term of the immunoglobulin light chain of the CD3 binding domain. In some embodiments, the N-term of the immunoglobulin heavy chain of the TROP2 binding domain is bound to the C-term of the immunoglobulin heavy chain of the CD3 binding domain. In some embodiments, the C-term of the immunoglobulin heavy chain of the TROP2 binding domain is bound to the N-term of the immunoglobulin heavy chain of the CD3 binding domain. In some embodiments, the N-term of the immunoglobulin light chain of the TROP2 binding domain is bound to the C-term of the immunoglobulin light chain of the CD3 binding domain. In some embodiments, the C-term of the immunoglobulin light chain of the TROP2 binding domain is bound to the N-term of the immunoglobulin light chain of the CD3 binding domain. In some embodiments, the N-term of the immunoglobulin light chain of the TROP2 binding domain is bound to the C-term of the immunoglobulin heavy chain of the CD3 binding domain. In some embodiments, the C-term of the immunoglobulin light chain of the TROP2 binding domain is bound to the N-term of the immunoglobulin heavy chain of the CD3 binding domain. In some embodiments, the CD3 binding domain is a scFv and the TROP2 binding domain is a Fab or Fab′. In some embodiments, the scFv is bound to the immunoglobulin heavy chain of the Fab or Fab′. In some embodiments, the scFv is bound to the immunoglobulin light chain of the Fab or Fab′. In some embodiments, the immunoglobulin light chain of the scFv is bound to the immunoglobulin heavy chain of the Fab or Fab′. In some embodiments, the immunoglobulin light chain of the scFv is bound to the immunoglobulin light chain of the Fab or Fab′. In some embodiments, the immunoglobulin heavy chain of the scFv is bound to the immunoglobulin heavy chain of the Fab or Fab′. In some embodiments, the immunoglobulin heavy chain of the scFv is bound to the immunoglobulin light chain of the Fab or Fab′.

In some embodiments, the recombinant antibody or antigen binding fragment thereof comprises amino acid sequences with at least 90%, 95%, 96%, 97%, 98%, 99%, or 100% identity to SEQ ID NO: 87 and SEQ ID NO: 88. In some embodiments, the recombinant antibody or antigen binding fragment thereof comprises amino acid sequences with at least 90%, 95%, 96%, 97%, 98%, 99%, or 100% identity to SEQ ID NO: 89 and SEQ ID NO: 90. In some embodiments, the recombinant antibody or antigen binding fragment thereof comprises amino acid sequences with at least 90%, 95%, 96%, 97%, 98%, 99%, or 100% identity to SEQ ID NO: 91 and SEQ ID NO: 92. In some embodiments, the recombinant antibody or antigen binding fragment thereof comprises amino acid sequences with at least 90%, 95%, 96%, 97%, 98%, 99%, or 100% identity to SEQ ID NO: 93 and SEQ ID NO: 94. In some embodiments, the recombinant antibody or antigen binding fragment thereof comprises amino acid sequences with at least 90%, 95%, 96%, 97%, 98%, 99%, or 100% identity to SEQ ID NO: 95 and SEQ ID NO: 96. In some embodiments, the recombinant antibody or antigen binding fragment thereof comprises amino acid sequences with at least 90%, 95%, 96%, 97%, 98%, 99%, or 100% identity to SEQ ID NO: 97 and SEQ ID NO: 98.

In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 188. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 189. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 190. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 194. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 195. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 196.

In some embodiments, the recombinant antibody or antigen binding fragment thereof has weaker cytotoxicity activity as compared to a recombinant antibody or antigen binding fragment thereof that comprises an immunoglobulin light chain according to SEQ ID NO: 83 or 85 and an immunoglobulin heavy chain according to SEQ ID NO: 84 or 86 as measured in an in vitro tumor cell killing assay under substantially similar assay conditions.

Disclosed herein are pharmaceutical compositions comprising: (i) the recombinant antibody or antigen binding fragment thereof of any one of the embodiments; and (ii) a pharmaceutically acceptable excipient.

Disclosed herein are isolated recombinant nucleic acid molecules encoding a polypeptide of the recombinant antibody or antigen binding fragment thereof of any one of the above embodiments.

Disclosed herein are methods of treating a cancer in a subject in need thereof comprising administering to the subject the recombinant antibody or antigen binding fragment thereof of any one of embodiments. In some embodiments, the cancer comprises breast cancer, lung cancer, urothelial cancer, endometrial cancer, ovarian cancer, prostate cancer, pancreatic cancer, gastric cancer, colon cancer, head and neck cancer, or glioma. In some embodiments, the breast cancer comprises triple-negative breast cancer. In some embodiments, the lung cancer comprises non-small cell lung cancer.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1J illustrate biolayer interferometry (BLI) titration data for TROP2 binding by non-mutated and mutated TROP2 binding domains (TBDs).

FIGS. 2A-2M illustrate BLI titration data for TROP2 binding by mutated TBDs.

FIG. 3 illustrates binding curves and EC₅₀s for binding of TROP2 by non-mutated and mutated TBDs as measured by ELISA.

FIG. 4 illustrates binding curves and EC₅₀s for binding of TROP2 by non-mutated and mutated TBDs as measured by ELISA.

FIGS. 5A-5D illustrate BLI titration data for TROP2 binding by non-mutated and mutated T-cell engagers (TCEs).

FIGS. 6A-6D illustrate BLI titration data for TROP2 binding by non-mutated and mutated TCEs.

FIG. 7 illustrates binding curves and EC₅₀s for binding of TROP2 by non-mutated TCEs as measured by ELISA.

FIG. 8 illustrates binding curves and EC₅₀s for binding of CD36 by non-mutated TCEs as measured by ELISA.

FIG. 9 illustrates binding curves and EC₅₀s for binding of TROP2 by mutated TCEs as measured by ELISA.

FIG. 10 illustrates binding curves and EC₅₀s for binding of CD3ε by mutated TCEs as measured by ELISA.

FIG. 11 illustrates binding curves and EC₅₀s for binding of TROP2 by mutated TCEs as measured by ELISA.

FIG. 12 illustrates binding curves and EC₅₀s for binding of CD3ε by mutated TCEs as measured by ELISA.

FIGS. 13A-13F illustrate killing of TROP2 positive HCT116 tumor cells by TCEs in the presence of CD8+ T-cells.

FIGS. 14A-14F illustrate killing of TROP2 positive MDAMB231 tumor cells by TCEs in the presence of CD8+ T-cells.

FIGS. 15A-15E illustrate killing of TROP2 positive NCI-H292 tumor cells by TCEs in the presence of CD8+ T-cells.

FIG. 16 illustrates binding curves and EC₅₀s for binding of human CD3 by TCE-9 to TCE-19 as measured by ELISA.

FIG. 17 illustrates binding curves and EC₅₀s for binding of human CD3 by TCE-9 and TCE-20 to TCE-29 as measured by ELISA.

FIG. 18 illustrates binding curves and EC₅₀s for binding of human CD3 by TCE-9 and TCE-30 to TCE-36 as measured by ELISA.

FIG. 19 illustrates binding curves and EC₅₀s for binding of CD3 binding by TCE-37 to TCE-42 as measured by ELISA.

FIGS. 20A-20B illustrate binding curves and EC₅₀s for binding of human CD3 (FIG. 20A) and cyno CD3 (FIG. 20B) by TCE-37, TCE-39, TCE-40, and TCE-42 as measured by ELISA.

FIG. 21 illustrates binding curves and EC₅₀s for binding of TROP2 by TCE-37, TCE-43, TCE-38, TCE-39, and TCE-40 as measured by ELISA.

FIG. 22 illustrates binding curves and EC₅₀s for binding of CD3 by TCE-37, TCE-43, TCE-38, TCE-39, and TCE-40 as measured by ELISA.

FIG. 23 illustrates binding curves and EC₅₀s for binding of TROP2 by TCE-42, TCE-45, TCE-46, and TCE-44 as measured by ELISA.

FIG. 24 illustrates binding curves and EC₅₀s for binding of CD3 by TCE-42, TCE-45, TCE-46, and TCE-44 as measured by ELISA.

FIGS. 25A-25O illustrate sensorgrams for TCE-10 to TCE-24 binding to CD3ε as measured by biolayer interferometry (BLI).

FIGS. 26A-26N illustrate sensorgrams for TCE-25 to TCE-36 and TCE-9 binding to CD3ε as measured by BLI.

FIGS. 27A-27D illustrate sensorgrams for TCE-11, TCE-12, TCE-15, and TCE-34 binding to CD3ε as measured by BLI.

FIGS. 28A-28B illustrate sensorgrams for TCE-36 and TCE-9 binding to CD3ε as measured by BLI.

FIGS. 29A-29B illustrate sensorgrams for TCE-37 binding to TROP2 (FIG. 29A) and CD3ε (FIG. 29B) and calculated binding affinities (K_{D}), association rates (kₒₙ), and dissociation rates (k_{dis}) as measured by BLI.

FIGS. 30A-30B illustrate sensorgrams for TCE-38 (FIG. 30A) and TCE-39 (FIG. 30B) binding to TROP2 as measured by BLI.

FIGS. 31A-31B illustrate sensorgrams for TCE-38 (FIG. 31A) and TCE-39 (FIG. 31B) binding to CD3ε as measured by BLI.

FIGS. 32A-32B illustrate sensorgrams for TCE-40 binding to TROP2 (FIG. 32A) and CD3ε (FIG. 32B) as measured by BLI.

FIGS. 33A-33B illustrate sensorgrams for TCE-41 and TCE-42 binding to CD36 as measured by BLI.

FIGS. 34A-34B illustrate sensorgrams for TCE-41 and TCE-42 binding to TROP2 as measured by BLI.

FIGS. 35A-35C illustrate sensorgrams for TCE-9, TCE-37, and TCE-43 binding to TROP2 as measured by BLI.

FIG. 36 illustrates killing of TROP2 positive NCI-H292 tumor cells by TCEs in the presence of CD8+ T-cells.

FIG. 37 illustrates killing of TROP2 positive NCI-H292 tumor cells by TCEs in the presence of CD8+ T-cells.

FIG. 38 illustrates killing of TROP2 positive NCI-H292 tumor cells by TCEs in the presence of CD8+ T-cells.

FIG. 39 illustrates killing of TROP2 positive HCT116 tumor cells by TCEs in the presence of CD8+ T-cells.

FIG. 40 illustrates killing of TROP2 positive NCI-H292 tumor cells by TCEs in the presence of CD8+ T-cells.

FIG. 41 illustrates killing of cynomolgus TROP2 positive HEK293 cells by TCEs in the presence of CD8+ T-cells.

FIG. 42 illustrates killing of TROP2 positive NCI-H292 tumor cells by TCEs in the presence of CD8+ T-cells.

FIG. 43 illustrates killing of TROP2 positive NCI-H292 tumor cells by a TCE in the presence of CD8+ T-cells.

FIG. 44 illustrates killing of cynomolgus TROP2 positive HEK293 cells by TCEs in the presence of CD8+ T-cells.

FIG. 45 illustrates killing of TROP2 positive NCI-H292 tumor cells by TCEs in the presence of CD8+ T-cells.

FIG. 46 illustrates killing of cynomolgus TROP2 positive HEK293 cells by TCEs in the presence of CD8+ T-cells.

FIG. 47 illustrates killing of TROP2 positive NCI-H292 tumor cells by TCEs in the presence of CD8+ T-cells.

FIG. 48 illustrates killing of cynomolgus TROP2 positive HEK293 cells by TCEs in the presence of CD8+ T-cells.

FIG. 49 illustrates results of pharmacokinetic and toxicity studies of TCE-43 in cynomolgus monkey.

FIG. 50 illustrates results of pharmacokinetic and toxicity studies of TCE-37 in cynomolgus monkey.

FIG. 51 shows cytokine release in cynomolgus monkey after continuous IV infusion of TCE-43.

FIG. 52 shows cytokine release in cynomolgus monkey after continuous IV infusion of TCE-37.

FIGS. 53A-53F show clinical chemistry parameters in cynomolgus monkeys after continuous IV infusion of TCE-43.

FIGS. 54A-54F show clinical chemistry parameters in cynomolgus monkeys after continuous IV infusion of TCE-37.

FIG. 55 illustrates results of pharmacokinetic and toxicity studies of TCE-39 in cynomolgus monkey.

FIG. 56 illustrates results of pharmacokinetic and toxicity studies of TCE-42 in cynomolgus monkey.

FIG. 57 illustrates results of pharmacokinetic and toxicity studies of TCE-40 in cynomolgus monkey.

FIGS. 58A-58D show cytokine release in cynomolgus monkeys after continuous IV infusion of TCE-39.

FIGS. 59A-59D show cytokine release in cynomolgus monkeys after continuous IV infusion of TCE-42.

FIG. 60 shows cytokine release in cynomolgus monkeys after continuous IV infusion of TCE-40.

FIGS. 61A-61F show clinical chemistry parameters in cynomolgus monkeys after continuous IV infusion of TCE-39.

FIGS. 62A-62F show clinical chemistry parameters in cynomolgus monkeys after continuous IV infusion of TCE-42.

FIGS. 63A-63F show clinical chemistry parameters in cynomolgus monkeys after continuous IV infusion of TCE-40.

### DETAILED DESCRIPTION OF THE INVENTION

TROP2 (also known as tumor-associated calcium signal transducer 2 or epithelial glycoprotein-1) is the protein product of the TACSTD2 gene, and is a transmembrane glycoprotein that functions in variety of cell signaling pathways, many of which are associated with tumorigenesis. TROP2 has been shown to be overexpressed in multiple human carcinomas, including lung, breast, cervical, ovarian, colorectal, pancreatic, and gastric cancers, and its expression has been correlated with poor patient prognosis. Furthermore, TROP2 functions as an oncogene capable of driving both tumorigenesis and metastasis in epithelial cancers such as colorectal cancer. TROP2 expression in cancer cells has long been correlated with drug resistance, and high levels of TROP2 expression have been shown to correlate with poor prognosis in a variety of cancer types. In a meta-analysis, including data from approximately 2,500 patients, increased TROP2 expression was associated with poor overall survival and disease-free survival outcomes across several solid tumors.

Disclosed herein are recombinant antibodies or antigen binding fragments thereof that comprise a tumor-associated calcium signal transducer 2 (TROP2) binding domain that has been optimized for certain properties. In some embodiments, the TROP2 binding domain has TROP2 binding properties that improve safety and reduce accumulation in healthy tissue. For instance, in some embodiments, the TROP2 binding domain has faster off rates (k_{diss}) and/or weaker (higher) EC₅₀s for TROP2 binding which may help reduce accumulation in healthy tissues. Furthermore, in some embodiments, the TROP2 binding domain has faster off rates (k_{diss}) and/or weaker (higher) EC₅₀s for TROP2 binding while maintaining TROP2 binding capabilities and activity. In some embodiments, the recombinant antibodies or antigen binding fragments thereof exhibit weaker cytotoxicity activity to help reduce off tumor on target toxicity in healthy tissues.

Disclosed herein are recombinant antibodies or antigen binding fragments thereof that comprise a tumor-associated calcium signal transducer 2 (TROP2) binding domain that have been optimized through alanine scanning mutagenesis. Alanine scanning of the TROP2 binding domain was performed to optimize certain properties of the recombinant antibodies or antigen binding fragments thereof. Alanine scanning was accomplished by systematically mutating individual residues to alanine in the TROP2 binding domain relative to a non-mutated or “wild-type” TROP2 binding domain. Specifically, residues in the third complementarity determining regions (CDR3) of the immunoglobulin light chain and the immunoglobulin heavy chain were systematically mutated to alanine in order to both establish CDR3 related structure activity relationships (SAR) and to identify TROP2 binding domains that maintain binding and activity while exhibiting faster off rates to reduce accumulation in healthy tissues.

In some embodiments, the recombinant antibodies or antigen fragments that comprise a TROP2 binding domain that have been optimized for certain properties further comprise a CD3 binding domain. Multispecific antibodies for redirecting T cells to cancers have shown promise in both pre-clinical and clinical studies. This approach relies on binding of one antigen interacting portion of the antibody to a tumor-associated antigen or marker such as TROP2, while a second antigen interacting portion can bind to an effector cell antigen on a T cell, such as CD3, which then triggers cytotoxic activity. Disclosed herein are multispecific antibody compositions that target TROP2 and CD3 for triggering cytotoxic activity.

In some embodiments, the recombinant antibodies or antigen binding fragments thereof described herein are used in a method of treating cancer. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric. In some embodiments, the polypeptides or polypeptide complexes described herein are used in a method of treating triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer. In some embodiments, the polypeptides or polypeptide complexes described herein are used in a method of treating breast cancer, lung cancer, urothelial cancer, endometrial cancer, ovarian cancer, prostate cancer, pancreatic cancer, gastric cancer, colon cancer, head and neck cancer, and glioma.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### Recombinant Antibodies or Antigen Binding Fragment Compositions

### TROP2 Binding Domain

Disclosed herein are recombinant antibodies or antigen binding fragments thereof that comprise a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2, and wherein CDR3-L comprises an amino acid sequence of X₁X₂HYX₃X₄X₅X₆X₇ wherein X₁ is Q, S, T, D, N, E, H, R, K, or A, X₂ is Q, S, T, D, N, E, H, K, R, or A, X₃ is I, G, P, V, L, M, S, T, or A, X₄ is T, G, S, M, H, N, Q, or A, X₅ is P, G, V, L, I, M, S, T, or A, X₆ is L, G, P, V, I, M, S, T, or A; and X₇ is T, G, S, M, H, N, Q, or A, wherein CDR1-H comprises the amino acid sequence of SEQ ID NO: 13, wherein CDR2-H comprises the amino acid sequence of SEQ ID NO: 14, and wherein CDR3-H comprises an amino acid sequence of AX₈X₉GX₁₀X₁₁X₁₂X₁₃YWX₁₄X₁₅ X₁₆X₁₇ wherein X₈ is R, S, T, Q, D, E, N, H, K, or A, X₉ is G, P, V, L, I M, S, T, or A, X₁₀ is F, Y, W, V, L, I, G, or A, X₁₁ is G, P, V, L, I, M, S, T, or A, X₁₂ is S, G, T, M, N, Q, H, or A, X₁₃ is S, G, T, M, N, Q, H, or A, X₁₄ is Y, F, W, V, L, I, G, or A, X₁₅ is F, Y, W, V, L, I, G, or A, X₁₆ is D, Q, N, E, S, T, H, K, R, or A, and X₁₇ is V, G, P, L, I, M, S, T, or A, and wherein when CDR3-H comprises the amino acid sequence of SEQ ID NO: 15, then CDR3-L does not comprise the amino acid sequence of SEQ ID NO: 3.

In some embodiments, X₁ is Q, N, D, E, or A, X₂ is Q, N, D, E, or A, X₃ is I, V, L, or A, X₄ is T, S, or A, X₅ is P, G, or A, X₆ is L, V, I, or A, X₇ is T, S, or A, X₈ is R, K, or A, X₉ is G, S, T, or A, X₁₀ is F, Y, or A, X is G, S, T, or A, X₁₂ is S, G, T, or A, X₁₃ is S, G, T, or A, X₁₄ is Y, W, F, or A, X₁₅ is F, Y, W, or A, X₁₆ is D, E, Q, N, or A, and X₁₇ is V, L, I, or A. In some embodiments, X₁ is Q, and X₆ is L. In some embodiments, X₈ is R, X₁₀ is F, X₁₁ is G, X₁₄ is Y, X₁₅ is F, and X₁₆ is D.

In some embodiments, X₁ is Q, S, T, D, N, E, or A; X₂ is Q, S, T, D, N, E, or A; X₃ is I, G, P, V, L, M, or A; X₄ is T, G, S, M, H, N, Q, or A; X₅ is P, G, V, L, I, M, or A; X₆ is L, G, P, V, I, M, or A; X₇ is T, G, S, M, H, N, Q, or A; X₈ is R, H, K, or A; X₉ is G, P, V, L, I, M, S, T, or A; X₁₀ is F, Y, W, V, L, I, or A; X₁ is G, P, V, L, I, M, S, T, or A; X₁₂ is S, G, T, M, N, Q, or A; X₁₃ is S, G, T, M, N, Q, or A; X₁₄ is Y, F, W, V, L, I, or A; X₅ is F, Y, W, V, L, I, or A; X₁₁ is D, Q, N, E, S, T, or A; and X₁₇ is V, G, P, L, I, M, or A. In some embodiments, X₁ is Q, N, or A; X₂ is Q, N, or A; X₃ is I, V, L, or A; X₄ is T, S, or A; X₅ is P, G, or A; X₆ is L, V, I, or A; X₇ is T, S, or A; X₈ is R, K, or A; X₉ is G, V, S, T, or A; X₁₀ is F, Y, or A; X₁₁ is G, V, S, T, or A; X₁₂ is S, G, T, or A; X₁₃ is S, G, T, or A; X₁₄ is Y, W, or A; X₁₅ is F, Y, or A; X₁₆ is D, E, or A; and X₁₇ is V, G, L, I, or A. In some embodiments, X₁ is Q A and X₆ is L. In some embodiments, X₈ is R; X₁₀ is F; X₁₁ is G; X₁ is Y; X₁₅ is F; and X₁₆ is D.

In some embodiments, CDR3-L comprises an amino acid selected from SEQ ID NOs: 3-5 and 8-12. In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2; CDR3-L: SEQ ID NO: 8, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11, and CDR1-H: SEQ ID NO: 13, CDR2-H: SEQ ID NO: 14, CDR3-H: SEQ ID NO: 15; and CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15.

In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15.

In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 8, and CDR1-H: SEQ ID NO: 13, CDR2-H: SEQ ID NO: 14, CDR3-H: SEQ ID NO: 15. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 11, and CDR1-H: SEQ ID NO: 13, CDR2-H: SEQ ID NO: 14, CDR3-H: SEQ ID NO: 15. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15.

In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 5 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; and CDR1-L: SEQ ID NO: 1, CDR2-L: SEQ ID NO: 2, CDR3-L: SEQ ID NO: 12 and CDR1-H: SEQ ID NO: 13, CDR2-H: SEQ ID NO: 14, CDR3-H: SEQ ID NO: 15.

In some embodiments, CDR3-H comprises an amino acid selected from SEQ ID NOs: 16-17, 19-22, 25-28. In some embodiments, the immunoglobulin light chain and the immunoglobulin heavy chain comprise a set of CDRs selected from the group consisting of: CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO:21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 21. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27. In some embodiments, the immunoglobulin light chain and the immunoglobulin heavy chain comprise a set of CDR sequences comprising CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO:21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

Disclosed herein are recombinant antibodies or antigen binding fragments thereof that comprise a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 6 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 7 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 12 and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 18; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO:21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 23; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 24; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

TABLE 1
TROP2 binding domain light chain complementarity determining regions
(CDRs) as based on IMGT CDR numbering system.

| Construct Description | Amino Acid Sequence (N to C) | SEQ ID NO: |
|---|---|---|
| CDR1-L | QDVSIA | 1 |
| CDR2-L | SA | 2 |
| CDR3-L | QQHYITPLT | 3 |
| CDR3-L Q89A | AQHYITPLT | 4 |
| CDR3-L Q90A | QAHYITPLT | 5 |
| CDR3-L H91A | QQAYITPLT | 6 |
| CDR3-L Y92A | QQHAITPLT | 7 |
| CDR3-L I93A | QQHYATPLT | 8 |
| CDR3-L T94A | QQHYIAPLT | 9 |
| CDR3-L P95A | QQHYITALT | 10 |
| CDR3-L L96A | QQHYITPAT | 11 |
| CDR3-L T97A | QQHYITPLA | 12 |

TABLE 2
TROP2 binding domain heavy chain complementarity determining regions
(CDRs) as based on IMGT CDR numbering system.

| Construct Description | Amino Acid Sequence (N to C) | SEQ ID NO: |
|---|---|---|
| CDR1-H | GYTFTNYG | 13 |
| CDR2-H | INTYTGEP | 14 |
| CDR3-H | ARGGFGSSYWYFDV | 15 |
| CDR3-H R98A | AAGGFGSSYWYFDV | 16 |
| CDR3-H G99A | ARAGFGSSYWYFDV | 17 |
| CDR3-H G100A | ARGAFGSSYWYFDV | 18 |
| CDR3-H F101A | ARGGAGSSYWYFDV | 19 |
| CDR3-H G102A | ARGGFASSYWYFDV | 20 |
| CDR3-H S103A | ARGGFGASYWYFDV | 21 |
| CDR3-H S104A | ARGGFGSAYWYFDV | 22 |
| CDR3-H Y105A | ARGGFGSSAWYFDV | 23 |
| CDR3-H W106A | ARGGFGSSYAYFDV | 24 |
| CDR3-H Y107A | ARGGFGSSYWAFDV | 25 |
| CDR3-H F108A | ARGGFGSSYWYADV | 26 |
| CDR3-H D109A | ARGGFGSSYWYFAV | 27 |
| CDR3-H V110A | ARGGFGSSYWYFDA | 28 |

In some embodiments, the TROP2 binding domain comprises a Fab, Fab′, (Fab′)₂ or a single chain variable fragment (scFv). In some embodiments, the TROP2 binding domain is a Fab. In some embodiments, the immunoglobulin light chain comprises a variable domain of immunoglobulin kappa (IgK) or immunoglobulin lambda (IgL) light chain. In some embodiments, the immunoglobulin heavy chain comprises a variable domain of an IgG1, IgG2, IgG3, or IgG4 heavy chain.

In some embodiments, the immunoglobulin light chain comprises an amino acid sequence with at least 90%, 95%, 96%, 97%, 98%, 99%, or 100% identity to any one of SEQ ID NOs: 31, 33, 35, 37, 39, 41, 43, 45, 47, 49, 51, 53, 55, 57, 59, 61, 63, 65, 67, 69, 71, or 73. In some embodiments, the immunoglobulin heavy chain comprises an amino acid sequence with at least 90%, 95%, 96%, 97%, 98%, 99%, or 100% identity to any one of SEQ ID NOs: 32, 34, 36, 38, 40, 42, 44, 46, 48, 50, 52, 54, 56, 58, 60, 62, 64, 66, 68, 70, 72, or 74.

In some embodiments, the immunoglobulin light chain comprises an amino acid sequence with at least 90%, 95%, 96%, 97%, 98%, 99%, or 100% identity to SEQ ID NO: 31 and the immunoglobulin heavy chain comprises an amino acid sequence with at least 90%, 95%, 96%, 97%, 98%, 99%, or 100% identity to SEQ ID NO: 32. In some embodiments, the immunoglobulin light chain comprises an amino acid sequence with at least 90%, 95%, 96%, 97%, 98%, 99%, or 100% identity to SEQ ID NO: 33 and the immunoglobulin heavy chain comprises an amino acid sequence with at least 90%, 95%, 96%, 97%, 98%, 99%, or 100% identity to SEQ ID NO: 34. In some embodiments, the immunoglobulin light chain comprises an amino acid sequence with at least 90%, 95%, 96%, 97%, 98%, 99%, or 100% identity to SEQ ID NO: 35 and the immunoglobulin heavy chain comprises an amino acid sequence with at least 90%, 95%, 96%, 97%, 98%, 99%, or 100% identity to SEQ ID NO: 36. In some embodiments, the immunoglobulin light chain comprises an amino acid sequence with at least 90%, 95%, 96%, 97%, 98%, 99%, or 100% identity to SEQ ID NO: 37 and the immunoglobulin heavy chain comprises an amino acid sequence with at least 90%, 95%, 96%, 97%, 98%, 99%, or 100% identity to SEQ ID NO: 38. In some embodiments, the immunoglobulin light chain comprises an amino acid sequence with at least 90%, 95%, 96%, 97%, 98%, 99%, or 100% identity to SEQ ID NO: 39 and the immunoglobulin heavy chain comprises an amino acid sequence with at least 90%, 95%, 96%, 97%, 98%, 99%, or 100% identity to SEQ ID NO: 40. In some embodiments, the immunoglobulin light chain comprises an amino acid sequence with at least 90%, 95%, 96%, 97%, 98%, 99%, or 100% identity to SEQ ID NO: 41 and the immunoglobulin heavy chain comprises an amino acid sequence with at least 90%, 95%, 96%, 97%, 98%, 99%, or 100% identity to SEQ ID NO: 42. In some embodiments, the immunoglobulin light chain comprises an amino acid sequence with at least 90%, 95%, 96%, 97%, 98%, 99%, or 100% identity to SEQ ID NO: 43 and the immunoglobulin heavy chain comprises an amino acid sequence with at least 90%, 95%, 96%, 97%, 98%, 99%, or 100% identity to SEQ ID NO: 44. In some embodiments, the immunoglobulin light chain comprises an amino acid sequence with at least 90%, 95%, 96%, 97%, 98%, 99%, or 100% identity to SEQ ID NO: 45 and the immunoglobulin heavy chain comprises an amino acid sequence with at least 90%, 95%, 96%, 97%, 98%, 99%, or 100% identity to SEQ ID NO: 46. In some embodiments, the immunoglobulin light chain comprises an amino acid sequence with at least 90%, 95%, 96%, 97%, 98%, 99%, or 100% identity to SEQ ID NO: 47 and the immunoglobulin heavy chain comprises an amino acid sequence with at least 90%, 95%, 96%, 97%, 98%, 99%, or 100% identity to SEQ ID NO: 48.

In some embodiments, the immunoglobulin light chain comprises an amino acid sequence with at least 90%, 95%, 96%, 97%, 98%, 99%, or 100% identity to SEQ ID NO: 49 and the immunoglobulin heavy chain comprises an amino acid sequence with at least 90%, 95%, 96%, 97%, 98%, 99%, or 100% identity to SEQ ID NO: 50. In some embodiments, the immunoglobulin light chain comprises an amino acid sequence with at least 90%, 95%, 96%, 97%, 98%, 99%, or 100% identity to SEQ ID NO: 51 and the immunoglobulin heavy chain comprises an amino acid sequence with at least 90%, 95%, 96%, 97%, 98%, 99%, or 100% identity to SEQ ID NO: 52. In some embodiments, the immunoglobulin light chain comprises an amino acid sequence with at least 90%, 95%, 96%, 97%, 98%, 99%, or 100% identity to SEQ ID NO: 53 and the immunoglobulin heavy chain comprises an amino acid sequence with at least 90%, 95%, 96%, 97%, 98%, 99%, or 100% identity to SEQ ID NO: 54. In some embodiments, the immunoglobulin light chain comprises an amino acid sequence with at least 90%, 95%, 96%, 97%, 98%, 99%, or 100% identity to SEQ ID NO: 55 and the immunoglobulin heavy chain comprises an amino acid sequence with at least 90%, 95%, 96%, 97%, 98%, 99%, or 100% identity to SEQ ID NO: 56. In some embodiments, the immunoglobulin light chain comprises an amino acid sequence with at least 90%, 95%, 96%, 97%, 98%, 99%, or 100% identity to SEQ ID NO: 57 and the immunoglobulin heavy chain comprises an amino acid sequence with at least 90%, 95%, 96%, 97%, 98%, 99%, or 100% identity to SEQ ID NO: 58. In some embodiments, the immunoglobulin light chain comprises an amino acid sequence with at least 90%, 95%, 96%, 97%, 98%, 99%, or 100% identity to SEQ ID NO: 59 and the immunoglobulin heavy chain comprises an amino acid sequence with at least 90%, 95%, 96%, 97%, 98%, 99%, or 100% identity to SEQ ID NO: 60. In some embodiments, the immunoglobulin light chain comprises an amino acid sequence with at least 90%, 95%, 96%, 97%, 98%, 99%, or 100% identity to SEQ ID NO: 61 and the immunoglobulin heavy chain comprises an amino acid sequence with at least 90%, 95%, 96%, 97%, 98%, 99%, or 100% identity to SEQ ID NO: 62. In some embodiments, the immunoglobulin light chain comprises an amino acid sequence with at least 90%, 95%, 96%, 97%, 98%, 99%, or 100% identity to SEQ ID NO: 63 and the immunoglobulin heavy chain comprises an amino acid sequence with at least 90%, 95%, 96%, 97%, 98%, 99%, or 100% identity to SEQ ID NO: 64. In some embodiments, the immunoglobulin light chain comprises an amino acid sequence with at least 90%, 95%, 96%, 97%, 98%, 99%, or 100% identity to SEQ ID NO: 65 and the immunoglobulin heavy chain comprises an amino acid sequence with at least 90%, 95%, 96%, 97%, 98%, 99%, or 100% identity to SEQ ID NO: 66. In some embodiments, the immunoglobulin light chain comprises an amino acid sequence with at least 90%, 95%, 96%, 97%, 98%, 99%, or 100% identity to SEQ ID NO: 67 and the immunoglobulin heavy chain comprises an amino acid sequence with at least 90%, 95%, 96%, 97%, 98%, 99%, or 100% identity to SEQ ID NO: 68. In some embodiments, the immunoglobulin light chain comprises an amino acid sequence with at least 90%, 95%, 96%, 97%, 98%, 99%, or 100% identity to SEQ ID NO: 69 and the immunoglobulin heavy chain comprises an amino acid sequence with at least 90%, 95%, 96%, 97%, 98%, 99%, or 100% identity to SEQ ID NO: 70. In some embodiments, the immunoglobulin light chain comprises an amino acid sequence with at least 90%, 95%, 96%, 97%, 98%, 99%, or 100% identity to SEQ ID NO: 71 and the immunoglobulin heavy chain comprises an amino acid sequence with at least 90%, 95%, 96%, 97%, 98%, 99%, or 100% identity to SEQ ID NO: 72. In some embodiments, the immunoglobulin light chain comprises an amino acid sequence with at least 90%, 95%, 96%, 97%, 98%, 99%, or 10000 identity to SEQ ID NO: 73 and the immunoglobulin heavy chain comprises an amino acid sequence with at least 90%, 95%, 96%, 97%, 98%, 99%, or 100% identity to SEQ ID NO: 74.

TABLE 3
TROP2 binding domain (TBD) heavy chain and light chain sequences

| | | SEQ ID |
|---|---|---|
| Construct Description | Amino Acid Sequence (N to C) | NO: |
| TBD-1 LC | DIQLTQSPSSLSASVGDRVSITCKASQDVSIAVAWYQQKPGK | 29 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-1 HC | QVQLQQSGSELKKPGASVKVSCKASGYTFTNYGMNWVKQ | 30 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-2 LC | DIQLTQSPSSLSASVGDRVSITCKASQDVSIAVAWYQQKPGK | 31 |
| Q89A | APKLLIYSASYRYTGVPDRFSGSGSGTDFTLTISSLQPEDFAV | |
| | YYCAQHYITPLTFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-2 HC | QVQLQQSGSELKKPGASVKVSCKASGYTFTNYGMNWVKQ | 32 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-3 LC | DIQLTQSPSSLSASVGDRVSITCKASQDVSIAVAWYQQKPGK | 33 |
| Q90A | APKLLIYSASYRYTGVPDRFSGSGSGTDFTLTISSLQPEDFAV | |
| | YYCQAHYITPLTFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-3 HC | QVQLQQSGSELKKPGASVKVSCKASGYTFTNYGMNWVKQ | 34 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-4 LC | DIQLTQSPSSLSASVGDRVSITCKASQDVSIAVAWYQQKPGK | 35 |
| H91A | APKLLIYSASYRYTGVPDRFSGSGSGTDFTLTISSLQPEDFAV | |
| | YYCQQAYITPLTFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-4 HC | QVQLQQSGSELKKPGASVKVSCKASGYTFTNYGMNWVKQ | 36 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-5 LC | DIQLTQSPSSLSASVGDRVSITCKASQDVSIAVAWYQQKPGK | 37 |
| Y92A | APKLLIYSASYRYTGVPDRFSGSGSGTDFTLTISSLQPEDFAV | |
| | YYCQQHAITPLTFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-5 HC | QVQLQQSGSELKKPGASVKVSCKASGYTFTNYGMNWVKQ | 38 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-6 LC | DIQLTQSPSSLSASVGDRVSITCKASQDVSIAVAWYQQKPGK | 39 |
| I93A | APKLLIYSASYRYTGVPDRFSGSGSGTDFTLTISSLQPEDFAV | |
| | YYCQQHYATPLTFGAGTKVEIKRTVAAPSVFIFPPSDEQLK | |
| | SGTASVVCLLNNFYPREAKVQWKVDNALQSGNSQESVTEQ | |
| | DSKDSTYSLSSTLTLSKADYEKHKVYACEVTHQGLSSPVTK | |
| | SFNRGEC | |
| TBD-6 HC | QVQLQQSGSELKKPGASVKVSCKASGYTFTNYGMNWVKQ | 40 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-7 LC | DIQLTQSPSSLSASVGDRVSITCKASQDVSIAVAWYQQKPGK | 41 |
| T94A | APKLLIYSASYRYTGVPDRFSGSGSGTDFTLTISSLQPEDFAV | |
| | YYCQQHYIAPLTFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-7 HC | QVQLQQSGSELKKPGASVKVSCKASGYTFTNYGMNWVKQ | 42 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-8 LC | DIQLTQSPSSLSASVGDRVSITCKASQDVSIAVAWYQQKPGK | 43 |
| P95A | APKLLIYSASYRYTGVPDRFSGSGSGTDFTLTISSLQPEDFAV | |
| | YYCQQHYITALTFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-8 HC | QVQLQQSGSELKKPGASVKVSCKASGYTFTNYGMNWVKQ | 44 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-9 LC | DIQLTQSPSSLSASVGDRVSITCKASQDVSIAVAWYQQKPGK | 45 |
| L96A | APKLLIYSASYRYTGVPDRFSGSGSGTDFTLTISSLQPEDFAV | |
| | YYCQQHYITPATFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-9 HC | QVQLQQSGSELKKPGASVKVSCKASGYTFTNYGMNWVKQ | 46 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-10 LC | DIQLTQSPSSLSASVGDRVSITCKASQDVSIAVAWYQQKPGK | 47 |
| T97A | APKLLIYSASYRYTGVPDRFSGSGSGTDFTLTISSLQPEDFAV | |
| | YYCQQHYITPLAFGAGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TBD-10 HC | QVQLQQSGSELKKPGASVKVSCKASGYTFTNYGMNWVKQ | 48 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-11 LC | DIQLTQSPSSLSASVGDRVSITCKASQDVSIAVAWYQQKPGK | 49 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-11 HC | QVQLQQSGSELKKPGASVKVSCKASGYTFTNYGMNWVKQ | 50 |
| R98A | APGQGLKWMGWINTYTGEPTYTDDFKGRFAFSLDTSVSTA | |
| | YLQISSLKADDTAVYFCAAGGFGSSYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-12 LC | DIQLTQSPSSLSASVGDRVSITCKASQDVSIAVAWYQQKPGK | 51 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-12 HC | QVQLQQSGSELKKPGASVKVSCKASGYTFTNYGMNWVKQ | 52 |
| G99A | APGQGLKWMGWINTYTGEPTYTDDFKGRFAFSLDTSVSTA | |
| | YLQISSLKADDTAVYFCARAGFGSSYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-13 LC | DIQLTQSPSSLSASVGDRVSITCKASQDVSIAVAWYQQKPGK | 53 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-13 HC | QVQLQQSGSELKKPGASVKVSCKASGYTFTNYGMNWVKQ | 54 |
| G100A | APGQGLKWMGWINTYTGEPTYTDDFKGRFAFSLDTSVSTA | |
| | YLQISSLKADDTAVYFCARGAFGSSYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-14 LC | DIQLTQSPSSLSASVGDRVSITCKASQDVSIAVAWYQQKPGK | 55 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-14 HC | QVQLQQSGSELKKPGASVKVSCKASGYTFTNYGMNWVKQ | 56 |
| F101A | APGQGLKWMGWINTYTGEPTYTDDFKGRFAFSLDTSVSTA | |
| | YLQISSLKADDTAVYFCARGGAGSSYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-15 LC | DIQLTQSPSSLSASVGDRVSITCKASQDVSIAVAWYQQKPGK | 57 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-15 HC | QVQLQQSGSELKKPGASVKVSCKASGYTFTNYGMNWVKQ | 58 |
| G102A | APGQGLKWMGWINTYTGEPTYTDDFKGRFAFSLDTSVSTA | |
| | YLQISSLKADDTAVYFCARGGFASSYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-16 LC | DIQLTQSPSSLSASVGDRVSITCKASQDVSIAVAWYQQKPGK | 59 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-16 HC | QVQLQQSGSELKKPGASVKVSCKASGYTFTNYGMNWVKQ | 60 |
| S103A | APGQGLKWMGWINTYTGEPTYTDDFKGRFAFSLDTSVSTA | |
| | YLQISSLKADDTAVYFCARGGFGASYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-17 LC | DIQLTQSPSSLSASVGDRVSITCKASQDVSIAVAWYQQKPGK | 61 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-17 HC | QVQLQQSGSELKKPGASVKVSCKASGYTFTNYGMNWVKQ | 62 |
| S104A | APGQGLKWMGWINTYTGEPTYTDDFKGRFAFSLDTSVSTA | |
| | YLQISSLKADDTAVYFCARGGFGSAYWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-18 LC | DIQLTQSPSSLSASVGDRVSITCKASQDVSIAVAWYQQKPGK | 63 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-18 HC | QVQLQQSGSELKKPGASVKVSCKASGYTFTNYGMNWVKQ | 64 |
| Y105A | APGQGLKWMGWINTYTGEPTYTDDFKGRFAFSLDTSVSTA | |
| | YLQISSLKADDTAVYFCARGGFGSSAWYFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-19 LC | DIQLTQSPSSLSASVGDRVSITCKASQDVSIAVAWYQQKPGK | 65 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-19 HC | QVQLQQSGSELKKPGASVKVSCKASGYTFTNYGMNWVKQ | 66 |
| W106A | APGQGLKWMGWINTYTGEPTYTDDFKGRFAFSLDTSVSTA | |
| | YLQISSLKADDTAVYFCARGGFGSSYAYFDVWGQGSLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTYI | |
| | CNVNHKPSNTKVDKKVEPKSC | |
| TBD-20 LC | DIQLTQSPSSLSASVGDRVSITCKASQDVSIAVAWYQQKPGK | 67 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-20 HC | QVQLQQSGSELKKPGASVKVSCKASGYTFTNYGMNWVKQ | 68 |
| Y107A | APGQGLKWMGWINTYTGEPTYTDDFKGRFAFSLDTSVSTA | |
| | YLQISSLKADDTAVYFCARGGFGSSYWAFDVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-21 LC | DIQLTQSPSSLSASVGDRVSITCKASQDVSIAVAWYQQKPGK | 69 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-21 HC | QVQLQQSGSELKKPGASVKVSCKASGYTFTNYGMNWVKQ | 70 |
| F108A | APGQGLKWMGWINTYTGEPTYTDDFKGRFAFSLDTSVSTA | |
| | YLQISSLKADDTAVYFCARGGFGSSYWYADVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-22 LC | DIQLTQSPSSLSASVGDRVSITCKASQDVSIAVAWYQQKPGK | 71 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-22 HC | QVQLQQSGSELKKPGASVKVSCKASGYTFTNYGMNWVKQ | 72 |
| D109A | APGQGLKWMGWINTYTGEPTYTDDFKGRFAFSLDTSVSTA | |
| | YLQISSLKADDTAVYFCARGGFGSSYWYFAVWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |
| TBD-23 LC | DIQLTQSPSSLSASVGDRVSITCKASQDVSIAVAWYQQKPGK | 73 |
| | APKLLIYSASYRYTGVPDRFSGSGSGTDFTLTISSLQPEDFAV | |
| | YYCQQHYITPLTFGAGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-23 HC | QVQLQQSGSELKKPGASVKVSCKASGYTFTNYGMNWVKQ | 74 |
| V110A | APGQGLKWMGWINTYTGEPTYTDDFKGRFAFSLDTSVSTA | |
| | YLQISSLKADDTAVYFCARGGFGSSYWYFDAWGQGSLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSC | |

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 4, and

CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 31 and the immunoglobulin heavy chain comprises an amino acid sequence with at least 90% identity to SEQ ID NO: 32.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 5 and CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 33 and the immunoglobulin heavy chain comprises an amino acid sequence with at least 90% identity to SEQ ID NO: 34.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 8 and CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 39 and the immunoglobulin heavy chain comprises an amino acid sequence with at least 90% identity to SEQ ID NO: 40.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 9 and CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 41 and the immunoglobulin heavy chain comprises an amino acid sequence with at least 90% identity to SEQ ID NO: 42.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 10 and

CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 43 and the immunoglobulin heavy chain comprises an amino acid sequence with at least 90% identity to SEQ ID NO: 44.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 11 and CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 45 and the immunoglobulin heavy chain comprises an amino acid sequence with at least 90% identity to SEQ ID NO: 46.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 12 and CDR1-H: SEQ ID NO: 13, CDR2-H: SEQ ID NO: 14, CDR3-H: SEQ ID NO: 15; and the immunoglobulin light chain comprises an amino acid sequence with at least 90% identity to SEQ ID NO: 47 and the immunoglobulin heavy chain comprises an amino acid sequence with at least 90% identity to SEQ ID NO: 48.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; and the immunoglobulin light chain comprises an amino acid sequence with at least 90% identity to SEQ ID NO: 49 and the immunoglobulin heavy chain comprises an amino acid sequence with at least 90% identity to SEQ ID NO: 50.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; and the immunoglobulin light chain comprises an amino acid sequence with at least 90% identity to SEQ ID NO: 51 and the immunoglobulin heavy chain comprises an amino acid sequence with at least 90% identity to SEQ ID NO: 52.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; and the immunoglobulin light chain comprises an amino acid sequence with at least 90% identity to SEQ ID NO: 55 and the immunoglobulin heavy chain comprises an amino acid sequence with at least 90% identity to SEQ ID NO: 56.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; and the immunoglobulin light chain comprises an amino acid sequence with at least 90% identity to SEQ ID NO: 57 and the immunoglobulin heavy chain comprises an amino acid sequence with at least 90% identity to SEQ ID NO: 58.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO:21; and the immunoglobulin light chain comprises an amino acid sequence with at least 90% identity to SEQ ID NO: 59 and the immunoglobulin heavy chain comprises an amino acid sequence with at least 90% identity to SEQ ID NO: 60.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; and the immunoglobulin light chain comprises an amino acid sequence with at least 90% identity to SEQ ID NO: 61 and the immunoglobulin heavy chain comprises an amino acid sequence with at least 90% identity to SEQ ID NO: 62.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; and the immunoglobulin light chain comprises an amino acid sequence with at least 90% identity to SEQ ID NO: 67 and the immunoglobulin heavy chain comprises an amino acid sequence with at least 90% identity to SEQ ID NO: 68.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; and the immunoglobulin light chain comprises an amino acid sequence with at least 90% identity to SEQ ID NO: 69 and the immunoglobulin heavy chain comprises an amino acid sequence with at least 90% identity to SEQ ID NO: 70.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and the immunoglobulin light chain comprises an amino acid sequence with at least 90% identity to SEQ ID NO: 71 and the immunoglobulin heavy chain comprises an amino acid sequence with at least 90% identity to SEQ ID NO: 72.

In some embodiments, the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the amino acid sequences comprise CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28; and the immunoglobulin light chain comprises an amino acid sequence with at least 90% identity to SEQ ID NO: 73 and the immunoglobulin heavy chain comprises an amino acid sequence with at least 90% identity to SEQ ID NO: 74.

In some embodiments, the TROP2 binding domain has a faster off rate (larger k_{diss}) for TROP2 binding as compared to a TROP2 binding domain that comprises an immunoglobulin light chain according to SEQ ID NO: 29 and an immunoglobulin heavy chain according to SEQ ID NO: 30 as measured under substantially similar kinetic assay conditions. In some embodiments, the TROP2 binding domain has weaker binding to TROP2 as compared to a TROP2 binding domain that comprises an immunoglobulin light chain according to SEQ ID NO: 29 and an immunoglobulin heavy chain according to SEQ ID NO: 30 as measured by enzyme-linked immunosorbent assay (ELISA) in substantially similar assay conditions. In some embodiments, the TROP2 binding domain has an increased EC₅₀ for TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions. In some embodiments, the TROP2 comprises an amino acid sequence according to SEQ ID NO: 100 (see Table 10).

### CD3 Binding Domain

In some embodiments, the recombinant antibody or antigen binding fragment further comprises a CD3 binding domain. In some embodiments, the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 75, wherein CDR2-L comprises the amino acid sequence of SEQ ID NO: 76, wherein CDR3-L comprises the amino acid sequence of SEQ ID NO: 77, wherein CDR1-H comprises the amino acid sequence of SEQ ID NO: 78, wherein CDR2-H comprises the amino acid sequence of SEQ ID NO: 79, and wherein CDR3-H comprises the amino acid sequence of SEQ ID NO: 80.

In some embodiments, the CD3 binding domain comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H; wherein CDR1-L comprises the amino acid sequence of SEQ ID NO: 75; wherein CDR2-L comprises the amino acid sequence of SEQ ID NO: 76; wherein CDR3-L comprises the amino acid sequence of Z₁-Z₂-W-Z₃-Z₄-Z₅-Z₆-W-Z₇-Z₈; wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 78; wherein CDR2-H comprises the amino acid sequence of SEQ ID NO: 79; wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁; wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₁ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A. In some embodiments, Z₁ is V, G, L, I, or A; Z₂ is L, V, I, or A; Z₃ is Y, W, F, or A; Z₄ is S, G, T, or A; Z₅ is N, Q, D, E, or A; Z₆ is R, K, or A; Z₇ is V, G, L, I, or A; Z₈ is F, Y, W, or A; Z₉ is V, G, L, I, or A; Z₁₀ is R, K, or A; Z₁₂ is G, S, T, or A; Z₁₃ is F, Y, W, or A; Z₁₄ is G, S, T, or A; Z₁₅ is N, Q, D, E, or A; Z₁₆ is S, G, T, or A; Z₁₇ is Y, W, F, or A; Z₁₈ is I, V, L, or A; Z₉ is S, G, T, or A; Z₂₀ is W, Y, F, or A; and Z₂₁ is Y, W, F, or A. In some embodiments, Z₈ is F. In some embodiments, Z₁₀ is R; Z₁₁ is H; Z₁₃ is F; Z₁₈ is I; Z₁₉ is S; and Z₂₀ is W.

In some embodiments, CDR3-L comprises an amino acid sequence selected from SEQ ID NOs: 77, 105-106, 108-111, and 113-114. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; and CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; and CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80.

In some embodiments, CDR3-H comprises an amino acid sequence selected from SEQ ID NOs: 80, 116-119, 121-127, and 129-130. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130. In some embodiments, the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

In some embodiments, the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 107, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 112, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 120; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 128; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

In some embodiments, the immunoglobulin light chain of the CD3 binding domain comprises a variable domain of an immunoglobulin kappa (IgK) or immunoglobulin lambda (IgL) light chain. In some embodiments, the immunoglobulin heavy chain of the CD3 binding domain comprises a variable domain of an IgG1, IgG2, IgG3, or IgG4 heavy chain.

TABLE 4
CD3 binding domain light chain complementarity
determining regions (CDRs) as based on
IMGT CDR numbering system.

| Construct | Amino Acid | |
|---|---|---|
| Description | Sequence (N to C) | SEQ ID NO: |
| CDR1-L | TGAVTSGNY | 75 |
| CDR2-L | GT | 76 |
| CDR3-L | VLWYSNRWV | 77 |
| CDR1-L | TGAVTSANY | 104 |
| CDR3-L V231A | ALWYSNRWV | 105 |
| CDR3-L L232A | VAWYSNRWV | 106 |
| CDR3-L W233A | VLAYSNRWV | 107 |
| CDR3-L Y234A | VLWASNRWV | 108 |
| CDR3-L S235A | VLWYANRWV | 109 |
| CDR3-L N236A | VLWYSARWV | 110 |
| CDR3-L R237A | VLWYSNAWV | 111 |
| CDR3-L W238A | VLWYSNRAV | 112 |
| CDR3-L V239A | VLWYSNRWA | 113 |
| CDR3-L F240A | VLWYSNRWVA | 114 |

TABLE 5
CD3 binding domain heavy chain complementarity
determining regions (CDRs) as based on
IMGT CDR numbering system.

| Construct | Amino Acid | |
|---|---|---|
| Description | Sequence (N to C) | SEQ ID NO: |
| CDR1-H | GFTFNKYA | 78 |
| CDR2-H | IRSKYNNYAT | 79 |
| CDR3-H | VRHGNFGNSYISYWAY | 80 |
| CDR1-H | GFTFQKYA | 115 |
| CDR3-H V99A | ARHGNFGNSYISYWAY | 116 |
| CDR3-H R100A | VAHGNFGNSYISYWAY | 117 |
| CDR3-H H101A | VRAGNFGNSYISYWAY | 118 |
| CDR3-H G102A | VRHANFGNSYISYWAY | 119 |
| CDR3-H N103A | VRHGAFGNSYISYWAY | 120 |
| CDR3-H F104A | VRHGNAGNSYISYWAY | 121 |
| CDR3-H G105A | VRHGNFANSYISYWAY | 122 |
| CDR3-H N106A | VRHGNFGASYISYWAY | 123 |
| CDR3-H S107A | VRHGNFGNAYISYWAY | 124 |
| CDR3-H Y108A | VRHGNFGNSAISYWAY | 125 |
| CDR3-H I109A | VRHGNFGNSYASYWAY | 126 |
| CDR3-H S110A | VRHGNFGNSYIAYWAY | 127 |
| CDR3-H Y111A | VRHGNFGNSYISAWAY | 128 |
| CDR3-H W112A | VRHGNFGNSYISYAAY | 129 |
| CDR3-H Y114A | VRHGNFGNSYISYWAA | 130 |

In some embodiments, the immunoglobulin light chain of the CD3 binding domain comprises an amino acid sequence with at least 90%, 95%, 96%, 97%, 98%, 99%, or 100% identity to SEQ ID NO: 81. In some embodiments, the immunoglobulin heavy chain of the CD3 binding domain comprises an amino acid sequence with at least 90%, 95%, 96%, 97%, 98%, 99%, or 100% identity to SEQ ID NO: 82. In some embodiments, the immunoglobulin light chain of the CD3 binding domain comprises an amino acid sequence with at least 90%, 95%, 96%, 97%, 98%, 99%, or 100% identity to SEQ ID NO: 81 and the immunoglobulin heavy chain of the CD3 binding domain comprises an amino acid sequence with at least 90%, 95%, 96%, 97%, 98%, 99%, or 100% identity to SEQ ID NO: 82. In some embodiments, the CD3 binding domain comprises an amino acid sequence with at least 90%, 95%, 96%, 97%, 98%, 99%, or 100% identity to SEQ ID NO: 99. In some embodiments, the CD3 binding domain comprises an amino acid sequence with at least 90% identity to SEQ ID NO: 99. In some embodiments, the CD3 binding domain comprises a Fab, Fab′, (Fab′)₂ or a single chain variable fragment (scFv). In some embodiments, the CD3 binding domain is the scFv.

In some embodiments, the CD3 binding domain comprises an amino acid sequence with at least 90%, 95%, 96%, 97%, 98%, 99%, or 100% identity to SEQ ID NO: 131. In some embodiments, the CD3 binding domain comprises an amino acid sequence with at least 90%, 95%, 96%, 97%, 98%, 99%, or 100% identity to SEQ ID NO: 132. In some embodiments, the CD3 binding domain comprises an amino acid sequence with at least 90%, 95%, 96%, 97%, 98%, 99%, or 100% identity to SEQ ID NO: 133. In some embodiments, the CD3 binding domain comprises an amino acid sequence with at least 90%, 95%, 96%, 97%, 98%, 99%, or 100% identity to SEQ ID NO: 134. In some embodiments, the CD3 binding domain comprises an amino acid sequence with at least 90%, 95%, 96%, 97%, 98%, 99%, or 100% identity to SEQ ID NO: 135. In some embodiments, the CD3 binding domain comprises an amino acid sequence with at least 90%, 95%, 96%, 97%, 98%, 99%, or 100% identity to SEQ ID NO: 136. In some embodiments, the CD3 binding domain comprises an amino acid sequence with at least 90%, 95%, 96%, 97%, 98%, 99%, or 100% identity to SEQ ID NO: 137. In some embodiments, the CD3 binding domain comprises an amino acid sequence with at least 90%, 95%, 96%, 97%, 98%, 99%, or 100% identity to SEQ ID NO: 138. In some embodiments, the CD3 binding domain comprises an amino acid sequence with at least 90%, 95%, 96%, 97%, 98%, 99%, or 100% identity to SEQ ID NO: 139. In some embodiments, the CD3 binding domain comprises an amino acid sequence with at least 90%, 95%, 96%, 97%, 98%, 99%, or 100% identity to SEQ ID NO: 140. In some embodiments, the CD3 binding domain comprises an amino acid sequence with at least 90%, 95%, 96%, 97%, 98%, 99%, or 100% identity to SEQ ID NO: 141. In some embodiments, the CD3 binding domain comprises an amino acid sequence with at least 90%, 95%, 96%, 97%, 98%, 99%, or 100% identity to SEQ ID NO: 142. In some embodiments, the CD3 binding domain comprises an amino acid sequence with at least 90%, 95%, 96%, 97%, 98%, 99%, or 100% identity to SEQ ID NO: 143. In some embodiments, the CD3 binding domain comprises an amino acid sequence with at least 90%, 95%, 96%, 97%, 98%, 99%, or 100% identity to SEQ ID NO: 144. In some embodiments, the CD3 binding domain comprises an amino acid sequence with at least 90%, 95%, 96%, 97%, 98%, 99%, or 100% identity to SEQ ID NO: 145. In some embodiments, the CD3 binding domain comprises an amino acid sequence with at least 90%, 95%, 96%, 97%, 98%, 99%, or 100% identity to SEQ ID NO: 146. In some embodiments, the CD3 binding domain comprises an amino acid sequence with at least 90%, 95%, 96%, 97%, 98%, 99%, or 100% identity to SEQ ID NO: 147. In some embodiments, the CD3 binding domain comprises an amino acid sequence with at least 90%, 95%, 96%, 97%, 98%, 99%, or 100% identity to SEQ ID NO: 148. In some embodiments, the CD3 binding domain comprises an amino acid sequence with at least 90%, 95%, 96%, 97%, 98%, 99%, or 100% identity to SEQ ID NO: 149. In some embodiments, the CD3 binding domain comprises an amino acid sequence with at least 90%, 95%, 96%, 97%, 98%, 99%, or 100% identity to SEQ ID NO: 150. In some embodiments, the CD3 binding domain comprises an amino acid sequence with at least 90%, 95%, 96%, 97%, 98%, 99%, or 100% identity to SEQ ID NO: 151. In some embodiments, the CD3 binding domain comprises an amino acid sequence with at least 90%, 95%, 96%, 97%, 98%, 99%, or 100% identity to SEQ ID NO: 152. In some embodiments, the CD3 binding domain comprises an amino acid sequence with at least 90%, 95%, 96%, 97%, 98%, 99%, or 100% identity to SEQ ID NO: 153. In some embodiments, the CD3 binding domain comprises an amino acid sequence with at least 90%, 95%, 96%, 97%, 98%, 99%, or 100% identity to SEQ ID NO: 154. In some embodiments, the CD3 binding domain comprises an amino acid sequence with at least 90%, 95%, 96%, 97%, 98%, 99%, or 100% identity to SEQ ID NO: 155.

TABLE 6
CD3 heavy chain and light chain sequences and scFvs

| | | SEQ |
|---|---|---|
| | | ID |
| Construct Description | Amino Acid Sequence (N to C) | NO: |
| CD3 light chain | QTVVTQEPSLTVSPGGTVTLTCGSSTGAVTSGNYPNW | 81 |
| | VQQKPGQAPRGLIGGTKFLAPGTPARFSGSLLGGKAA | |
| | LTLSGVQPEDEAEYYCVLWYSNRWVFGGGTKLTVL | |
| CD3 heavy chain | EVQLVESGGGLVQPGGSLKLSCAASGFTFNKYAMNW | 82 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSS | |
| CD3 scFv (SP34.185) | EVQLVESGGGLVQPGGSLKLSCAASGFTFNKYAMNW | 99 |
| | VRQAPGKGLEWVARIRSKYNNYATYYADSVKDRFTI | |
| | SRDDSKNTAYLQMNNLKTEDTAVYYCVRHGNFGNS | |
| | YISYWAYWGQGTLVTVSSGGGGSGGGGSGGGGSQTV | |
| | VTQEPSLTVSPGGTVTLTCGSSTGAVTSGNYPNWVQQ | |
| | KPGQAPRGLIGGTKFLAPGTPARFSGSLLGGKAALTLS | |
| | GVQPEDEAEYYCVLWYSNRWVFGGGTKLTVL | |
| CD3 scFv V99A | EVQLVESGGGLVQPGGSLKLSCAASGFTFNKYAMNW | 131 |
| | VRQAPGKGLEWVARIRSKYNNYATYYADSVKDRFTIS | |
| | RDDSKNTAYLQMNNLKTEDTAVYYCARHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv R100A | EVQLVESGGGLVQPGGSLKLSCAASGFTFNKYAMNW | 132 |
| | VRQAPGKGLEWVARIRSKYNNYATYYADSVKDRFTIS | |
| | RDDSKNTAYLQMNNLKTEDTAVYYCVAHGNFGNSYI | |
| | SYWAYWGQGTLVTVSSGGGGGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv H101A | EVQLVESGGGLVQPGGSLKLSCAASGFTFNKYAMNW | 133 |
| | VRQAPGKGLEWVARIRSKYNNYATYYADSVKDRFTIS | |
| | RDDSKNTAYLQMNNLKTEDTAVYYCVRAGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv G102A | EVQLVESGGGLVQPGGSLKLSCAASGFTFNKYAMNW | 134 |
| | VRQAPGKGLEWVARIRSKYNNYATYYADSVKDRFTIS | |
| | RDDSKNTAYLQMNNLKTEDTAVYYCVRHANFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv N103A | EVQLVESGGGLVQPGGSLKLSCAASGFTFNKYAMNW | 135 |
| | VRQAPGKGLEWVARIRSKYNNYATYYADSVKDRFTIS | |
| | RDDSKNTAYLQMNNLKTEDTAVYYCVRHGAFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv F104A | EVQLVESGGGLVQPGGSLKLSCAASGFTFNKYAMNW | 136 |
| | VRQAPGKGLEWVARIRSKYNNYATYYADSVKDRFTIS | |
| | RDDSKNTAYLQMNNLKTEDTAVYYCVRHGNAGNSY | |
| | ISYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv G105A | EVQLVESGGGLVQPGGSLKLSCAASGFTFNKYAMNW | 137 |
| | VRQAPGKGLEWVARIRSKYNNYATYYADSVKDRFTIS | |
| | RDDSKNTAYLQMNNLKTEDTAVYYCVRHGNFANSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv N106A | EVQLVESGGGLVQPGGSLKLSCAASGFTFNKYAMNW | 138 |
| | VRQAPGKGLEWVARIRSKYNNYATYYADSVKDRFTIS | |
| | RDDSKNTAYLQMNNLKTEDTAVYYCVRHGNFGASYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv S107A | EVQLVESGGGLVQPGGSLKLSCAASGFTFNKYAMNW | 139 |
| | VRQAPGKGLEWVARIRSKYNNYATYYADSVKDRFTIS | |
| | RDDSKNTAYLQMNNLKTEDTAVYYCVRHGNFGNAY | |
| | ISYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv Y108A | EVQLVESGGGLVQPGGSLKLSCAASGFTFNKYAMNW | 140 |
| | VRQAPGKGLEWVARIRSKYNNYATYYADSVKDRFTIS | |
| | RDDSKNTAYLQMNNLKTEDTAVYYCVRHGNFGNSAI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv I109A | EVQLVESGGGLVQPGGSLKLSCAASGFTFNKYAMNW | 141 |
| | VRQAPGKGLEWVARIRSKYNNYATYYADSVKDRFTIS | |
| | RDDSKNTAYLQMNNLKTEDTAVYYCVRHGNFGNSY | |
| | ASYWAYWGQGTLVTVSSGGGGSGGGGSGGGGSQTV | |
| | VTQEPSLTVSPGGTVTLTCGSSTGAVTSGNYPNWVQQ | |
| | KPGQAPRGLIGGTKFLAPGTPARFSGSLLGGKAALTLS | |
| | GVQPEDEAEYYCVLWYSNRWVFGGGTKLTVL | |
| CD3 scFv S110A | EVQLVESGGGLVQPGGSLKLSCAASGFTFNKYAMNW | 142 |
| | VRQAPGKGLEWVARIRSKYNNYATYYADSVKDRFTIS | |
| | RDDSKNTAYLQMNNLKTEDTAVYYCVRHGNFGNSYI | |
| | AYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv Y111A | EVQLVESGGGLVQPGGSLKLSCAASGFTFNKYAMNW | 143 |
| | VRQAPGKGLEWVARIRSKYNNYATYYADSVKDRFTIS | |
| | RDDSKNTAYLQMNNLKTEDTAVYYCVRHGNFGNSYI | |
| | SAWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv W112A | EVQLVESGGGLVQPGGSLKLSCAASGFTFNKYAMNW | 144 |
| | VRQAPGKGLEWVARIRSKYNNYATYYADSVKDRFTIS | |
| | RDDSKNTAYLQMNNLKTEDTAVYYCVRHGNFGNSYI | |
| | SYAAYWGQGTLVTVSSGGGGSGGGGSGGGGSQTVVT | |
| | QEPSLTVSPGGTVTLTCGSSTGAVTSGNYPNWVQQKP | |
| | GQAPRGLIGGTKFLAPGTPARFSGSLLGGKAALTLSGV | |
| | QPEDEAEYYCVLWYSNRWVFGGGTKLTVL | |
| CD3 scFv Y114A | EVQLVESGGGLVQPGGSLKLSCAASGFTFNKYAMNW | 145 |
| | VRQAPGKGLEWVARIRSKYNNYATYYADSVKDRFTIS | |
| | RDDSKNTAYLQMNNLKTEDTAVYYCVRHGNFGNSYI | |
| | SYWAAWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVFGGGTKLTVL | |
| CD3 scFv V231A | EVQLVESGGGLVQPGGSLKLSCAASGFTFNKYAMNW | 146 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCALWYSNRWVFGGGTKLTVL | |
| CD3 scFv L232A | EVQLVESGGGLVQPGGSLKLSCAASGFTFNKYAMNW | 147 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVAWYSNRWVFGGGTKLTVL | |
| CD3 scFv W233A | EVQLVESGGGLVQPGGSLKLSCAASGFTFNKYAMNW | 148 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLAYSNRWVFGGGTKLTVL | |
| CD3 scFv Y234A | EVQLVESGGGLVQPGGSLKLSCAASGFTFNKYAMNW | 149 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWASNRWVFGGGTKLTVL | |
| CD3 scFv S235A | EVQLVESGGGLVQPGGSLKLSCAASGFTFNKYAMNW | 150 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYANRWVFGGGTKLTVL | |
| CD3 scFv N236A | EVQLVESGGGLVQPGGSLKLSCAASGFTFNKYAMNW | 151 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSARWVFGGGTKLTVL | |
| CD3 scFv R237A | EVQLVESGGGLVQPGGSLKLSCAASGFTFNKYAMNW | 152 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNAWVFGGGTKLTVL | |
| CD3 scFv W238A | EVQLVESGGGLVQPGGSLKLSCAASGFTFNKYAMNW | 153 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRAVFGGGTKLTVL | |
| CD3 scFv V239A | EVQLVESGGGLVQPGGSLKLSCAASGFTFNKYAMNW | 154 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWAFGGGTKLTVL | |
| CD3 scFv F240A | EVQLVESGGGLVQPGGSLKLSCAASGFTFNKYAMNW | 155 |
| | VRQAPGKGLEWVARIRSKYNNYATYYADSVKDRFTIS | |
| | RDDSKNTAYLQMNNLKTEDTAVYYCVRHGNFGNSYI | |
| | SYWAYWGQGTLVTVSSGGGGSGGGGSGGGGSQTVV | |
| | TQEPSLTVSPGGTVTLTCGSSTGAVTSGNYPNWVQQK | |
| | PGQAPRGLIGGTKFLAPGTPARFSGSLLGGKAALTLSG | |
| | VQPEDEAEYYCVLWYSNRWVAGGGTKLTVL | |
| CD3 scFv | EVQLVESGGGLVQPGGSLKLSCAASGFTFNKYAMNW | 156 |
| (mutations relative to SEQ ID | VRQASGKGLEWVGRIRSKYNNYATYYADSVKDRFTIS | |
| NO: 99: P41S, A49G, N87S, | RDDSKNTAYLQMNSLKTEDTAVYYCVRHGNFGNSYI | |
| L150F, T151S, G163R, P175A, | SYWAYWGQGTLVTVSSGGGGSGGGGSGGGGSQTVV | |
| K181T, T200V, A202D, L208I, | TQEPSFSVSPGGTVTLTCRSSTGAVTSGNYANWVQQT | |
| G211N, L2171, S218T, V220A, | PGQAPRGLIGGTKFLAPGVPDRFSGSILGNKAALTITGA | |
| P222A, E223D, A226S, E227D) | QADDESDYYCVLWYSNRWVFGGGTKLTVL | |
| CD3 scFv | EVQLVESGGGLVQPGGSLKLSCAASGFTFQKYAMNW | 157 |
| (mutations relative to SEQ ID | VRQAPGKGLEWVARIRSKYNNYATYYADSVKDRFTIS | |
| NO: 99: N30Q, I109V, G172A, | RDDSKNTAYLQMNNLKTEDTAVYYCVRHGNFGNSY | |
| V231A) | VSYWAYWGQGTLVTVSSGGGGSGGGGSGGGGSQTV | |
| | VTQEPSLTVSPGGTVTLTCGSSTGAVTSANYPNWVQQ | |
| | KPGQAPRGLIGGTKFLAPGTPARFSGSLLGGKAALTLS | |
| | GVQPEDEAEYYCALWYSNRWVFGGGTKLTVL | |

In some embodiments, the N-term of the immunoglobulin heavy chain of the TROP2 binding domain is bound to the C-term of the immunoglobulin light chain of the CD3 binding domain. In some embodiments, the C-term of the immunoglobulin heavy chain of the TROP2 binding domain is bound to the N-term of the immunoglobulin light chain of the CD3 binding domain. In some embodiments, the N-term of the immunoglobulin heavy chain of the TROP2 binding domain is bound to the C-term of the immunoglobulin heavy chain of the CD3 binding domain. In some embodiments, the C-term of the immunoglobulin heavy chain of the TROP2 binding domain is bound to the N-term of the immunoglobulin heavy chain of the CD3 binding domain. In some embodiments, the N-term of the immunoglobulin light chain of the TROP2 binding domain is bound to the C-term of the immunoglobulin light chain of the CD3 binding domain. In some embodiments, the C-term of the immunoglobulin light chain of the TROP2 binding domain is bound to the N-term of the immunoglobulin light chain of the CD3 binding domain. In some embodiments, the N-term of the immunoglobulin light chain of the TROP2 binding domain is bound to the C-term of the immunoglobulin heavy chain of the CD3 binding domain. In some embodiments, the C-term of the immunoglobulin light chain of the TROP2 binding domain is bound to the N-term of the immunoglobulin heavy chain of the CD3 binding domain.

In some embodiments, the CD3 binding domain comprises a Fab, Fab′, (Fab′)₂ or a single chain variable fragment (scFv). In some embodiments, the CD3 binding domain is the scFv. In some embodiments, the CD3 binding domain is a scFv and the TROP2 binding domain is a Fab or Fab′. In some embodiments, the scFv is bound to the immunoglobulin heavy chain of the Fab or Fab′. In some embodiments, the scFv is bound to the immunoglobulin light chain of the Fab or Fab′. In some embodiments, the immunoglobulin light chain of the scFv is bound to the immunoglobulin heavy chain of the Fab or Fab′. In some embodiments, the immunoglobulin light chain of the scFv is bound to the immunoglobulin light chain of the Fab or Fab′. In some embodiments, the immunoglobulin heavy chain of the scFv is bound to the immunoglobulin heavy chain of the Fab or Fab′.

### T Cell Engager Sequences

In some embodiments, the recombinant antibody or antigen binding fragment thereof comprises amino acid sequences with at least 90%, 95%, 96%, 97%, 98%, 99%, or 100% identity to SEQ ID NO: 87 and SEQ ID NO: 88. In some embodiments, the recombinant antibody or antigen binding fragment thereof comprises amino acid sequences with at least 90%, 95%, 96%, 97%, 98%, 99%, or 100% identity to SEQ ID NO: 89 and SEQ ID NO: 90. In some embodiments, the recombinant antibody or antigen binding fragment thereof comprises amino acid sequences with at least 90%, 95%, 96%, 97%, 98%, 99%, or 100% identity to SEQ ID NO: 91 and SEQ ID NO: 92. In some embodiments, the recombinant antibody or antigen binding fragment thereof comprises amino acid sequences with at least 90%, 95%, 96%, 97%, 98%, 99%, or 100% identity to SEQ ID NO: 93 and SEQ ID NO: 94. In some embodiments, the recombinant antibody or antigen binding fragment thereof comprises amino acid sequences with at least 90%, 95%, 96%, 97%, 98%, 99%, or 100% identity to SEQ ID NO: 95 and SEQ ID NO: 96. In some embodiments, the recombinant antibody or antigen binding fragment thereof comprises amino acid sequences with at least 90%, 95%, 96%, 97%, 98%, 99%, or 100% identity to SEQ ID NO: 97 and SEQ ID NO: 98.

In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 188. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 189. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 190. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 194. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 195. In some embodiments, the recombinant antibody or antigen binding fragment thereof comprises amino acid sequences with at least 90%, 95%, 96%, 97%, 98%, 99%, or 100% identity to SEQ ID NO: 158 and SEQ ID NO: 196.

In some embodiments, the recombinant antibody or antigen binding fragment thereof has weaker cytotoxicity activity as compared to a recombinant antibody or antigen binding fragment thereof that comprises an immunoglobulin light chain according to SEQ ID NO: 83 or 85 and an immunoglobulin heavy chain according to SEQ ID NO: 84 or 86 as measured in an in vitro tumor cell killing assay under substantially similar assay conditions.

In some embodiments, the recombinant antibodies or antigen binding fragments thereof comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₂, or P₁ₐ or Pi, P₂, and P₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to the recombinant antibodies or antigen binding fragments thereof including the peptide backbone, the amino acid side chains, and the terminus.

TABLE 7
TROP2- T cell engager (TCE) sequences

| Construct Description | Amino Acid Sequence (N to C) | SEQ ID NO: |
|---|---|---|
| TCE-1 LC | DIQLTQSPSSLSASVGDRVSITCKASQDVSI | 83 |
| TROP2 Fab × SP34.185 scFv | AVAWYQQKPGKAPKLLIYSASYRYTGVP | |
| V1: anti-TROP2 Fab LC | DRFSGSGSGTDFTLTISSLQPEDFAVYYCQ | |
| Vh: SP34.185 × anti-TROP2 Fab | QHYITPLTFGAGTKVEIKRTVAAPSVFIFPP | |
| HC | SDEQLKSGTASVVCLLNNFYPREAKVQW | |
| | KVDNALQSGNSQESVTEQDSKDSTYSLSS | |
| | TLTLSKADYEKHKVYACEVTHQGLSSPVT | |
| | KSFNRGEC | |
| TCE-1 HC | EVQLVESGGGLVQPGGSLKLSCAASGFTF | 84 |
| TROP2 Fab × SP34.185 scFv | NKYAMNWVRQAPGKGLEWVARIRSKYN | |
| V1: anti-TROP2 Fab LC | NYATYYADSVKDRFTISRDDSKNTAYLQ | |
| Vh: SP34.185 × anti-TROP2 Fab | MNNLKTEDTAVYYCVRHGNFGNSYISYW | |
| HC | AYWGQGTLVTVSSGGGGSGGGGSGGGGS | |
| | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSQ | |
| | VQLQQSGSELKKPGASVKVSCKASGYTFT | |
| | NYGMNWVKQAPGQGLKWMGWINTYTG | |
| | EPTYTDDFKGRFAFSLDTSVSTAYLQISSL | |
| | KADDTAVYFCARGGFGSSYWYFDVWGQ | |
| | GSLVTVSSASTKGPSVFPLAPSSKSTSGGT | |
| | AALGCLVKDYFPEPVTVSWNSGALTSGVH | |
| | TFPAVLQSSGLYSLSSVVTVPSSSLGTQTYI | |
| | CNVNHKPSNTKVDKKVEPKSC | |
| TCE-2 LC | EVQLVESGGGLVQPGGSLKLSCAASGFTF | 85 |
| TROP2 Fab × SP34.185 scFv | NKYAMNWVRQAPGKGLEWVARIRSKYN | |
| V1: SP34.185 scFv × anti- | NYATYYADSVKDRFTISRDDSKNTAYLQ | |
| TROP2 Fab LC | MNNLKTEDTAVYYCVRHGNFGNSYISYW | |
| Vh: anti-TROP2 Fab HC | AYWGQGTLVTVSSGGGGSGGGGSGGGGS | |
| AviTag | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSDI | |
| | QLTQSPSSLSASVGDRVSITCKASQDVSIA | |
| | VAWYQQKPGKAPKLLIYSASYRYTGVPD | |
| | RFSGSGSGTDFTLTISSLQPEDFAVYYCQQ | |
| | HYITPLTFGAGTKVEIKRTVAAPSVFIFPPS | |
| | DEQLKSGTASVVCLLNNFYPREAKVQWK | |
| | VDNALQSGNSQESVTEQDSKDSTYSLSSTL | |
| | TLSKADYEKHKVYACEVTHQGLSSPVTKS | |
| | FNRGEC | |
| TCE-2 HC | QVQLQQSGSELKKPGASVKVSCKASGYTF | 86 |
| TROP2 Fab × SP34.185 scFv | TNYGMNWVKQAPGQGLKWMGWINTYT | |
| V1: SP34.185 scFv × anti- | GEPTYTDDFKGRFAFSLDTSVSTAYLQISS | |
| TROP2 Fab LC | LKADDTAVYFCARGGFGSSYWYFDVWG | |
| Vh: anti-TROP2 Fab HC | QGSLVTVSSASTKGPSVFPLAPSSKSTSGG | |
| AviTag | TAALGCLVKDYFPEPVTVSWNSGALTSGV | |
| | HTFPAVLQSSGLYSLSSVVTVPSSSLGTQT | |
| | YICNVNHKPSNTKVDKKVEPKSC | |
| TCE-3 LC | EVQLVESGGGLVQPGGSLKLSCAASGFTF | 87 |
| TROP2 Fab (HC R98A) TCE | NKYAMNWVRQAPGKGLEWVARIRSKYN | |
| V1: SP34.185 scFv + TROP2 | NYATYYADSVKDRFTISRDDSKNTAYLQ | |
| Fab LC | MNNLKTEDTAVYYCVRHGNFGNSYISYW | |
| Vh: TROP2 Fab HC (R98A) | AYWGQGTLVTVSSGGGGSGGGGSGGGGS | |
| HisTag AviTag | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSDI | |
| | QLTQSPSSLSASVGDRVSITCKASQDVSIA | |
| | VAWYQQKPGKAPKLLIYSASYRYTGVPD | |
| | RFSGSGSGTDFTLTISSLQPEDFAVYYCQQ | |
| | HYITPLTFGAGTKVEIKRTVAAPSVFIFPPS | |
| | DEQLKSGTASVVCLLNNFYPREAKVQWK | |
| | VDNALQSGNSQESVTEQDSKDSTYSLSSTL | |
| | TLSKADYEKHKVYACEVTHQGLSSPVTKS | |
| | FNRGEC | |
| TCE-3 HC | QVQLQQSGSELKKPGASVKVSCKASGYTF | 88 |
| TROP2 Fab (HC R98A) TCE | TNYGMNWVKQAPGQGLKWMGWINTYT | |
| V1: SP34.185 scFv + TROP2 | GEPTYTDDFKGRFAFSLDTSVSTAYLQISS | |
| Fab LC | LKADDTAVYFCAAGGFGSSYWYFDVWG | |
| Vh: TROP2 Fab HC (R98A) | QGSLVTVSSASTKGPSVFPLAPSSKSTSGG | |
| HisTag AviTag | TAALGCLVKDYFPEPVTVSWNSGALTSGV | |
| | HTFPAVLQSSGLYSLSSVVTVPSSSLGTQT | |
| | YICNVNHKPSNTKVDKKVEPKSC | |
| TCE-4 LC | EVQLVESGGGLVQPGGSLKLSCAASGFTF | 89 |
| TROP2 Fab (HC F108A) TCE | NKYAMNWVRQAPGKGLEWVARIRSKYN | |
| V1: SP34.185 scFv + TROP2 | NYATYYADSVKDRFTISRDDSKNTAYLQ | |
| Fab LC | MNNLKTEDTAVYYCVRHGNFGNSYISYW | |
| Vh: TROP2 Fab HC (F108A) | AYWGQGTLVTVSSGGGGSGGGGSGGGGS | |
| His Tag AviTag | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSDI | |
| | QLTQSPSSLSASVGDRVSITCKASQDVSIA | |
| | VAWYQQKPGKAPKLLIYSASYRYTGVPD | |
| | RFSGSGSGTDFTLTISSLQPEDFAVYYCQQ | |
| | HYITPLTFGAGTKVEIKRTVAAPSVFIFPPS | |
| | DEQLKSGTASVVCLLNNFYPREAKVQWK | |
| | VDNALQSGNSQESVTEQDSKDSTYSLSSTL | |
| | TLSKADYEKHKVYACEVTHQGLSSPVTKS | |
| | FNRGEC | |
| TCE-4 HC | QVQLQQSGSELKKPGASVKVSCKASGYTF | 90 |
| TROP2 Fab (HC F108A) TCE | TNYGMNWVKQAPGQGLKWMGWINTYT | |
| V1: SP34.185 scFv + TROP2 | GEPTYTDDFKGRFAFSLDTSVSTAYLQISS | |
| Fab LC | LKADDTAVYFCARGGFGSSYWYADVWG | |
| Vh: TROP2 Fab HC (F108A) | QGSLVTVSSASTKGPSVFPLAPSSKSTSGG | |
| His Tag AviTag | TAALGCLVKDYFPEPVTVSWNSGALTSGV | |
| | HTFPAVLQSSGLYSLSSVVTVPSSSLGTQT | |
| | YICNVNHKPSNTKVDKKVEPKSC | |
| TCE-5 LC | EVQLVESGGGLVQPGGSLKLSCAASGFTF | 91 |
| TROP2 Fab (HC D109A) TCE | NKYAMNWVRQAPGKGLEWVARIRSKYN | |
| V1: SP34.185 scFv + TROP2 | NYATYYADSVKDRFTISRDDSKNTAYLQ | |
| Fab LC | MNNLKTEDTAVYYCVRHGNFGNSYISYW | |
| Vh: TROP2 Fab HC (D109A) | AYWGQGTLVTVSSGGGGSGGGGSGGGGS | |
| HisTag AviTag | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSDI | |
| | QLTQSPSSLSASVGDRVSITCKASQDVSIA | |
| | VAWYQQKPGKAPKLLIYSASYRYTGVPD | |
| | RFSGSGSGTDFTLTISSLQPEDFAVYYCQQ | |
| | HYITPLTFGAGTKVEIKRTVAAPSVFIFPPS | |
| | DEQLKSGTASVVCLLNNFYPREAKVQWK | |
| | VDNALQSGNSQESVTEQDSKDSTYSLSSTL | |
| | TLSKADYEKHKVYACEVTHQGLSSPVTKS | |
| | FNRGEC | |
| TCE-5 HC | QVQLQQSGSELKKPGASVKVSCKASGYTF | 92 |
| TROP2 Fab (HC D109A) TCE | TNYGMNWVKQAPGQGLKWMGWINTYT | |
| V1: SP34.185 scFv + TROP2 | GEPTYTDDFKGRFAFSLDTSVSTAYLQISS | |
| Fab LC | LKADDTAVYFCARGGFGSSYWYFAVWG | |
| Vh: TROP2 Fab HC (D109A) | QGSLVTVSSASTKGPSVFPLAPSSKSTSGG | |
| HisTag AviTag | TAALGCLVKDYFPEPVTVSWNSGALTSGV | |
| | HTFPAVLQSSGLYSLSSVVTVPSSSLGTQT | |
| | YICNVNHKPSNTKVDKKVEPKSC | |
| TCE-6 LC | DIQLTQSPSSLSASVGDRVSITCKASQDVSI | 93 |
| TROP2 Fab (HC R98A) TCE | AVAWYQQKPGKAPKLLIYSASYRYTGVP | |
| V1: TROP2 Fab LC | DRFSGSGSGTDFTLTISSLQPEDFAVYYCQ | |
| Vh: SP34.185 scFv + TROP2 | QHYITPLTFGAGTKVEIKRTVAAPSVFIFPP | |
| Fab HC (R98A) HisTag | SDEQLKSGTASVVCLLNNFYPREAKVQW | |
| | KVDNALQSGNSQESVTEQDSKDSTYSLSS | |
| | TLTLSKADYEKHKVYACEVTHQGLSSPVT | |
| | KSFNRGEC | |
| TCE-6 HC | EVQLVESGGGLVQPGGSLKLSCAASGFTF | 94 |
| TROP2 Fab (HC R98A) TCE | NKYAMNWVRQAPGKGLEWVARIRSKYN | |
| V1: TROP2 Fab LC | NYATYYADSVKDRFTISRDDSKNTAYLQ | |
| Vh: SP34.185 scFv + TROP2 | MNNLKTEDTAVYYCVRHGNFGNSYISYW | |
| Fab HC (R98A) HisTag | AYWGQGTLVTVSSGGGGSGGGGSGGGGS | |
| | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSQ | |
| | VQLQQSGSELKKPGASVKVSCKASGYTFT | |
| | NYGMNWVKQAPGQGLKWMGWINTYTG | |
| | EPTYTDDFKGRFAFSLDTSVSTAYLQISSL | |
| | KADDTAVYFCAAGGFGSSYWYFDVWGQ | |
| | GSLVTVSSASTKGPSVFPLAPSSKSTSGGT | |
| | AALGCLVKDYFPEPVTVSWNSGALTSGVH | |
| | TFPAVLQSSGLYSLSSVVTVPSSSLGTQTYI | |
| | CNVNHKPSNTKVDKKVEPKSC | |
| TCE-7 LC | DIQLTQSPSSLSASVGDRVSITCKASQDVSI | 95 |
| TROP2 Fab (HC F108A) TCE | AVAWYQQKPGKAPKLLIYSASYRYTGVP | |
| V1: TROP2 Fab LC | DRFSGSGSGTDFTLTISSLQPEDFAVYYCQ | |
| Vh: SP34.185 scFv + TROP2 | QHYITPLTFGAGTKVEIKRTVAAPSVFIFPP | |
| Fab HC (F108A) HisTag | SDEQLKSGTASVVCLLNNFYPREAKVQW | |
| | KVDNALQSGNSQESVTEQDSKDSTYSLSS | |
| | TLTLSKADYEKHKVYACEVTHQGLSSPVT | |
| | KSFNRGEC | |
| TCE-7 HC | EVQLVESGGGLVQPGGSLKLSCAASGFTF | 96 |
| TROP2 Fab (HC F108A) TCE | NKYAMNWVRQAPGKGLEWVARIRSKYN | |
| V1: TROP2 Fab LC | NYATYYADSVKDRFTISRDDSKNTAYLQ | |
| Vh: SP34.185 scFv + TROP2 | MNNLKTEDTAVYYCVRHGNFGNSYISYW | |
| Fab HC (F108A) HisTag | AYWGQGTLVTVSSGGGGSGGGGSGGGGS | |
| | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSQ | |
| | VQLQQSGSELKKPGASVKVSCKASGYTFT | |
| | NYGMNWVKQAPGQGLKWMGWINTYTG | |
| | EPTYTDDFKGRFAFSLDTSVSTAYLQISSL | |
| | KADDTAVYFCARGGFGSSYWYADVWGQ | |
| | GSLVTVSSASTKGPSVFPLAPSSKSTSGGT | |
| | AALGCLVKDYFPEPVTVSWNSGALTSGVH | |
| | TFPAVLQSSGLYSLSSVVTVPSSSLGTQTYI | |
| | CNVNHKPSNTKVDKKVEPKSC | |
| TCE-8 LC | DIQLTQSPSSLSASVGDRVSITCKASQDVSI | 97 |
| TROP2 Fab (HC D109A) TCE | AVAWYQQKPGKAPKLLIYSASYRYTGVP | |
| V1: TROP2 Fab LC | DRFSGSGSGTDFTLTISSLQPEDFAVYYCQ | |
| Vh: SP34.185 scFv + TROP2 | QHYITPLTFGAGTKVEIKRTVAAPSVFIFPP | |
| Fab HC (D109A) HisTag | SDEQLKSGTASVVCLLNNFYPREAKVQW | |
| | KVDNALQSGNSQESVTEQDSKDSTYSLSS | |
| | TLTLSKADYEKHKVYACEVTHQGLSSPVT | |
| | KSFNRGEC | |
| TCE-8 HC | EVQLVESGGGLVQPGGSLKLSCAASGFTF | 98 |
| TROP2 Fab (HC D109A) TCE | NKYAMNWVRQAPGKGLEWVARIRSKYN | |
| V1: TROP2 Fab LC | NYATYYADSVKDRFTISRDDSKNTAYLQ | |
| Vh: SP34.185 scFv + TROP2 | MNNLKTEDTAVYYCVRHGNFGNSYISYW | |
| Fab HC (D109A) HisTag | AYWGQGTLVTVSSGGGGSGGGGSGGGGS | |
| | QTVVTQEPSLTVSPGGTVTLTCGSSTGAVT | |
| | SGNYPNWVQQKPGQAPRGLIGGTKFLAPG | |
| | TPARFSGSLLGGKAALTLSGVQPEDEAEY | |
| | YCVLWYSNRWVFGGGTKLTVLGGGGSQ | |
| | VQLQQSGSELKKPGASVKVSCKASGYTFT | |
| | NYGMNWVKQAPGQGLKWMGWINTYTG | |
| | EPTYTDDFKGRFAFSLDTSVSTAYLQISSL | |
| | KADDTAVYFCARGGFGSSYWYFAVWGQ | |
| | GSLVTVSSASTKGPSVFPLAPSSKSTSGGT | |
| | AALGCLVKDYFPEPVTVSWNSGALTSGVH | |
| | TFPAVLQSSGLYSLSSVVTVPSSSLGTQTYI | |
| | CNVNHKPSNTKVDKKVEPKSC | |

TABLE 8
TROP2- T cell engager (TCE) sequences

| Construct | | |
|---|---|---|
| Description | Amino Acid Sequence (N to C) | SEQ ID NO: |
| TCE-9 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-9 HC | EVQLVESGGGLVQPGGSLKLSCAASGFTFNKYAMNWVRQAP | 159 |
| anti-CD3 scFv wt + | GKGLEWVARIRSKYNNYATYYADSVKDRFTISRDDSKNTAYL | |
| linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-10 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-10 HC | EVQLVESGGGLVQPGGSLKLSCAASGFTFNKYAMNWVRQAP | 160 |
| anti-CD3 scFv | GKGLEWVARIRSKYNNYATYYADSVKDRFTISRDDSKNTAYL | |
| (V99A) + linker + | QMNNLKTEDTAVYYCARHGNFGNSYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-11 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-11 HC | EVQLVESGGGLVQPGGSLKLSCAASGFTFNKYAMNWVRQAP | 161 |
| anti-CD3 scFv | GKGLEWVARIRSKYNNYATYYADSVKDRFTISRDDSKNTAYL | |
| (R100A) + linker + | QMNNLKTEDTAVYYCVAHGNFGNSYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-12 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-12 HC | EVQLVESGGGLVQPGGSLKLSCAASGFTFNKYAMNWVRQAP | 162 |
| anti-CD3 scFv | GKGLEWVARIRSKYNNYATYYADSVKDRFTISRDDSKNTAYL | |
| (H101A) + linker + | QMNNLKTEDTAVYYCVRAGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-13 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-13 HC | EVQLVESGGGLVQPGGSLKLSCAASGFTFNKYAMNWVRQAP | 163 |
| anti-CD3 scFV | GKGLEWVARIRSKYNNYATYYADSVKDRFTISRDDSKNTAYL | |
| (G102A) + linker + | QMNNLKTEDTAVYYCVRHANFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-14 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-14 HC | EVQLVESGGGLVQPGGSLKLSCAASGFTFNKYAMNWVRQAP | 164 |
| anti-CD3 scFv | GKGLEWVARIRSKYNNYATYYADSVKDRFTISRDDSKNTAYL | |
| (N103A) + linker + | QMNNLKTEDTAVYYCVRHGAFGNSYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-15 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-15 HC | EVQLVESGGGLVQPGGSLKLSCAASGFTFNKYAMNWVRQAP | 165 |
| anti-CD3 scFv | GKGLEWVARIRSKYNNYATYYADSVKDRFTISRDDSKNTAYL | |
| (F104A) + linker + | QMNNLKTEDTAVYYCVRHGNAGNSYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-16 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-16 HC | EVQLVESGGGLVQPGGSLKLSCAASGFTFNKYAMNWVRQAP | 166 |
| anti-CD3 scFv | GKGLEWVARIRSKYNNYATYYADSVKDRFTISRDDSKNTAYL | |
| (G105A) + linker + | QMNNLKTEDTAVYYCVRHGNFANSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-17 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-17 HC | EVQLVESGGGLVQPGGSLKLSCAASGFTFNKYAMNWVRQAP | 167 |
| anti-CD3 scFv | GKGLEWVARIRSKYNNYATYYADSVKDRFTISRDDSKNTAYL | |
| (N106A) + linker + | QMNNLKTEDTAVYYCVRHGNFGASYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-18 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-18 HC | EVQLVESGGGLVQPGGSLKLSCAASGFTFNKYAMNWVRQAP | 168 |
| anti-CD3 scFv | GKGLEWVARIRSKYNNYATYYADSVKDRFTISRDDSKNTAYL | |
| (S107A) + linker + | QMNNLKTEDTAVYYCVRHGNFGNAYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-19 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-19 HC | EVQLVESGGGLVQPGGSLKLSCAASGFTFNKYAMNWVRQAP | 169 |
| anti-CD3 scFv | GKGLEWVARIRSKYNNYATYYADSVKDRFTISRDDSKNTAYL | |
| (Y108A) + linker + | QMNNLKTEDTAVYYCVRHGNFGNSAISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-20 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-20 HC | EVQLVESGGGLVQPGGSLKLSCAASGFTFNKYAMNWVRQAP | 170 |
| anti-CD3 scFv | GKGLEWVARIRSKYNNYATYYADSVKDRFTISRDDSKNTAYL | |
| (I109A) + linker + | QMNNLKTEDTAVYYCVRHGNFGNSYASYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-21 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-21 HC | EVQLVESGGGLVQPGGSLKLSCAASGFTFNKYAMNWVRQAP | 171 |
| anti-CD3 scFv | GKGLEWVARIRSKYNNYATYYADSVKDRFTISRDDSKNTAYL | |
| (S110A) + linker + | QMNNLKTEDTAVYYCVRHGNFGNSYIAYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-22 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-22 HC | EVQLVESGGGLVQPGGSLKLSCAASGFTFNKYAMNWVRQAP | 172 |
| anti-CD3 scFv | GKGLEWVARIRSKYNNYATYYADSVKDRFTISRDDSKNTAYL | |
| (Y111A) + linker + | QMNNLKTEDTAVYYCVRHGNFGNSYISAWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-23 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-23 HC | EVQLVESGGGLVQPGGSLKLSCAASGFTFNKYAMNWVRQAP | 173 |
| anti-CD3 scFv | GKGLEWVARIRSKYNNYATYYADSVKDRFTISRDDSKNTAYL | |
| (W112A) + linker + | QMNNLKTEDTAVYYCVRHGNFGNSYISYAAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-24 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-24 HC | EVQLVESGGGLVQPGGSLKLSCAASGFTFNKYAMNWVRQAP | 174 |
| anti-CD3 scFv | GKGLEWVARIRSKYNNYATYYADSVKDRFTISRDDSKNTAYL | |
| (Y114A) + linker + | QMNNLKTEDTAVYYCVRHGNFGNSYISYWAAWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-25 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-25 HC | EVQLVESGGGLVQPGGSLKLSCAASGFTFNKYAMNWVRQAP | 175 |
| anti-CD3 scFv | GKGLEWVARIRSKYNNYATYYADSVKDRFTISRDDSKNTAYL | |
| (V231A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCALWYSNRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-26 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-26 HC | EVQLVESGGGLVQPGGSLKLSCAASGFTFNKYAMNWVRQAP | 176 |
| anti-CD3 scFv | GKGLEWVARIRSKYNNYATYYADSVKDRFTISRDDSKNTAYL | |
| (L232A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVAWYSNRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-27 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-27 HC | EVQLVESGGGLVQPGGSLKLSCAASGFTFNKYAMNWVRQAP | 177 |
| anti-CD3 scFv | GKGLEWVARIRSKYNNYATYYADSVKDRFTISRDDSKNTAYL | |
| (W233A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLAYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-28 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-28 HC | EVQLVESGGGLVQPGGSLKLSCAASGFTFNKYAMNWVRQAP | 178 |
| anti-CD3 scFv | GKGLEWVARIRSKYNNYATYYADSVKDRFTISRDDSKNTAYL | |
| (Y234A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWASNRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-29 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-29 HC | EVQLVESGGGLVQPGGSLKLSCAASGFTFNKYAMNWVRQAP | 179 |
| anti-CD3 scFv | GKGLEWVARIRSKYNNYATYYADSVKDRFTISRDDSKNTAYL | |
| (S235A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYANRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-30 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-30 HC | EVQLVESGGGLVQPGGSLKLSCAASGFTFNKYAMNWVRQAP | 180 |
| anti-CD3 scFv | GKGLEWVARIRSKYNNYATYYADSVKDRFTISRDDSKNTAYL | |
| (N236A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSARWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-31 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-31 HC | EVQLVESGGGLVQPGGSLKLSCAASGFTFNKYAMNWVRQAP | 181 |
| anti-CD3 scFv | GKGLEWVARIRSKYNNYATYYADSVKDRFTISRDDSKNTAYL | |
| (R237A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNAWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-32 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-32 HC | EVQLVESGGGLVQPGGSLKLSCAASGFTFNKYAMNWVRQAP | 182 |
| anti-CD3 scFv | GKGLEWVARIRSKYNNYATYYADSVKDRFTISRDDSKNTAYL | |
| (W238A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRAVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-33 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-33 HC | EVQLVESGGGLVQPGGSLKLSCAASGFTFNKYAMNWVRQAP | 183 |
| anti-CD3 scFv | GKGLEWVARIRSKYNNYATYYADSVKDRFTISRDDSKNTAYL | |
| (V239A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWAFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-34 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-34 HC | EVQLVESGGGLVQPGGSLKLSCAASGFTFNKYAMNWVRQAP | 184 |
| anti-CD3 scFv | GKGLEWVARIRSKYNNYATYYADSVKDRFTISRDDSKNTAYL | |
| (F240A) + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-35 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-35 HC | EVQLVESGGGLVQPGGSLKLSCAASGFTFNKYAMNWVRQAS | 185 |
| anti-CD3 scFv + | GKGLEWVGRIRSKYNNYATYYADSVKDRFTISRDDSKNTAYL | |
| linker + anti- | QMNSLKTEDTAVYYCVRHGNFGNSYISYWAYWGQGTLVTV | |
| TROP2 HC wt | SSGGGGSGGGGSGGGGSQTVVTQEPSFSVSPGGTVTLTCRSST | |
| | GAVTSGNYANWVQQTPGQAPRGLIGGTKFLAPGVPDRFSGSI | |
| | LGNKAALTITGAQADDESDYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-36 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-36 HC | EVQLVESGGGLVQPGGSLKLSCAASGFTFQKYAMNWVRQAP | 186 |
| anti-CD3 scFv + | GKGLEWVARIRSKYNNYATYYADSVKDRFTISRDDSKNTAYL | |
| linker + anti- | QMNNLKTEDTAVYYCVRHGNFGNSYVSYWAYWGQGTLVT | |
| TROP2 HC wt | VSSGGGGSGGGGSGGGGSQTVVTQEPSLTVSPGGTVTLTCGS | |
| | STGAVTSANYPNWVQQKPGQAPRGLIGGTKFLAPGTPARFSG | |
| | SLLGGKAALTLSGVQPEDEAEYYCALWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-37 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-37 HC | EVQLVESGGGLVQPGGSLKLSCAASGFTFNKYAMNWVRQAP | 187 |
| anti-CD3 scFv wt + | GKGLEWVARIRSKYNNYATYYADSVKDRFTISRDDSKNTAYL | |
| linker + anti- | QMNNLKTEDTAVYYCVRHGNFGNSYISYWAYWGQGTLVTV | |
| TROP2 (F108A) | SSGGGGSGGGGSGGGGSQTVVTQEPSLTVSPGGTVTLTCGSST | |
| HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-38 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-38 HC | EVQLVESGGGLVQPGGSLKLSCAASGFTFNKYAMNWVRQAP | 188 |
| anti-CD3 scFv | GKGLEWVARIRSKYNNYATYYADSVKDRFTISRDDSKNTAYL | |
| (H101A) + linker + | QMNNLKTEDTAVYYCVRAGNFGNSYISYWAYWGQGTLVTV | |
| anti-TROP2 | SSGGGGSGGGGSGGGGSQTVVTQEPSLTVSPGGTVTLTCGSST | |
| (F108A) HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-39 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-39 HC | EVQLVESGGGLVQPGGSLKLSCAASGFTFNKYAMNWVRQAP | 189 |
| anti-CD3 scFv | GKGLEWVARIRSKYNNYATYYADSVKDRFTISRDDSKNTAYL | |
| (F104A) + linker + | QMNNLKTEDTAVYYCVRHGNAGNSYISYWAYWGQGTLVT | |
| anti-TROP2 | VSSGGGGSGGGGSGGGGSQTVVTQEPSLTVSPGGTVTLTCGS | |
| (F108A) HC | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYADVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-40 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-40 HC | EVQLVESGGGLVQPGGSLKLSCAASGFTFNKYAMNWVRQAP | 190 |
| anti-CD3 scFv | GKGLEWVARIRSKYNNYATYYADSVKDRFTISRDDSKNTAYL | |
| (F240A) + linker + | QMNNLKTEDTAVYYCVRHGNFGNSYISYWAYWGQGTLVTV | |
| anti-TROP2 | SSGGGGSGGGGSGGGGSQTVVTQEPSLTVSPGGTVTLTCGSST | |
| (F108A) HC | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYADVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-41 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-41 HC | EVQLVESGGGLVQPGGSLKLSCAASGFTFNKYAMNWVRQAS | 191 |
| anti-CD3 scFv + | GKGLEWVGRIRSKYNNYATYYADSVKDRFTISRDDSKNTAYL | |
| linker + anti- | QMNSLKTEDTAVYYCVRHGNFGNSYISYWAYWGQGTLVTV | |
| TROP2 (F108A) | SSGGGGSGGGGSGGGGSQTVVTQEPSFSVSPGGTVTLTCRSST | |
| HC | GAVTSGNYANWVQQTPGQAPRGLIGGTKFLAPGVPDRFSGSI | |
| | LGNKAALTITGAQADDESDYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-42 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-42 HC | EVQLVESGGGLVQPGGSLKLSCAASGFTFQKYAMNWVRQAP | 192 |
| anti-CD3 scFv + | GKGLEWVARIRSKYNNYATYYADSVKDRFTISRDDSKNTAYL | |
| linker + anti- | QMNNLKTEDTAVYYCVRHGNFGNSYVSYWAYWGQGTLVT | |
| TROP2 (F108A) | VSSGGGGSGGGGSGGGGSQTVVTQEPSLTVSPGGTVTLTCGS | |
| HC | STGAVTSANYPNWVQQKPGQAPRGLIGGTKFLAPGTPARFSG | |
| | SLLGGKAALTLSGVQPEDEAEYYCALWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYADVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-43 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-43 HC | EVQLVESGGGLVQPGGSLKLSCAASGFTFNKYAMNWVRQAP | 193 |
| anti-CD3 scFv wt + | GKGLEWVARIRSKYNNYATYYADSVKDRFTISRDDSKNTAYL | |
| linker + anti- | QMNNLKTEDTAVYYCVRHGNFGNSYISYWAYWGQGTLVTV | |
| TROP2 (D109A) | SSGGGGSGGGGSGGGGSQTVVTQEPSLTVSPGGTVTLTCGSST | |
| HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFAVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-44 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-44 HC | EVQLVESGGGLVQPGGSLKLSCAASGFTFNKYAMNWVRQAP | 194 |
| anti-CD3 scFv | GKGLEWVARIRSKYNNYATYYADSVKDRFTISRDDSKNTAYL | |
| (H101A) + linker + | QMNNLKTEDTAVYYCVRAGNFGNSYISYWAYWGQGTLVTV | |
| anti-TROP2 | SSGGGGSGGGGSGGGGSQTVVTQEPSLTVSPGGTVTLTCGSST | |
| (D109A) HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFAVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-45 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-45 HC | EVQLVESGGGLVQPGGSLKLSCAASGFTFNKYAMNWVRQAP | 195 |
| anti-CD3 scFv | GKGLEWVARIRSKYNNYATYYADSVKDRFTISRDDSKNTAYL | |
| (F104A) + linker + | QMNNLKTEDTAVYYCVRHGNAGNSYISYWAYWGQGTLVT | |
| anti-TROP2 | VSSGGGGSGGGGSGGGGSQTVVTQEPSLTVSPGGTVTLTCGS | |
| (D109A) HC | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFAVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-46 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-46 HC | EVQLVESGGGLVQPGGSLKLSCAASGFTFNKYAMNWVRQAP | 196 |
| anti-CD3 scFv | GKGLEWVARIRSKYNNYATYYADSVKDRFTISRDDSKNTAYL | |
| (F240A) + linker + | QMNNLKTEDTAVYYCVRHGNFGNSYISYWAYWGQGTLVTV | |
| anti-TROP2 | SSGGGGSGGGGSGGGGSQTVVTQEPSLTVSPGGTVTLTCGSST | |
| (D109A) HC | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFAVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-47 LC | DIQLTQSPSSLSASVGDRVSITCKASQDVSIAVAWYQQKPGKA | 158 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-47 HC | EVQLVESGGGLVQPGGSLKLSCAASGFTFQKYAMNWVRQAP | 197 |
| anti-CD3 scFv + | GKGLEWVARIRSKYNNYATYYADSVKDRFTISRDDSKNTAYL | |
| linker + anti- | QMNNLKTEDTAVYYCVRHGNFGNSYVSYWAYWGQGTLVT | |
| TROP2 (D109A) | VSSGGGGSGGGGSGGGGSQTVVTQEPSLTVSPGGTVTLTCGS | |
| HC | STGAVTSANYPNWVQQKPGQAPRGLIGGTKFLAPGTPARFSG | |
| | SLLGGKAALTLSGVQPEDEAEYYCALWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFAVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |

### Polynucleotides Encoding Recombinant Antibodies or Antigen Binding Fragment Compositions

Disclosed herein, in some embodiments, are isolated recombinant nucleic acid molecules encoding a recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2, and wherein CDR3-L comprises an amino acid sequence of X₁X₂HYX₃X₄X₅X₆X₇, wherein X₁ is Q, S, T, D, N, E, H, R, K, or A, X₂ is Q, S, T, D, N, E, H, K, R, or A, X₃ is I, G, P, V, L, M, S, T, or A, X₄ is T, G, S, M, H, N, Q, or A, X₅ is P, G, V, L, I, M, S, T, or A, X₆ is L, G, P, V, I, M, S, T, or A, and X₇ is T, G, S, M, H, N, Q, or A, wherein CDR1-H comprises the amino acid sequence of SEQ ID NO: 13, wherein CDR2-H comprises the amino acid sequence of SEQ ID NO: 14, and wherein CDR3-H comprises an amino acid sequence of AX₈X₉GX₁₀X₁₁X₁₂X₁₃YWX₁₄X₁₅ X₁₆X₁₇, wherein X₈ is R, S, T, Q, D, E, N, H, K, or A, X₉ is G, P, V, L, I, M, S, T, or A, X₁₀ is F, Y, W, V, L, I, G, or A, X₁₁ is G, P, V, L, I, M, S, T, or A, X₁₂ is S, G, T, M, N, Q, H, or A, X₁₃ is S, G, T, M, N, Q, H, or A, X₁₄ is Y, F, W, V, L, I, G, or A, X₁₅ is F, Y, W, V, L, I, G, or A, X₁₆ is D, Q, N, E, S, T, H, K, R, or A, and X₁₇ is V, G, P, L, I, M, S, T, or A, and wherein when CDR3-H comprises the amino acid sequence of SEQ ID NO: 15, then CDR3-L does not comprise the amino acid sequence of SEQ ID NO: 3. In some embodiments, X₁ is Q, S, T, D, N, E, or A; X₂ is Q, S, T, D, N, E, or A; X₃ is I, G, P, V, L, M, or A; X₄ is T, G, S, M, H, N, Q, or A; X₅ is P, G, V, L, I, M, or A; X₆ is L, G, P, V, I, M, or A; X₇ is T, G, S, M, H, N, Q, or A; X₈ is R, H, K, or A; X₉ is G, P, V, L, I, M, S, T, or A; X₁₀ is F, Y, W, V, L, I, or A; X₁₁ is G, P, V, L, I, M, S, T, or A; X₁₂ is S, G, T, M, N, Q, or A; X₁₃ is S, G, T, M, N, Q, or A; X₁₄ is Y, F, W, V, L, I, or A; X₁₅ is F, Y, W, V, L, I, or A; X₁₆ is D, Q, N, E, S, T, or A; and X₁₇ is V, G, P, L, I, M, or A.

Further disclosed herein, in some embodiments, are isolated recombinant nucleic acid molecules encoding a recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 6, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 7, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 18; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14, CDR3-H: SEQ ID NO:21; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14; CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 23; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 24; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28

### Pharmaceutical Compositions

Disclosed herein, in some embodiments, are pharmaceutical compositions comprising: (a) the recombinant antibodies or antigen binding fragments thereof described herein and (b) a pharmaceutically acceptable excipient. In some embodiments, the recombinant antibody or antigen binding fragment thereof comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2, and wherein CDR3-L comprises an amino acid sequence of X₁X₂HYX₃X₄X₅X₆X₇, wherein X₁ is Q, S, T, D, N, E, H, R, K, or A, X₂ is Q, S, T, D, N, E, H, K, R, or A, X₃ is I, G, P, V, L, M, S, T, or A, X₄ is T, G, S, M, H, N, Q, or A, X₅ is P, G, V, L, I, M, S, T, or A, X₆ is L, G, P, V, I, M, S, T, or A, and X₇ is T, G, S, M, H, N, Q, or A, wherein CDR1-H comprises the amino acid sequence of SEQ ID NO: 13, wherein CDR2-H comprises the amino acid sequence of SEQ ID NO: 14, and wherein CDR3-H comprises an amino acid sequence of AX₈X₉GX₁₀X₁₁X₁₂X₁₃YWX₁₄X₁₅ X₁₆X₁₇, wherein X₈ is R, S, T, Q, D, E, N, H, K, or A, X₉ is G, P, V, L, I, M, S, T, or A, X₁₀ is F, Y, W, V, L, I, G, or A, Xu is G, P, V, L, I, M, S, T, or A, X₁₂ is S, G, T, M, N, Q, H, or A, X₁₃ is S, G, T, M, N, Q, H, or A, X₁₄ is Y, F, W, V, L, I, G, or A, X₁₅ is F, Y, W, V, L, I, G, or A, X₁₆ is D, Q, N, E, S, T, H, K, R, or A, and X₁₇ is V, G, P, L, I, M, S, T, or A, and wherein when CDR3-H comprises the amino acid sequence of SEQ ID NO: 15, then CDR3-L does not comprise the amino acid sequence of SEQ ID NO: 3. In some embodiments, X₁ is Q, S, T, D, N, E, or A; X₂ is Q, S, T, D, N, E, or A; X₃ is I, G, P, V, L, M, or A; X₄ is T, G, S, M, H, N, Q, or A; X₅ is P, G, V, L, I, M, or A; X₆ is L, G, P, V, I, M, or A; X₇ is T, G, S, M, H, N, Q, or A; X₈ is R, H, K, or A; X₉ is G, P, V, L, I, M, S, T, or A; X₁₀ is F, Y, W, V, L, I, or A; X₁₁ is G, P, V, L, I, M, S, T, or A; X₁₂ is S, G, T, M, N, Q, or A; X₁₃ is S, G, T, M, N, Q, or A; X₄ is Y, F, W, V, L, I, or A; X₁₅ is F, Y, W, V, L, I, or A; X₁₆ is D, Q, N, E, S, T, or A; and X₁₇ is V, G, P, L, I, M, or A.

In some embodiments, the recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 6, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 7, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 18; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14, CDR3-H: SEQ ID NO:21; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14; CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 23; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 24; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

For administration to a subject, the recombinant antibodies or antigen binding fragments thereof as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Treatment

In some embodiments, are methods of treating cancer in a subject need in need thereof comprising administering to the subject a recombinant antibody or antigen binding fragment thereof as described herein. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer in a subject need in need thereof comprising administering to the subject a polypeptide or polypeptide complex as described herein. In some embodiments, are methods of treating breast cancer, lung cancer, urothelial cancer, endometrial cancer, ovarian cancer, prostate cancer, pancreatic cancer, gastric cancer, colon cancer, head and neck cancer, and glioma in a subject need in need thereof comprising administering to the subject a polypeptide or polypeptide complex as described herein.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Machl™, DH10B™ TOP10, DH5a, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx^{T}M Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to TROP2 as defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes

### EMBODIMENTS

Embodiment 1 comprises a recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1; wherein CDR2-L comprises the amino acid sequence of SEQ ID NO: 2; and wherein CDR3-L comprises an amino acid sequence of X₁X₂HYX₃X₄X₅X₆X₇; wherein X₁ is Q, S, T, D, N, E, H, K, R, or A; X₂ is Q, S, T, D, N, E, H, K, R, or A; X₃ is I, G, P, V, L, M, S, T, or A; X₄ is T, G, S, M, H, N, Q, or A; X₅ is P, G, V, L, I, M, S, T, or A; X₆ is L, G, P, V, I, M, S, T, or A; and X₇ is T, G, S, M, H, N, Q, or A; wherein CDR1-H comprises the amino acid sequence of SEQ ID NO: 13; wherein CDR2-H comprises the amino acid sequence of SEQ ID NO: 14; and wherein CDR3-H comprises an amino acid sequence of AX₈X₉GX₁₀X₁₁X₁₂X₁₃YWX₁₄X₁₅X₁₆X₁₇; wherein X₈ is R, S, T, Q, D, E, H, K, N, H, K, or A; X₉ is G, P, V, L, I, M, S, T, or A; X₁₀ is F, Y, W, V, L, I, G, or A; Xu is G, P, V, L, I, M, S, T, or A; X₁₂ is S, G, T, M, N, Q, H, or A; X₁₃ is S, G, T, M, N, Q, H, or A; X₁₄ is Y, F, W, V, L, I, G, or A; X₁₅ is F, Y, W, V, L, I, G, or A; X₁₆ is D, Q, N, E, S, T, H, K, R, or A; and X₁₇ is V, G, P, L, I, M, S, T, or A; wherein when CDR3-H comprises the amino acid sequence of SEQ ID NO: 15, then CDR3-L does not comprise the amino acid sequence of SEQ ID NO: 3.

Embodiment 2 comprises the recombinant antibody or antigen binding fragment thereof of embodiment 1, wherein X₁ is Q, N, D, E, or A; X₂ is Q, N, D, E, or A; X₃ is I, V, L, or A; X₄ is T, S, or A; X₅ is P, G, or A; X₆ is L, V, I, or A; X₇ is T, S, or A; X₈ is R, K, or A; X₉ is G, S, T, or A; X₁₀ is F, Y, or A; X₁₁ is G, S, T, or A; X₁₂ is S, G, T, or A; X₁₃ is S, G, T, or A; X₁₄ is Y, W, F, or A; X₁₅ is F, Y, W, or A; X₁₆ is D, E, Q, N, or A; and X₁₇ is V, L, I, or A.

Embodiment 3 comprises the recombinant antibody or antigen binding fragment thereof of embodiment 1, wherein X₁ is Q; and X₆ is L.

Embodiment 4 comprises the recombinant antibody or antigen binding fragment thereof of embodiment 3, wherein X₈ is R; X₁₀ is F; X₁₁ is G; X₁₄ is Y; X₁₅ is F; and X₁₆ is D.

Embodiment 5 comprises the recombinant antibody or antigen binding fragment thereof of embodiment 1, wherein X₁ is Q, S, T, D, N, E, or A; X₂ is Q, S, T, D, N, E, or A; X₃ is I, G, P, V, L, M, or A; X₄ is T, G, S, M, H, N, Q, or A; X₅ is P, G, V, L, I, M, or A; X₆ is L, G, P, V, I, M, or A; X₇ is T, G, S, M, H, N, Q, or A; X₈ is R, H, K, or A; X₉ is G, P, V, L, I, M, S, T, or A; X₁₀ is F, Y, W, V, L, I, or A; X₁ is G, P, V, L, I, M, S, T, or A; X₁₂ is S, G, T, M, N, Q, or A; X₁₃ is S, G, T, M, N, Q, or A; X₁₄ is Y, F, W, V, L, I, or A; X₁₅ is F, Y, W, V, L, I, or A; X₁₆ is D, Q, N, E, S, T, or A; and X₁₇ is V, G, P, L, I, M, or A.

Embodiment 6 comprises the recombinant antibody or antigen binding fragment thereof of embodiment 5, wherein X₁ is Q, N, or A; X₂ is Q, N, or A; X₃ is I, V, L, or A; X₄ is T, S, or A; X₅ is P, G, or A; X₆ is L, V, I, or A; X₇ is T, S, or A; X₈ is R, K, or A; X₉ is G, V, S, T, or A; X₁₀ is F, Y, or A; X₁₁ is G, V, S, T, or A; X₁₂ is S, G, T, or A; X₁₃ is S, G, T, or A; X₁₄ is Y, W, or A; X₁₅ is F, Y, or A; X₁₆ is D, E, or A; and X₁₇ is V, G, L, I, or A.

Embodiment 7 comprises the recombinant antibody or antigen binding fragment thereof of embodiment 5, wherein X₁ is Q; and X₆ is L.

Embodiment 8 comprises the recombinant antibody or antigen binding fragment thereof of embodiment 7, wherein X₈ is R; X₁₀ is F; X₁₁ is G; X₁₄ is Y; X₁₅ is F; and X₁₆ is D.

Embodiment 9 comprises the recombinant antibody or antigen binding fragment thereof of embodiment 1, wherein CDR3-L comprises an amino acid selected from SEQ ID NOs: 3-5 and 8-12.

Embodiment 10 comprises the recombinant antibody or antigen binding fragment thereof of embodiments 1 or 9, wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11, and CDR1-H: SEQ ID NO: 13, CDR2-H: SEQ ID NO: 14, CDR3-H: SEQ ID NO: 15; and CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15.

Embodiment 11 comprises the recombinant antibody or antigen binding fragment thereof of any one of embodiments 1 and 9-10, wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; and CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15.

Embodiment 12 comprises the recombinant antibody or antigen binding fragment thereof of embodiment 1, wherein CDR3-H comprises an amino acid selected from SEQ ID NOs: 16-17, 19-22, and 25-28.

Embodiment 13 comprises the recombinant antibody or antigen binding fragment thereof of embodiments 1 or 2, wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14; CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

Embodiment 14 comprises the recombinant antibody or antigen binding fragment thereof of any one of embodiments 1 and 12-13, wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 21; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 22; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

Embodiment 15 comprises a recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the immunoglobulin light chain and the immunoglobulin heavy chain comprise a set of CDR sequences selected from the group consisting of: CDR1-L: SEQ ID NO: 1, CDR2-L: SEQ ID NO: 2, CDR3-L: SEQ ID NO: 4, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 5, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 6, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 7, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 8, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 9, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 10, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 11, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 12, and CDR1-H: SEQ ID NO: 13, CDR2-H: SEQ ID NO: 14, CDR3-H: SEQ ID NO: 15; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 16; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 17; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 18; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 19; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 20; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14, CDR3-H: SEQ ID NO: 21; CDR1-L: SEQ ID NO: 1, CDR2-L: SEQ ID NO: 2; CDR3-L: SEQ ID NO: 3, and CDR1-H: SEQ ID NO: 13, CDR2-H: SEQ ID NO: 14; CDR3-H: SEQ ID NO: 22; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 23; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 24; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 25; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 26; CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 27; and CDR1-L: SEQ ID NO: 1, CDR2-L: SEQ ID NO: 2, CDR3-L: SEQ ID NO: 3, and CDR1-H: SEQ ID NO: 13, CDR2-H: SEQ ID NO: 14, CDR3-H: SEQ ID NO: 28.

Embodiment 16 comprises the recombinant antibody or antigen binding fragment thereof of any one of embodiments 1-15, wherein the TROP2 binding domain comprises a Fab, Fab′, (Fab′)₂ or a single chain variable fragment (scFv).

Embodiment 17 comprises the recombinant antibody or antigen binding fragment thereof of any one of embodiments 1-16, wherein the TROP2 binding domain is a Fab.

Embodiment 18 comprises the recombinant antibody or antigen binding fragment thereof of any one of embodiments 1-17, wherein the immunoglobulin light chain comprises a variable domain of an immunoglobulin kappa (IgK) or immunoglobulin lambda (IgL) light chain.

Embodiment 19 comprises the recombinant antibody or antigen binding fragment thereof of any one of embodiments 1-18, wherein the immunoglobulin heavy chain comprises a variable domain of an IgG1, IgG2, IgG3, or IgG4 heavy chain.

Embodiment 20 comprises the recombinant antibody or antigen binding fragment thereof of any one of embodiments 1-19, wherein the immunoglobulin light chain comprises an amino acid sequence with at least 90%, 95%, 96%, 97%, 98%, 99%, or 100% identity to any one of SEQ ID NOs: 31, 33, 35, 37, 39, 41, 43, 45, 47, 49, 51, 53, 55, 57, 59, 61, 63, 65, 67, 69, 71, or 73.

Embodiment 21 comprises the recombinant antibody or antigen binding fragment thereof of any one of embodiments 1-20, wherein the immunoglobulin heavy chain comprises an amino acid sequence with at least 90%, 95%, 96%, 97%, 98%, 99%, or 100% identity to any one of SEQ ID NOs: 32, 34, 36, 38, 40, 42, 44, 46, 48, 50, 52, 54, 56, 58, 60, 62, 64, 66, 68, 70, 72, or 74.

Embodiment 22 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 31 and the immunoglobulin heavy chain comprises an amino acid sequence with at least 90%, 95%, 96%, 97%, 98%, 99%, or 100% identity to SEQ ID NO: 32.

Embodiment 23 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 33 and the immunoglobulin heavy chain comprises an amino acid sequence with at least 90%, 95%, 96%, 97%, 98%, 99%, or 100% identity to SEQ ID NO: 34.

Embodiment 24 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 35 and the immunoglobulin heavy chain comprises an amino acid sequence with at least 90%, 95%, 96%, 97%, 98%, 99%, or 100% identity to SEQ ID NO: 36.

Embodiment 25 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 37 and the immunoglobulin heavy chain comprises an amino acid sequence with at least 90%, 95%, 96%, 97%, 98%, 99%, or 100% identity to SEQ ID NO: 38.

Embodiment 26 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 39 and the immunoglobulin heavy chain comprises an amino acid sequence with at least 90%, 95%, 96%, 97%, 98%, 99%, or 100% identity to SEQ ID NO: 40.

Embodiment 27 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 41 and the immunoglobulin heavy chain comprises an amino acid sequence with at least 90%, 95%, 96%, 97%, 98%, 99%, or 100% identity to SEQ ID NO: 42.

Embodiment 28 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 43 and the immunoglobulin heavy chain comprises an amino acid sequence with at least 90%, 95%, 96%, 97%, 98%, 99%, or 100% identity to SEQ ID NO: 44.

Embodiment 29 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 45 and the immunoglobulin heavy chain comprises an amino acid sequence with at least 90%, 95%, 96%, 97%, 98%, 99%, or 100% identity to SEQ ID NO: 46.

Embodiment 30 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 47 and the immunoglobulin heavy chain comprises an amino acid sequence with at least 90%, 95%, 96%, 97%, 98%, 99%, or 100% identity to SEQ ID NO: 48.

Embodiment 31 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 49 and the immunoglobulin heavy chain comprises an amino acid sequence with at least 90%, 95%, 96%, 97%, 98%, 99%, or 100% identity to SEQ ID NO: 50.

Embodiment 32 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 51 and the immunoglobulin heavy chain comprises an amino acid sequence with at least 90%, 95%, 96%, 97%, 98%, 99%, or 100% identity to SEQ ID NO: 52.

Embodiment 33 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 53 and the immunoglobulin heavy chain comprises an amino acid sequence with at least 90%, 95%, 96%, 97%, 98%, 99%, or 100% identity to SEQ ID NO: 54.

Embodiment 34 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 55 and the immunoglobulin heavy chain comprises an amino acid sequence with at least 90%, 95%, 96%, 97%, 98%, 99%, or 100% identity to SEQ ID NO: 56.

Embodiment 35 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 57 and the immunoglobulin heavy chain comprises an amino acid sequence with at least 90%, 95%, 96%, 97%, 98%, 99%, or 100% identity to SEQ ID NO: 58.

Embodiment 36 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 59 and the immunoglobulin heavy chain comprises an amino acid sequence with at least 90%, 95%, 96%, 97%, 98%, 99%, or 100% identity to SEQ ID NO: 60.

Embodiment 37 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 61 and the immunoglobulin heavy chain comprises an amino acid sequence with at least 90%, 95%, 96%, 97%, 98%, 99%, or 100% identity to SEQ ID NO: 62.

Embodiment 38 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 63 and the immunoglobulin heavy chain comprises an amino acid sequence with at least 90%, 95%, 96%, 97%, 98%, 99%, or 100% identity to SEQ ID NO: 64.

Embodiment 39 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 65 and the immunoglobulin heavy chain comprises an amino acid sequence with at least 90%, 95%, 96%, 97%, 98%, 99%, or 100% identity to SEQ ID NO: 66.

Embodiment 40 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 67 and the immunoglobulin heavy chain comprises an amino acid sequence with at least 90%, 95%, 96%, 97%, 98%, 99%, or 100% identity to SEQ ID NO: 68.

Embodiment 41 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 69 and the immunoglobulin heavy chain comprises an amino acid sequence with at least 90%, 95%, 96%, 97%, 98%, 99%, or 100% identity to SEQ ID NO: 70.

Embodiment 42 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 71 and the immunoglobulin heavy chain comprises an amino acid sequence with at least 90%, 95%, 96%, 97%, 98%, 99%, or 100% identity to SEQ ID NO: 72.

Embodiment 43 comprises the recombinant antibody or antigen binding fragment thereof of any one of embodiments 1-2 and 15-21, wherein the immunoglobulin light chain comprises an amino acid sequence with at least 90%, 95%, 96%, 97%, 98%, 99%, or 100% identity to SEQ ID NO: 73 and the immunoglobulin heavy chain comprises an amino acid sequence with at least 90%, 95%, 96%, 97%, 98%, 99%, or 100% identity to SEQ ID NO: 74.

Embodiment 44 comprises the recombinant antibody or antigen binding fragment thereof of any one of embodiments 1-2 and 15-21, wherein the TROP2 binding domain has weaker binding to TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions.

Embodiment 45 comprises the recombinant antibody or antigen binding fragment thereof of any one of embodiments 1-2 and 15-21, wherein the TROP2 binding domain has an increased EC₅₀ for TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions.

Embodiment 46 comprises the recombinant antibody or antigen binding fragment thereof of any one of embodiments 1-2 and 15-21, wherein the TROP2 binding domain has a faster off rate (larger k_{diss}) for TROP2 binding as compared to a TROP2 binding domain that comprises an immunoglobulin light chain according to SEQ ID NO: 29 and an immunoglobulin heavy chain according to SEQ ID NO: 30 as measured under substantially similar kinetic assay conditions.

Embodiment 47 comprises the recombinant antibody or antigen binding fragment thereof of any one of embodiments 1-46, wherein the recombinant antibody or antigen binding fragment further comprises a CD3 binding domain.

Embodiment 48 comprises the recombinant antibody or antigen binding fragment thereof of embodiment 47, wherein the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 75, wherein CDR2-L comprises the amino acid sequence of SEQ ID NO: 76, wherein CDR3-L comprises the amino acid sequence of SEQ ID NO: 77, wherein CDR1-H comprises the amino acid sequence of SEQ ID NO: 78, wherein CDR2-H comprises the amino acid sequence of SEQ ID NO: 79, and wherein CDR3-H comprises the amino acid sequence of SEQ ID NO: 80.

Embodiment 49 comprises the recombinant antibody or antigen binding fragment thereof of embodiment 47, wherein the CD3 binding domain comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 75; wherein CDR2-L comprises the amino acid sequence of SEQ ID NO: 76; wherein CDR3-L comprises the amino acid sequence of Z₁-Z₂-W-Z₃-Z₄-Z₅-Z₆-W-Z₇-Z₈; wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 78; wherein CDR2-H comprises the amino acid sequence of SEQ ID NO: 79; wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁; and wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

Embodiment 50 comprises the recombinant antibody or antigen binding fragment thereof of embodiment 49, wherein: Z₁ is V, G, L, I, or A; Z₂ is L, V, I, or A; Z₃ is Y, W, F, or A; Z₄ is S, G, T, or A; Z₅ is N, Q, D, E, or A; Z₆ is R, K, or A; Z₇ is V, G, L, I, or A; Z₈ is F, Y, W, or A; Z₉ is V, G, L, I, or A; Z₁₀ is R, K, or A; Z₁₂ is G, S, T, or A; Z₁₃ is F, Y, W, or A; Z₁₄ is G, S, T, or A; Z₁₅ is N, Q, D, E, or A; Z₁₆ is S, G, T, or A; Z₁₇ is Y, W, F, or A; Z₁₈ is I, V, L, or A; Z₁₉ is S, G, T, or A; Z₂₀ is W, Y, F, or A; and Z₂₁ is Y, W, F, or A.

Embodiment 51 comprises the recombinant antibody or antigen binding fragment thereof of embodiment 49 or 50, wherein: Z₈ is F.

Embodiment 52 comprises the recombinant antibody or antigen binding fragment thereof of any one of embodiments 49-51, wherein: Z₁₀ is R; Z₁₁ is H; Z₁₃ is F; Z₁₅ is I; Z₁₉ is S; and Z₂₀ is W.

Embodiment 53 comprises the recombinant antibody or antigen binding fragment thereof of embodiment 49, wherein CDR3-L comprises an amino acid sequence selected from SEQ ID NOs: 77, 105-106, 108-111, and 113-114.

Embodiment 54 comprises the recombinant antibody or antigen binding fragment thereof of embodiment 49, wherein the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; and CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80.

Embodiment 55 comprises the recombinant antibody or antigen binding fragment thereof of embodiment 49, wherein the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; and CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80.

Embodiment 56 comprises the antibody or antigen binding fragment thereof of embodiment 49, wherein CDR3-H comprises an amino acid sequence selected from SEQ ID NOs: 80, 116-119, 121-127, and 129-130.

Embodiment 57 comprises the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

Embodiment 58 comprises the recombinant antibody or antigen binding fragment thereof of embodiment 49, wherein the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

Embodiment 59 comprises the recombinant antibody or antigen binding fragment thereof of embodiment 47, wherein the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 107, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 112, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 120; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 128; CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

Embodiment 60 comprises the recombinant antibody or antigen binding fragment thereof of any one of embodiments 47-59, wherein the immunoglobulin light chain of the CD3 binding domain comprises a variable domain of an immunoglobulin kappa (IgK) or immunoglobulin lambda (IgL) light chain.

Embodiment 61 comprises the recombinant antibody or antigen binding fragment thereof of any one of embodiments 47-60, wherein the immunoglobulin heavy chain of the CD3 binding domain comprises a variable domain of an IgG1, IgG2, IgG3, or IgG4 heavy chain.

Embodiment 62 comprises the recombinant antibody or antigen binding fragment thereof of any one of embodiments 47-48, wherein the immunoglobulin light chain of the CD3 binding domain comprises an amino acid sequence with at least 90%, 95%, 96%, 97%, 98%, 99%, or 100% identity to SEQ ID NO: 81.

Embodiment 63 comprises the recombinant antibody or antigen binding fragment thereof of any one of embodiments 47-48, wherein the immunoglobulin heavy chain of the CD3 binding domain comprises an amino acid sequence with at least 90%, 95%, 96%, 97%, 98%, 99%, or 100% identity to SEQ ID NO: 82.

Embodiment 64 comprises the recombinant antibody or antigen binding fragment thereof of any one of embodiments 47-48, wherein the immunoglobulin light chain of the CD3 binding domain comprises an amino acid sequence with at least 90%, 95%, 96%, 97%, 98%, 99%, or 100% identity to SEQ ID NO: 81 and the immunoglobulin heavy chain of the CD3 binding domain comprises an amino acid sequence with at least 90%, 95%, 96%, 97%, 98%, 99%, or 100% identity to SEQ ID NO: 82.

Embodiment 65 comprises the recombinant antibody or antigen binding fragment thereof of any one of embodiments 47-64, wherein the CD3 binding domain comprises a Fab, Fab′, (Fab′)₂ or a single chain variable fragment (scFv).

Embodiment 66 comprises the recombinant antibody or antigen binding fragment thereof of embodiment 65, wherein the CD3 binding domain is the scFv.

Embodiment 67 comprises the recombinant antibody or antigen binding fragment thereof of any one of embodiments 47-48, wherein the CD3 binding domain comprises an amino acid sequence with at least 90%, 95%, 96%, 97%, 98%, 99%, or 100% identity to SEQ ID NO: 99.

Embodiment 68 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 131.

Embodiment 69 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 132.

Embodiment 70 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 133.

Embodiment 71 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 134.

Embodiment 72 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 135.

Embodiment 73 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 136.

Embodiment 74 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 137.

Embodiment 75 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 138.

Embodiment 76 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 139.

Embodiment 77 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 140.

Embodiment 78 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 141.

Embodiment 79 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 142.

Embodiment 80 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 143.

Embodiment 81 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 144.

Embodiment 82 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 145.

Embodiment 83 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 146.

Embodiment 84 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 147.

Embodiment 85 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 148.

Embodiment 86 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 149.

Embodiment 87 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 150.

Embodiment 88 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 151.

Embodiment 89 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 152.

Embodiment 90 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 153.

Embodiment 91 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 154.

Embodiment 92 comprises the recombinant antibody or antigen binding fragment thereof of embodiment 49 or 59, wherein the CD3 binding domain comprises an amino acid sequence with at least 90%, 95%, 96%, 97%, 98%, 99%, or 100% identity to SEQ ID NO: 155.

Embodiment 93 comprises the recombinant antibody or antigen binding fragment thereof of any one of embodiments 48-92, wherein the N-term of the immunoglobulin heavy chain of the TROP2 binding domain is bound to the C-term of the immunoglobulin light chain of the CD3 binding domain.

Embodiment 94 comprises the recombinant antibody or antigen binding fragment thereof of any one of embodiments 48-92, wherein the C-term of the immunoglobulin heavy chain of the TROP2 binding domain is bound to the N-term of the immunoglobulin light chain of the CD3 binding domain.

Embodiment 95 comprises the recombinant antibody or antigen binding fragment thereof of any one of embodiments 48-92, wherein the N-term of the immunoglobulin heavy chain of the TROP2 binding domain is bound to the C-term of the immunoglobulin heavy chain of the CD3 binding domain.

Embodiment 96 comprises the recombinant antibody or antigen binding fragment thereof of any one of embodiments 48-92, wherein the C-term of the immunoglobulin heavy chain of the TROP2 binding domain is bound to the N-term of the immunoglobulin heavy chain of the CD3 binding domain.

Embodiment 97 comprises the recombinant antibody or antigen binding fragment thereof of any one of embodiments 48-92, wherein the N-term of the immunoglobulin light chain of the TROP2 binding domain is bound to the C-term of the immunoglobulin light chain of the CD3 binding domain.

Embodiment 98 comprises the recombinant antibody or antigen binding fragment thereof of any one of embodiments 48-92, wherein the C-term of the immunoglobulin light chain of the TROP2 binding domain is bound to the N-term of the immunoglobulin light chain of the CD3 binding domain.

Embodiment 99 comprises the recombinant antibody or antigen binding fragment thereof of any one of embodiments 48-92, wherein the N-term of the immunoglobulin light chain of the TROP2 binding domain is bound to the C-term of the immunoglobulin heavy chain of the CD3 binding domain.

Embodiment 100 comprises the recombinant antibody or antigen binding fragment thereof of any one of embodiments 48-92, wherein the C-term of the immunoglobulin light chain of the TROP2 binding domain is bound to the N-term of the immunoglobulin heavy chain of the CD3 binding domain.

Embodiment 101 comprises the recombinant antibody or antigen binding fragment thereof of any one of embodiments 48-100, wherein the CD3 binding domain is a scFv and the TROP2 binding domain is a Fab or Fab′.

Embodiment 102 comprises the recombinant antibody or antigen binding fragment thereof of embodiment 101, wherein the scFv is bound to the immunoglobulin heavy chain of the Fab or Fab′.

Embodiment 103 comprises the recombinant antibody or antigen binding fragment thereof of embodiment 101, wherein the scFv is bound to the immunoglobulin light chain of the Fab or Fab′.

Embodiment 104 comprises the recombinant antibody or antigen binding fragment thereof of embodiment 101, wherein the immunoglobulin light chain of the scFv is bound to the immunoglobulin heavy chain of the Fab or Fab′.

Embodiment 105 comprises the recombinant antibody or antigen binding fragment thereof of embodiment 101, wherein the immunoglobulin light chain of the scFv is bound to the immunoglobulin light chain of the Fab or Fab′.

Embodiment 106 comprises the recombinant antibody or antigen binding fragment thereof of embodiment 101, wherein the immunoglobulin heavy chain of the scFv is bound to the immunoglobulin heavy chain of the Fab or Fab′.

Embodiment 107 comprises the recombinant antibody or antigen binding fragment thereof of embodiment 101, wherein the immunoglobulin heavy chain of the scFv is bound to the immunoglobulin light chain of the Fab or Fab′.

Embodiment 108 comprises the recombinant antibody or antigen binding fragment thereof of any one of embodiments 1, 15, and 47, wherein the recombinant antibody or antigen binding fragment thereof comprises amino acid sequences with at least 90%, 95%, 96%, 97%, 98%, 99%, or 100% identity to SEQ ID NO: 87 and SEQ ID NO: 88.

Embodiment 109 comprises the recombinant antibody or antigen binding fragment thereof of any one of embodiments 1, 15, and 47, wherein the recombinant antibody or antigen binding fragment thereof comprises amino acid sequences with at least 90%, 95%, 96%, 97%, 98%, 99%, or 100% identity to SEQ ID NO: 89 and SEQ ID NO: 90.

Embodiment 110 comprises the recombinant antibody or antigen binding fragment thereof of any one of embodiments 1, 15, and 47, wherein the recombinant antibody or antigen binding fragment thereof comprises amino acid sequences with at least 90%, 95%, 96%, 97%, 98%, 99%, or 100% identity to SEQ ID NO: 91 and SEQ ID NO: 92.

Embodiment 111 comprises the recombinant antibody or antigen binding fragment thereof of any one of embodiments 1, 15, and 47, wherein the recombinant antibody or antigen binding fragment thereof comprises amino acid sequences with at least 90%, 95%, 96%, 97%, 98%, 99%, or 100% identity to SEQ ID NO: 93 and SEQ ID NO: 94.

Embodiment 112 comprises the recombinant antibody or antigen binding fragment thereof of any one of embodiments 1, 15, and 47, wherein the recombinant antibody or antigen binding fragment thereof comprises amino acid sequences with at least 90%, 95%, 96%, 97%, 98%, 99%, or 100% identity to SEQ ID NO: 95 and SEQ ID NO: 96.

Embodiment 113 comprises the recombinant antibody or antigen binding fragment thereof of any one of embodiments 1, 15, and 47, wherein the recombinant antibody or antigen binding fragment thereof comprises amino acid sequences with at least 90%, 95%, 96%, 97%, 98%, 99%, or 100% identity to SEQ ID NO: 97 and SEQ ID NO: 98.

Embodiment 114 comprises the recombinant antibody or antigen binding fragment thereof of any one of embodiments 1, 15, 47, 49, and 59, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 188.

Embodiment 115 comprises the recombinant antibody or antigen binding fragment thereof of any one of embodiments 1, 15, 47, 49, and 59, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 189.

Embodiment 116 comprises the recombinant antibody or antigen binding fragment thereof of any one of embodiments 1, 15, 47, 49, and 59, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 190.

Embodiment 117 comprises the recombinant antibody or antigen binding fragment thereof of any one of embodiments 1, 15, 47, 49, and 59, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 194.

Embodiment 118 comprises the recombinant antibody or antigen binding fragment thereof of any one of embodiments 1, 15, 47, 49, and 59, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 195.

Embodiment 119 comprises the recombinant antibody or antigen binding fragment thereof of any one of embodiments 1, 15, 47, 49, and 59, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 196.

Embodiment 120 comprises the recombinant antibody or antigen binding fragment thereof of embodiment 47, wherein the recombinant antibody or antigen binding fragment thereof has weaker cytotoxicity activity as compared to a recombinant antibody or antigen binding fragment thereof that comprises an immunoglobulin light chain according to SEQ ID NO: 83 or 85 and an immunoglobulin heavy chain according to SEQ ID NO: 84 or 86 as measured in an in vitro tumor cell killing assay under substantially similar assay conditions.

Embodiment 121 comprises a pharmaceutical composition comprising: (i) the recombinant antibody or antigen binding fragment thereof of any one of embodiments 1-120; and (ii) a pharmaceutically acceptable excipient.

Embodiment 122 comprises an isolated recombinant nucleic acid molecule encoding a polypeptide of the recombinant antibody or antigen binding fragment thereof of any one of embodiments 1-120.

Embodiment 123 comprises a method of treating a cancer in a subject in need thereof comprising administering to the subject the recombinant antibody or antigen binding fragment thereof of any one of embodiments 1-120.

Embodiment 124 comprises the method of embodiment 123, wherein the cancer comprises breast cancer, lung cancer, urothelial cancer, endometrial cancer, ovarian cancer, prostate cancer, pancreatic cancer, gastric cancer, colon cancer, head and neck cancer, or glioma.

Embodiment 125 comprises the method of embodiment 124, wherein the breast cancer comprises triple-negative breast cancer.

Embodiment 126 comprises the method of embodiment 124, wherein the lung cancer comprises non-small cell lung cancer.

### EXAMPLES

### Example 1: Alanine Scanning of TROP2 Binding Domain CDR3s

Alanine scanning of the TROP2 binding domain (TBD) was carried out in order to establish CDR3 related sequence activity relationships (SAR). Alanine scanning was accomplished by mutating each individual residue in the CDR3 light chain (LC) region and the CDR3 heavy chain (HC) region of the anti-TROP2 Fab starting sequence to alanine. The amino acid sequences of the non-mutated (or “wild-type”) and mutated TBD constructs are shown in Table 3. TBD-1 is the “wild-type” or starting sequence anti-TROP2 Fab. TBD-2 to TBD-10 are the TBDs having individual alanine mutations in the CDR3 LC region, and TBD-11 to TBD-23 are the TBDs having individual alanine mutations in the CDR3 HC region.

### Example 2: Binding of TBDs to TROP2

### Kinetic Binding to TROP2 Via BLI

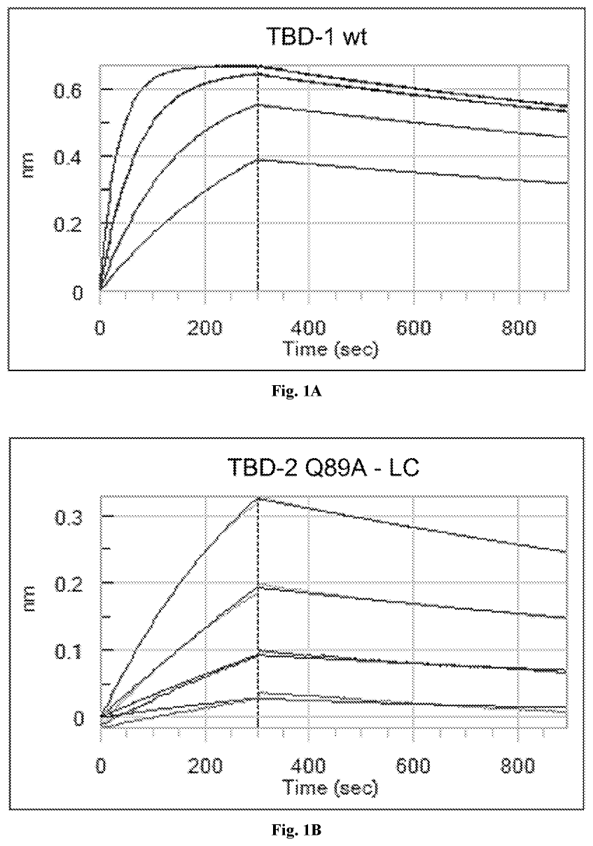

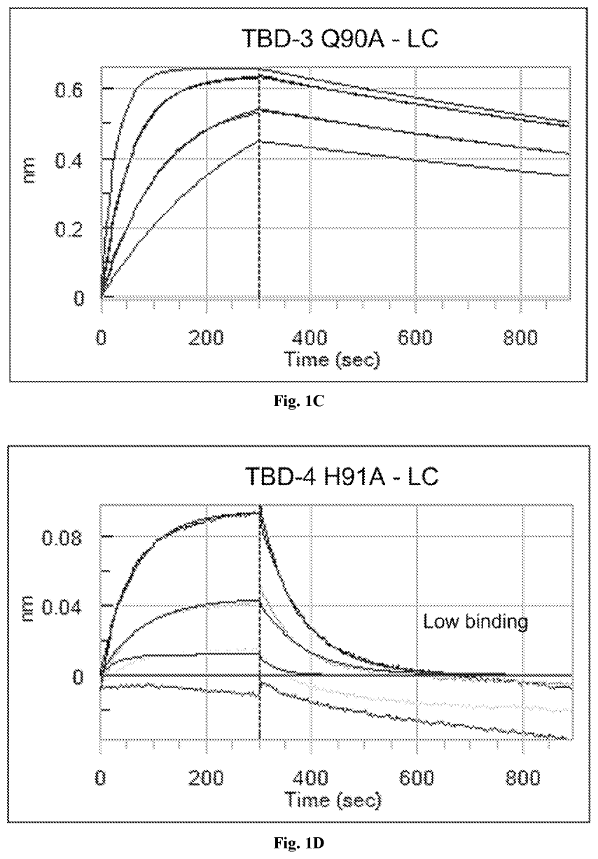

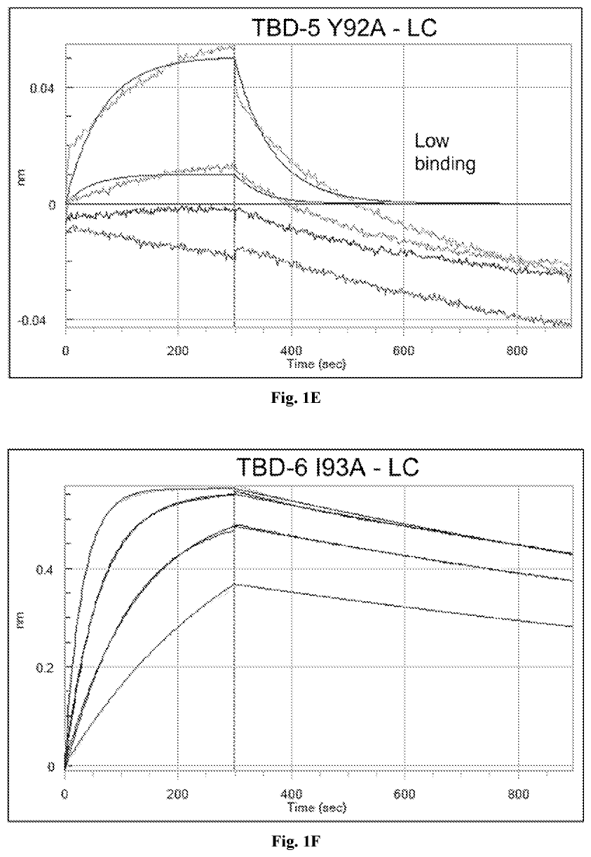

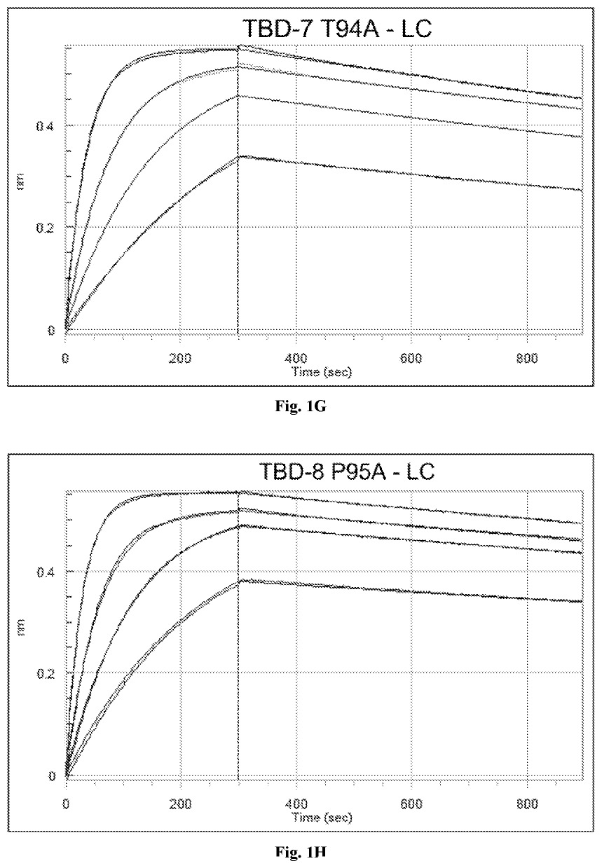

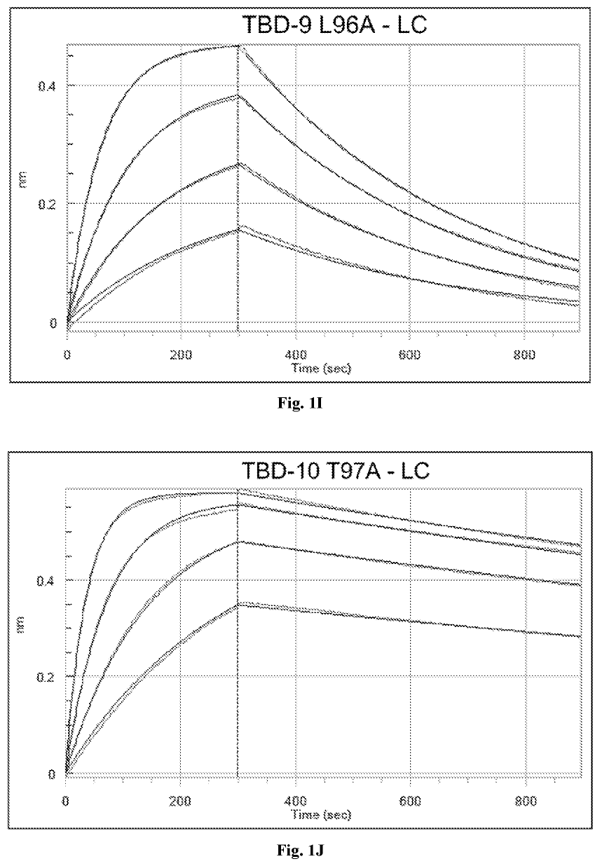

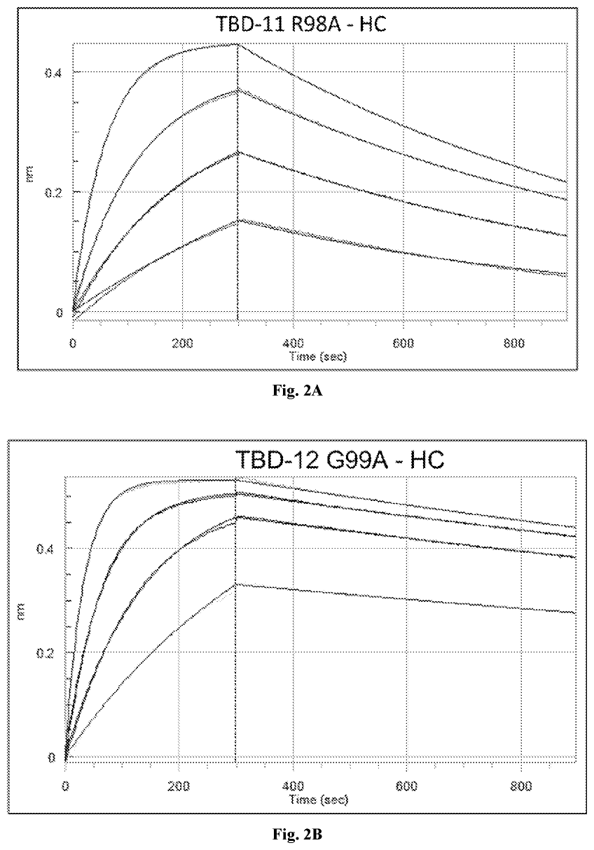

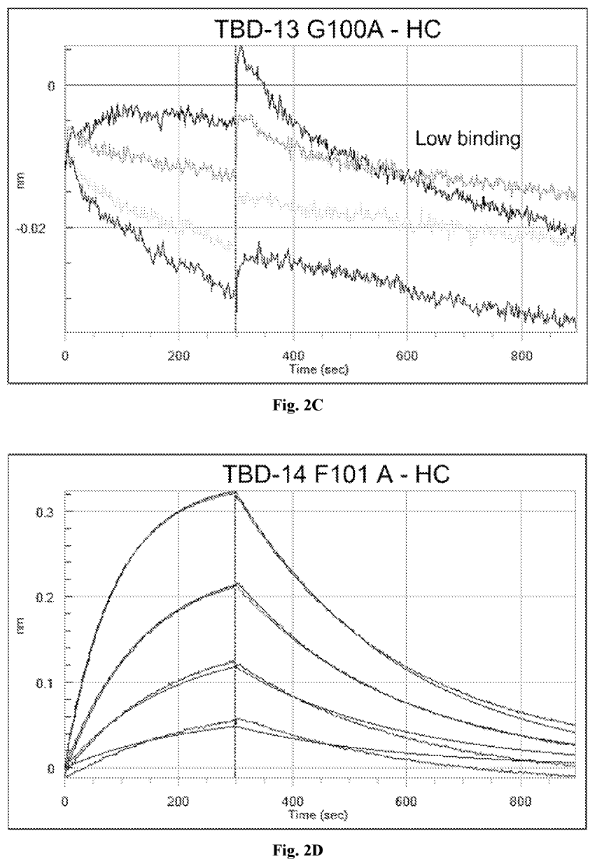

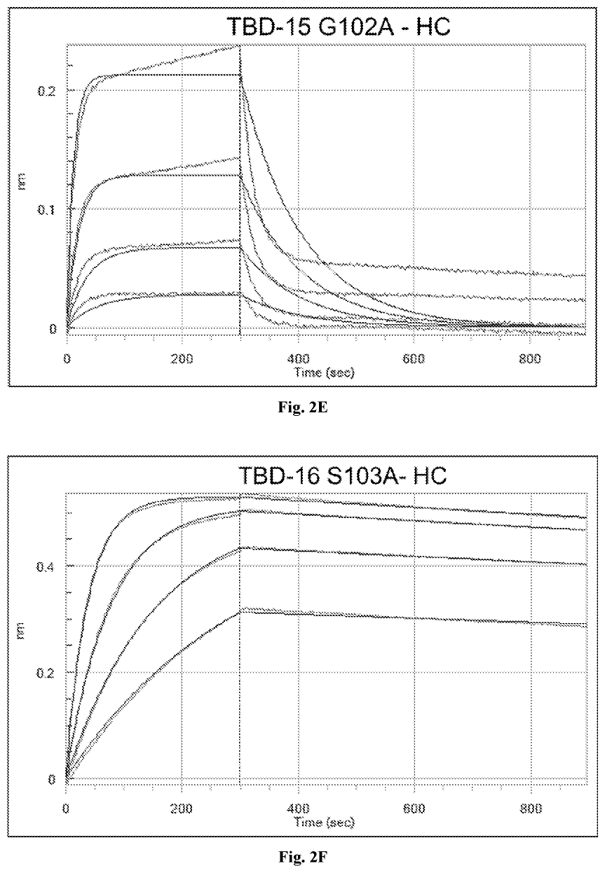

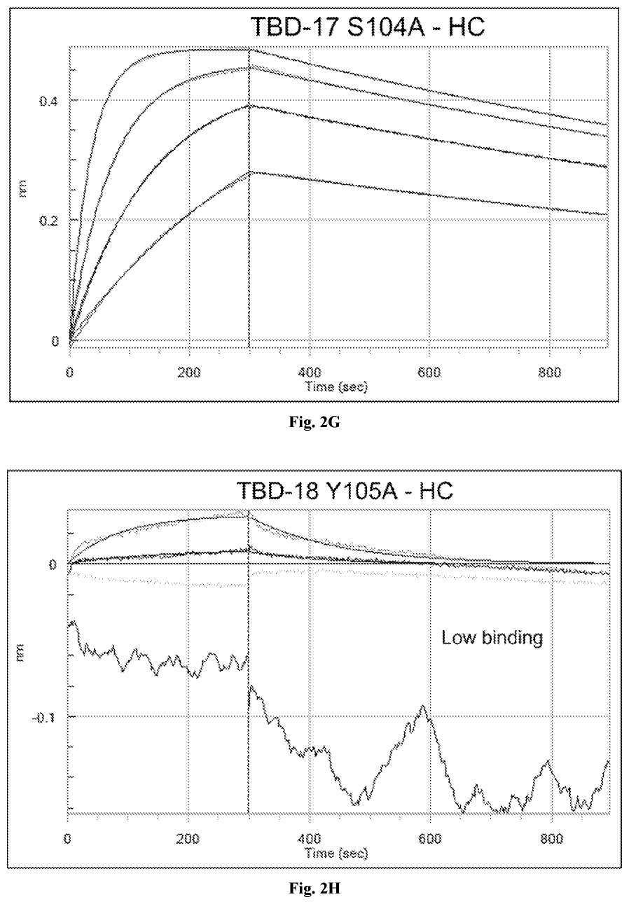

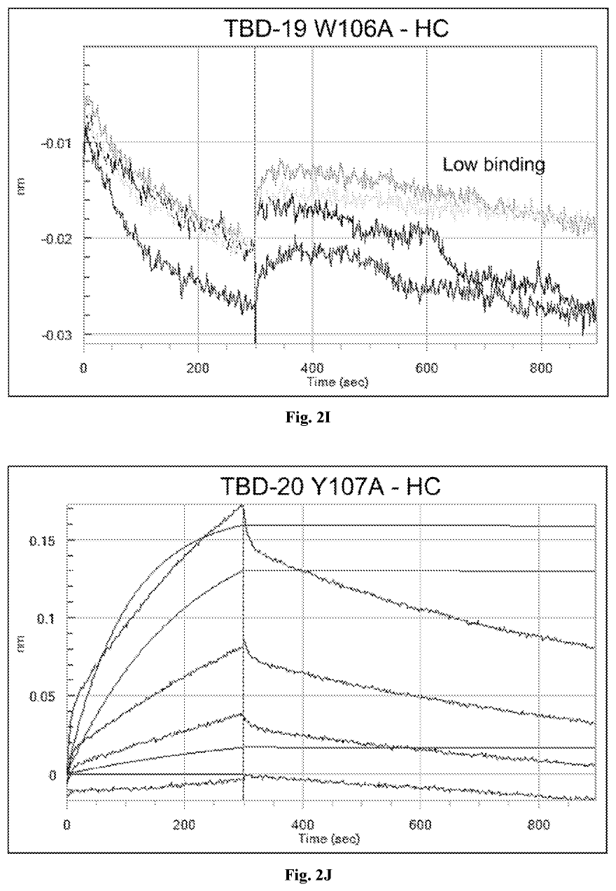

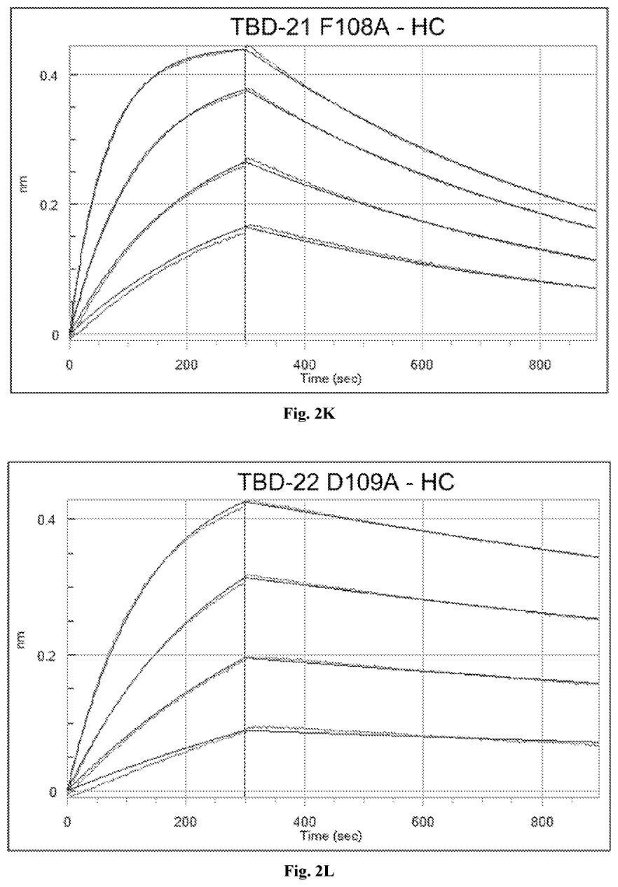

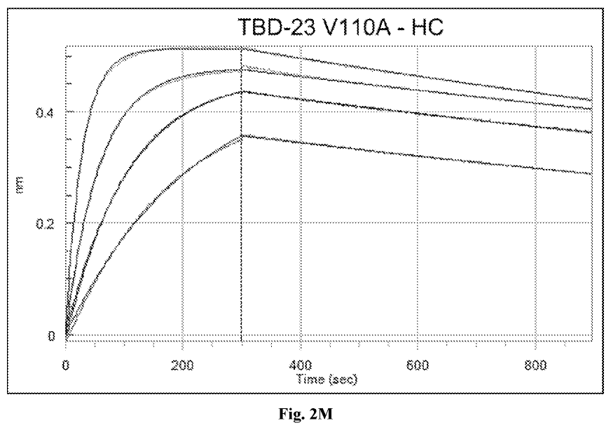

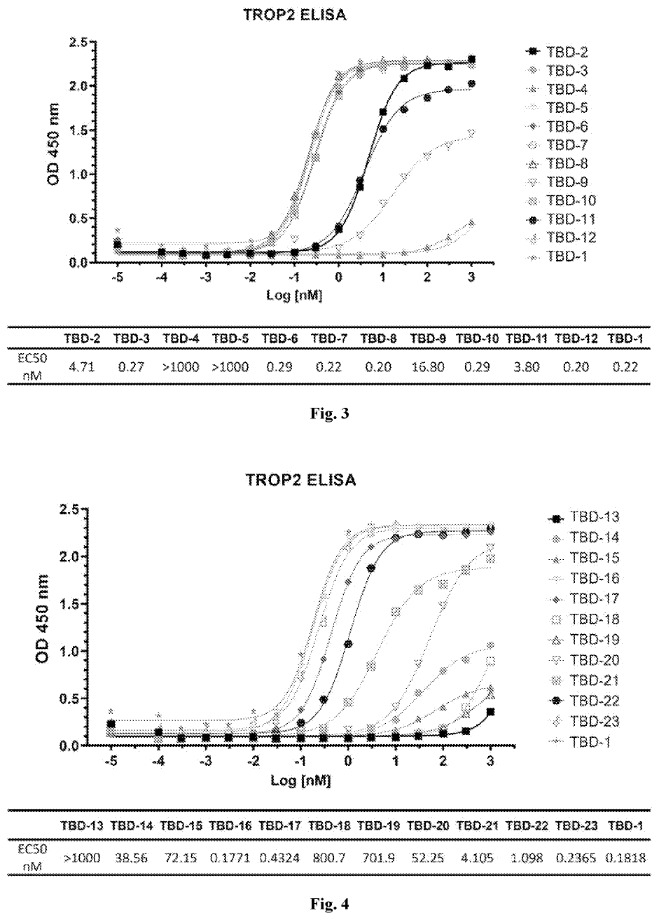

The wild-type and alanine mutated TBDs of Example 1 were evaluated for their ability to bind TROP2. Kinetic binding was measured using biolayer interferometry (BLI). Briefly, biotinylated human TROP2 was loaded onto a streptavidin coated Octet® SAX biosensor, quenched in biocytin, and baselined in buffer. The TBDs diluted in buffer were then associated onto the antigen loaded biosensors. Sensors were then transferred to buffer where the TBD constructs then dissociated from the sensors. Association and dissociation rates were measured in real time using an Octet® instrument. Example sensorgrams for non-mutated (TBD-1) and CDR3 LC alanine scanning mutants (TBD-2 to TBD-10) are shown in FIGS. 1A-1J. Example sensorgrams for CDR3 HC alanine scanning mutants (TBD-11 to TBD-23) are shown in FIGS. 2A-2M. Exemplary experimental conditions and steps used for the kinetic binding measurements are shown in Table 9. For both the kinetic and equilibrium binding studies, the extracellular domain (ECD) of TROP2 was used (Table 10; SEQ ID NO: 100). The amino acid sequence of the extracellular domain of cynomolgus monkey TROP2 (SEQ ID NO: 101) as well as the frill length amino acid sequences for human and cynomolgus monkey TROP2 (SEQ ID NO: 102 and SEQ ID NO: 103) are also provided in Table 10.

TABLE 9

| Step | Time |
|---|---|
| Sensor: SAX | |
| Baseline: Octet buffer | 60 seconds |
| Loading: Human | 300 seconds |
| TROP2-biotin (10 nM) | |
| Biocytin quench | 300 seconds |
| (100 μM) | |
| Baseline: Octet buffer | 300 seconds |
| Association: | 300 seconds |
| 50 nM Fab | |
| 25 nM Fab | |
| 12.5 nM Fab | |
| 6.25 nM Fab | |
| Dissociation: Octet buffer | 600 seconds |

TABLE 10
Human and cynomolgus monkey TROP2 sequences.

| | | SEQ ID |
|---|---|---|
| Description | Amino Acid Sequence (N to C) | NO: |
| Human TROP2 | QDNCTCPTNKMTVCSPDGPGGRCQCRALGSGMAVD | 100 |
| ECD (31-274 | CSTLTSKCLLLKARMSAPKNARTLVRPSEHALVDNDGLY | |
| AA) | DPDCDPEGRFKARQCNQTSVCWCVNSVGVRRTDKGD | |
| | LSLRCDELVRTHHILIDLRHRPTAGAFNHSDLDAELRRLF | |
| | RERYRLHPKFVAAVHYEQPTIQIELRQNTSQKAAGDVDI | |
| | GDAAYYFERDIKGESLFQGRGGLDLRVRGEPLQVERTLI | |
| | YYLDEIPPKFSMKRLT | |
| Cyno TROP2 | QDNCTCPTNKMTVCSPDGPGGRCQCRALGSGVAVDC | 101 |
| ECD (31-274 | STLTSKCLLLKARMSAPKNARTLVRPNEHALVDNDGLY | |
| AA) | DPDCDPEGRFKARQCNQTSVCWCVNSVGVRRTDKGD | |
| | LSLRCDELVRTHHILIDLRHRPTAGAFNHSDLDAELRRLF | |
| | RERYRLHPKFVAAVHYEQPTIQIELRQNTSQKAAGDVDI | |
| | GDAAYYFERDVKGESLFQGRGGLDLRVRGEPLQVERTLI | |
| | YYLDEIPPKFSMKRLT | |
| Human TROP2 | QDNCTCPTNKMTVCSPDGPGGRCQCRALGSGMAVDCSTL | 102 |
| Full length | TSKCLLLKARMSAPKNARTLVRPSEHALVDNDGLYDPDCDPE | |
| | GRFKARQCNQTSVCWCVNSVGVRRTDKGDLSLRCDELVRT | |
| | HHILIDLRHRPTAGAFNHSDLDAELRRLFRERYRLHPKFVAAV | |
| | HYEQPTIQIELRQNTSQKAAGDVDIGDAAYYFERDIKGESLF | |
| | QGRGGLDLRVRGEPLQVERTLIYYLDEIPPKFSMKRLTAGLIA | |
| | VIVVVVVALVAGMAVLVITNRRKSGKYKKVEIKELGELRKEPSL | |
| Cyno TROP2 | QDNCTCPTNKMTVCSPDGPGGRCQCRALGSGVAVDCSTLT | 103 |
| Full length | SKCLLLKARMSAPKNARTLVRPNEHALVDNDGLYDPDCDPE | |
| | GRFKARQCNQTSVCWCVNSVGVRRTDKGDLSLRCDELVRT | |
| | HHILIDLRHRPTAGAFNHSDLDAELRRLFRERYRLHPKFVAAV | |
| | HYEQPTIQIELRQNTSQKAAGDVDIGDAAYYFERDVKGESLF | |
| | QGRGGLDLRVRGEPLQVERTLIYYLDEIPPKFSMKRLTAGLIA | |
| | VIVVVVVALVAGVAVLVISNRRKSGKYKKVEIKELGELRKEPSL | |

Equilibrium binding to TROP2 via ELISA

The wild-type and alanine mutated TBDs of Example 1 were also evaluated for their ability to bind TROP2 using a standard enzyme-linked immunosorbent assay (ELISA) format. Briefly, biotinylated TROP2 was captured on neutravidin coated plates. The TROP2 Fab constructs (TBDs) diluted in buffer were then added to the antigen coated plates. Binding was detected using a standard horse radish peroxidase secondary antibody. The concentration of the TBD required to achieve 50% maximal signal (EC₅₀) was calculated. Binding curves for the TBD CDR3 LC and CDR3 HC alanine scanning mutants (TBD-2 to TBD-12) relative to the wild-type TBD (TBD-1) are shown in FIG. 3 along with calculated EC₅₀s. Binding curves for CDR3 HC alanine scanning mutants (TBD-13 to TBD-23) relative to the wild-type TBD (TBD-1) are shown in FIG. 4 along with calculated EC₅₀s.

### Sequence Activity Relationships

Data summarizing the TROP2 binding parameters obtained from the above kinetic and equilibrium binding studies are provided in Table 11 (CDR3 LC alanine mutated constructs) and Tables 12-13 (CDR3 HC alanine mutated constructs). The tables also designate each alanine mutation in the CDR3 LC and CDR3 HC regions as tolerated (T), moderately tolerated (MT), or not tolerated (NT). Alanine mutations which resulted in significantly weakened binding relative to the non-mutated TROP2 Fab (TBD-1) were characterized as NT, and those residues were considered key residues for TROP2 binding. Alanine mutations which showed comparable binding parameters to the non-mutated TROP2 Fab were characterized as T, and those residues were considered non-critical for TROP2 binding. Alanine mutations which resulted in somewhat weakened binding relative to the non-mutated TROP2 Fab were characterized as MT.

As can be seen from Table 11, the H91A and Y92A mutations in the CDR3 LC region were not tolerated and weakened the binding interaction with TROP2 substantially. Also disruptive to the TROP2 binding interaction was the L96A mutation. The Q89A mutation was moderately tolerated, whereas the remaining alanine mutations in the CDR3 LC region were tolerated. The data indicates that H91A and Y92A of the CDR3 LC region are critical for binding to TROP2.

Regarding the alanine mutations in the CDR3 HC region, the G100A, Y105A, and W106A mutations were not tolerated and disrupted the binding interaction with TROP2 substantially, indicating that these residues are critical for binding. The F101A and G102A mutations were also significantly disruptive to binding, suggesting that these residues are also important for the binding interaction with the TROP2 antigen.

TABLE 11
TROP2 Binding and SAR Data for TROP2 Fab CDR3 LC Alanine Scanning Mutants

| TROP2 | TBD1 | | | | | | | | | |
|---|---|---|---|---|---|---|---|---|---|---|
| Fab | wt | TBD2 | TBD3 | TBD4 | TBD5 | TBD6 | TBD7 | TBD8 | TBD9 | TBD10 |
| K_{D} (nM) | 0.6 | 10.4 | 0.7 | low | low | 0.7 | 0.6 | 0.3 | 8.4 | 0.6 |
| | | | | binding | binding | | | | | |
| t_{1/2} (min) | 50 | 24 | 27 | — | — | 28 | 39 | 61 | 5 | 37 |
| EC₅₀ - ELISA (nM) | 0.2 | 4.7 | 0.3 | >1000 | >1000 | 0.3 | 0.2 | 0.2 | 16.8 | 0.3 |
| Fold shift in EC₅₀ | N/A | 23.5 | 1.5 | ND | ND | 1.5 | 1 | 1 | 84 | 1.5 |
| relative to wt | | | | | | | | | | |
| CDR3 LC Ala scan | N/A | Q | Q | H | Y | I | T | P | L | T |
| Amino acid position | N/A | 89 | 90 | 91 | 92 | 93 | 94 | 95 | 96 | 97 |
| Mutation tolerated? | N/A | MT | T | NT | NT | T | T | T | NT | T |

TABLE 12
TROP2 Binding and SAR Data for TROP2 Fab CDR3 HC Alanine Scanning Mutants

| | TBD1 | | | | | | | |
|---|---|---|---|---|---|---|---|---|
| TROP2 Fab | wt | — | TBD11 | TBD12 | TBD13 | TBD14 | TBD15 | TBD16 |
| K_{D} (nM) | 0.6 | | 3.88 | 0.492 | low | 18.7 | 5.71 | 0.211 |
| | | | | | binding | | | |
| t_{1/2} (min) | 50 | | 10 | 39 | — | 4 | 1 | 103 |
| EC₅₀ - ELISA (nM) | 0.2 | | 3.8 | 0.2 | >1000 | 38.6 | 72.2 | 0.2 |
| Fold shift in EC₅₀ | N/A | | 19 | 1 | ND | 193 | 361 | 1 |
| relative to wt | | | | | | | | |
| CDR3 LC Ala scan | N/A | A | R | G | G | F | G | S |
| Amino acid position | N/A | 97 | 98 | 99 | 100 | 101 | 102 | 103 |
| Mutation tolerated? | N/A | N/A | MT | T | NT | NT | NT | T |

TABLE 13
TROP2 Binding and SAR Data for TROP2 Fab CDR3 HC Alanine Scanning Mutants

| | TBD1 | | | | | | | |
|---|---|---|---|---|---|---|---|---|
| TROP2 Fab | wt | TBD17 | TBD18 | TBD19 | TBD20 | TBD21 | TBD22 | TBD23 |
| K_{D} (nM) | 0.6 | 0.9 | low | low | 6.34 | 4.76 | 2.35 | 0.401 |
| | | | binding | binding | | | | |
| t_{1/2} (min) | 50 | 24 | — | — | 10 | 8 | 33 | 34 |
| EC₅₀ - ELISA (nM) | 0.2 | 0.4 | 800.7 | 701.9 | 52.3 | 4.1 | 1.1 | 0.2 |
| Fold shift in EC₅₀ | N/A | 2 | 4004 | 3510 | 262 | 20.5 | 5.5 | 1 |
| relative to wt | | | | | | | | |
| CDR3 LC Ala scan | N/A | S | Y | W | Y | F | D | V |
| Amino acid position | N/A | 104 | 105 | 106 | 107 | 108 | 109 | 110 |
| Mutation tolerated? | N/A | T | NT | NT | MT | MT | MT | T |

### Example 3: Alanine Mutations of T-Cell Engager (TCE) Sequences

Selected alanine mutations in the TROP2 binding domain CDR3 HC region were carried out in TCE sequences composed of the TROP2 binding domain linked to a CD3ε binding domain. In these sequences, the CD3ε binding domain is an anti-CD3ε single chain variable fragment (scFv). The amino acid sequences of the non-mutated and mutated TCE constructs are provided in Table 7. TCE-1 and TCE-2 are non-mutated constructs having the CD3 binding domain connected to the TROP2 binding domain HC and the TROP2 binding domain LC, respectively. TCE-3 to TCE-5 include individual alanine mutations in the CDR3 HC region of the TROP2 binding domain and have the CD3 binding domain connected to the TROP2 binding domain LC. Specifically, TCE-3 includes a R98A mutation, TCE-4 includes a F108A mutation, and TCE-5 includes a D109A mutation. TCE-6 to TCE-8 include individual alanine mutations in the CDR3 HC region of the TROP2 binding domain and have the CD3 binding domain connected to the TROP2 binding domain HC. TCE-6 includes a R98A mutation, TCE-7 includes a F108A mutation, and TCE-8 includes a D109A mutation.

Surprisingly, Applicant has found that treatment of non-human primates with certain T-cell engager (TCE) constructs having single alanine mutations in the CD3 binding domain resulted in reduced levels of cytokine release compared to treatment with TCE constructs with a wild-type CD3 binding domain. Namely, the cytokine induction levels in non-human primates were lower for animals administered the maximum tolerated dose of CD3 mutant TCEs compared to animals administered with the maximum tolerated dose of wild-type CD3 TCEs. These results suggest reduced side effects related to cytokine release (e.g., cytokine release syndrome) after treatment with certain alanine mutated constructs. Additionally, the alanine mutated TCE constructs with weaker potencies may enable a wider therapeutic window between active dose levels and toxic dose levels relative to stronger potency wild-type constructs. This in turn could provide an advantage for standard dosing regimens where variability in population-based pharmacokinetics could be the difference between efficacy and toxicity. For example, controlling drug exposure is less challenging when there exists a wider drug exposure window between the maximum tolerated dose and the lethal dose. The difference in exposure between the non-human primate maximum tolerated dose and the lethal dose was larger for TCE constructs with reduced CD3 binding affinity and weaker in vitro cytotoxic potency. Further, the alanine mutated constructs with weaker potencies may also have benefits in analytics related to pharmacokinetic monitoring compared to stronger potency molecules which may be difficult to monitor at active doses when the detection limit of the analytical methods are above the drug exposure level. The weaker potency alanine mutated constructs disclosed herein may thus be easier to monitor clinically and advance in a clinical setting.

### Example 4: Binding of TROP-2 TCEs to TROP2 and CD3

### Kinetic Binding to TROP2 Via BLI

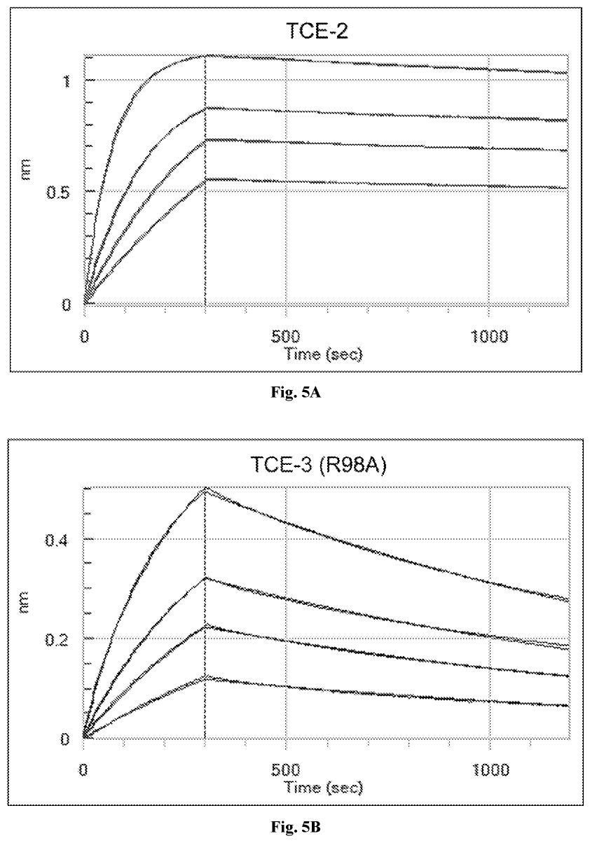

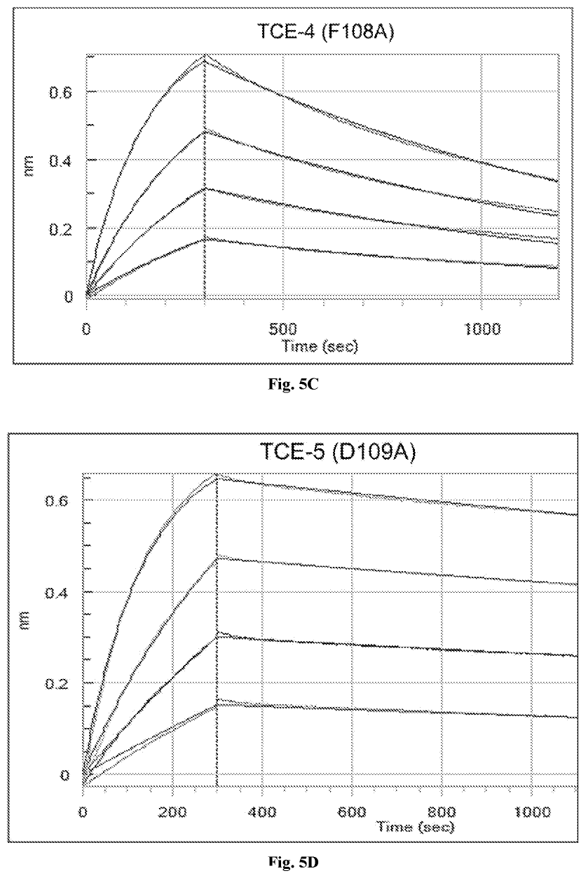

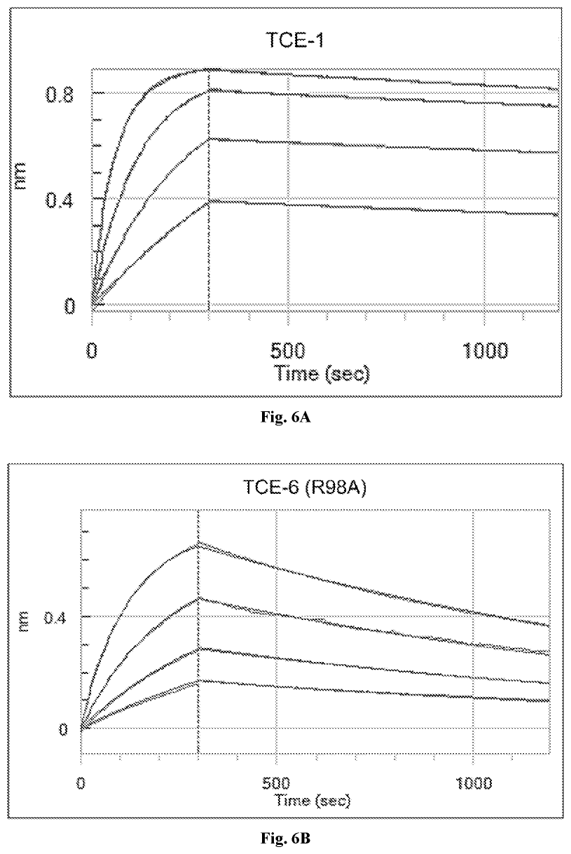

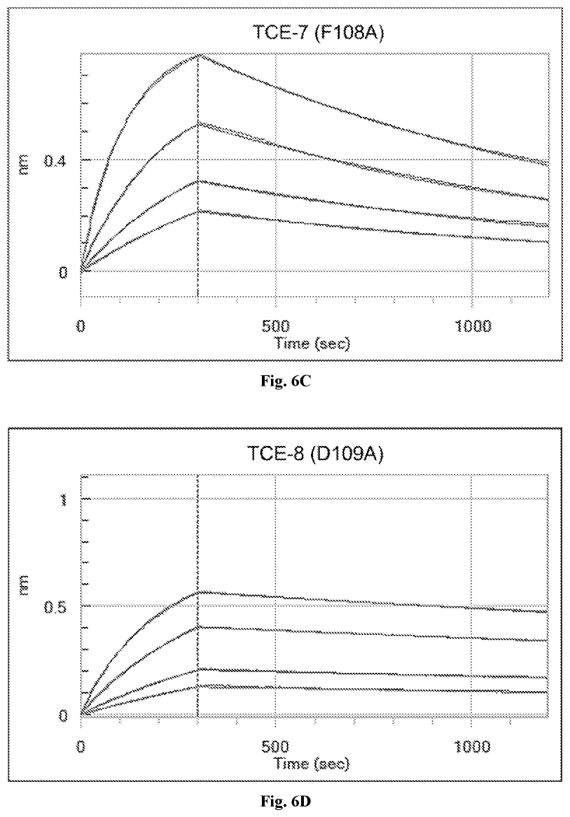

The non-mutated and alanine mutated TCEs of Example 3 were evaluated for their ability to bind TROP2. Binding kinetics was measured using BLI as described above in Example 2. Example sensorgrams for non-mutated construct TCE-2 and mutated constructs TCE-3 to TCE-5 are shown in FIGS. 5A-5D. Example sensorgrams for non-mutated construct TCE-1 and mutated constructs TCE-6 to TCE-8 are shown in FIGS. 6A-6D. Exemplary experimental conditions and steps used for the kinetic binding measurements are shown in Table 14.

TABLE 14

| Step | Time |
|---|---|
| Sensor: SAX | |
| Baseline: Octet buffer | 60 seconds |
| Loading: Human | 300 seconds |
| TROP2-biotin (10 nM) | |
| Biocytin quench | 300 seconds |
| (100 μM) | |
| Baseline: Octet buffer | 300 seconds |
| Association: | 300 seconds |
| 50 nM TCE | |
| 25 nM TCE | |
| 12.5 nM TCE | |
| 6.25 nM TCE | |
| Dissociation: Octet buffer | 600 seconds |

### Equilibrium Binding to TROP2 and CD3 Via ELISA

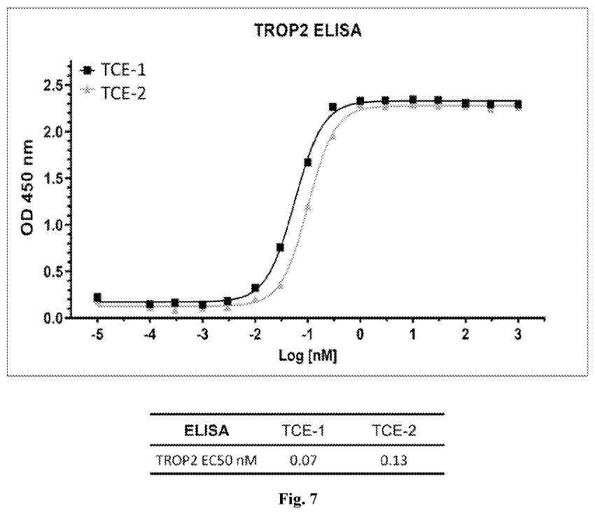

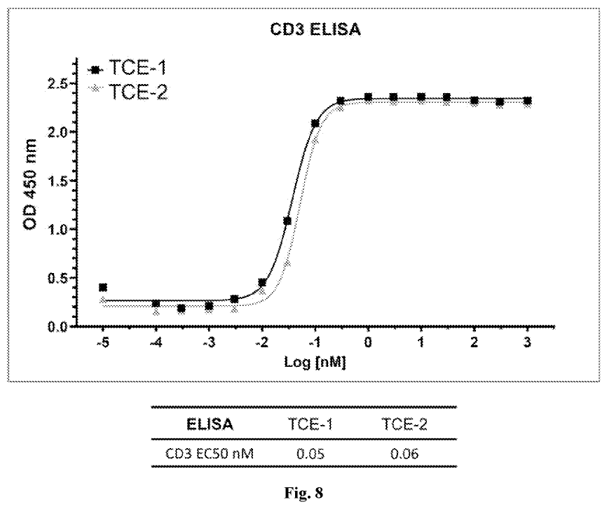

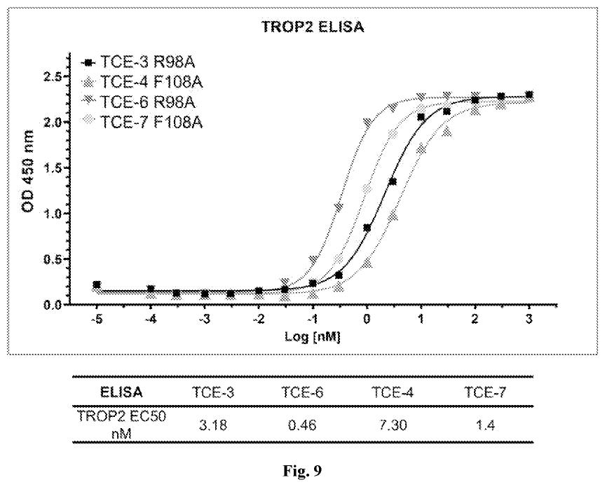

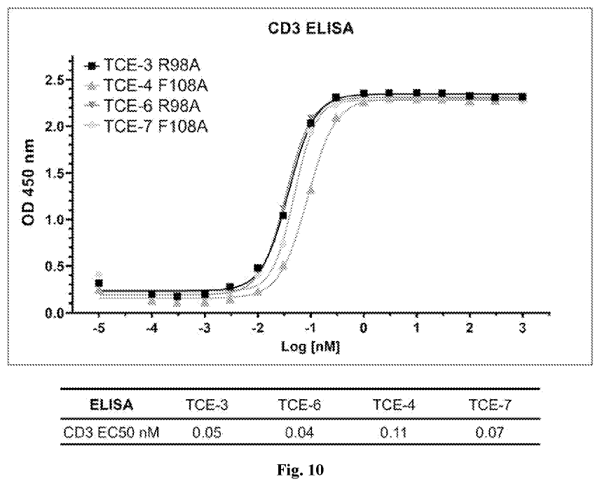

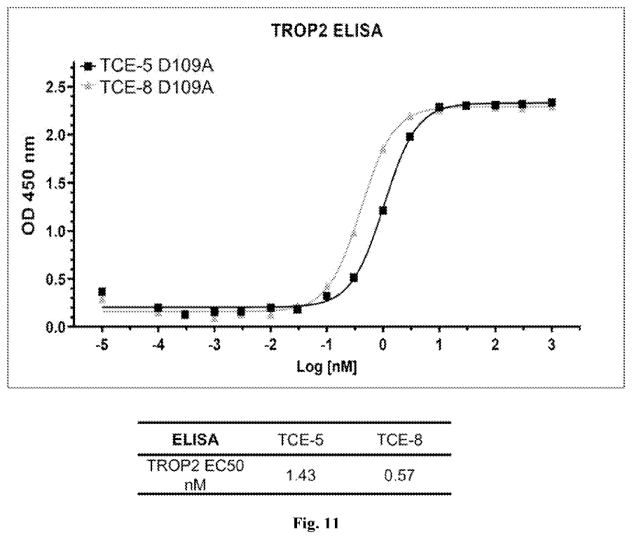

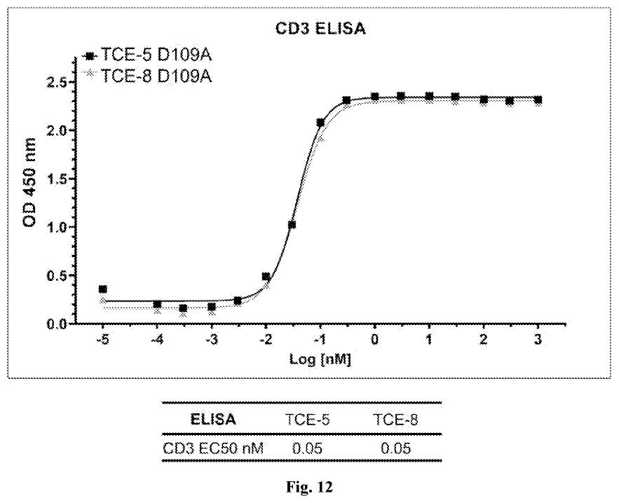

The non-mutated and alanine mutated TCEs of Example 3 were also evaluated for their ability to bind TROP2 and CD3c using an ELISA format. ELISA binding measurements were carried out as described in Example 2 using immobilized TROP2 or immobilized CD3ε. Binding curves and EC₅₀s for TROP2 and CD3ε binding by the non-mutated constructs TCE-1 and TCE-2 are shown in FIGS. 7-8. Binding curves and EC₅₀s for TROP2 and CD3ε binding by the mutated constructs TCE-3 to TCE-8 are shown in FIGS. 9-12. As can be seen, all mutations of the CDR3 HC region of the TBD resulted in increased EC₅₀s (weakened binding) for TROP2 relative to the non-mutated constructs. In contrast, mutations to the CDR3 HC region had minor or negligible effects on CD36 binding relative to the non-mutated constructs.

### Example 5: Tumor Cell Killing with TCEs

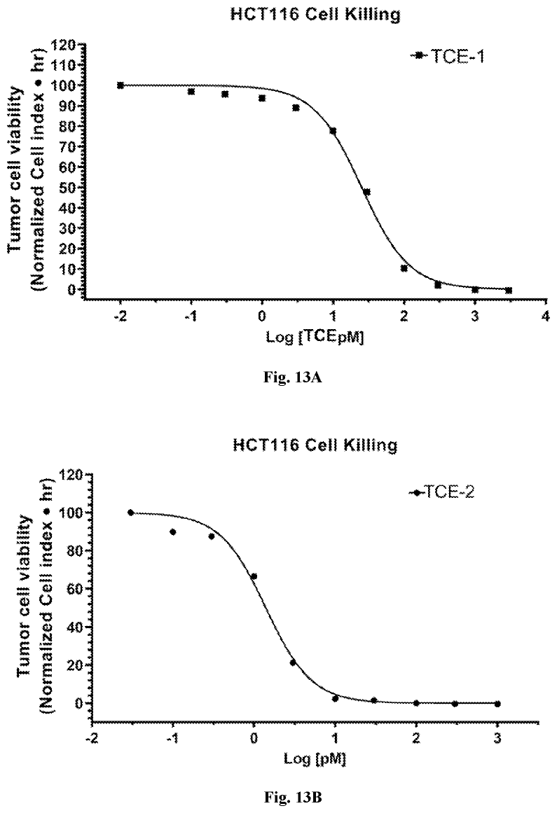

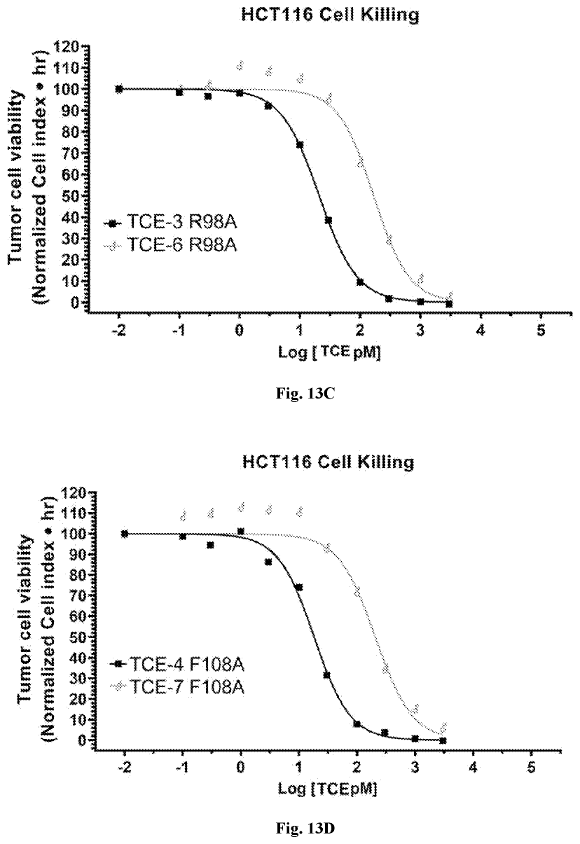

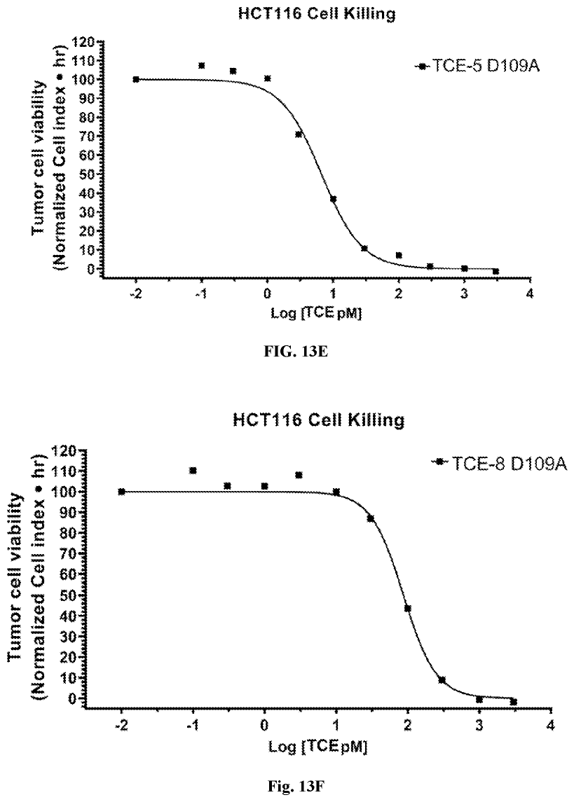

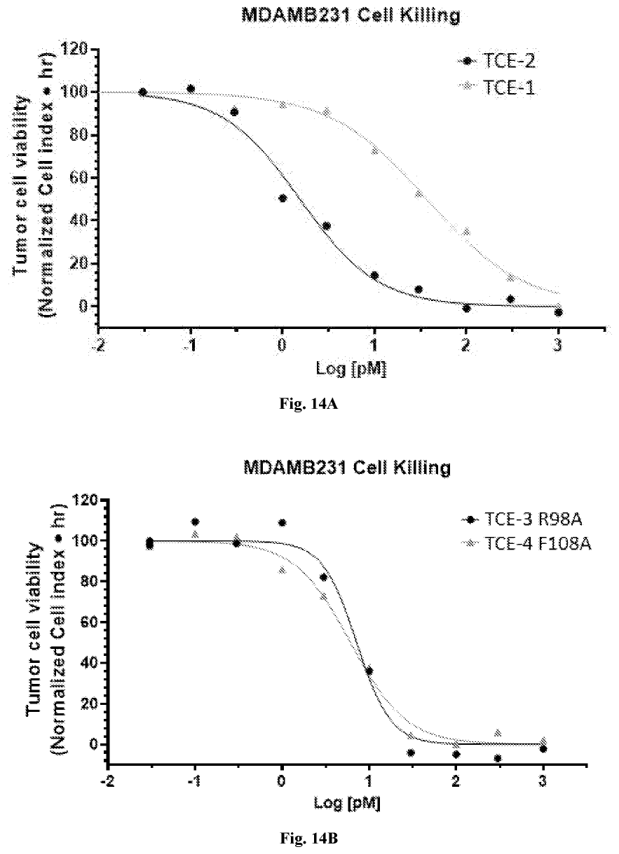

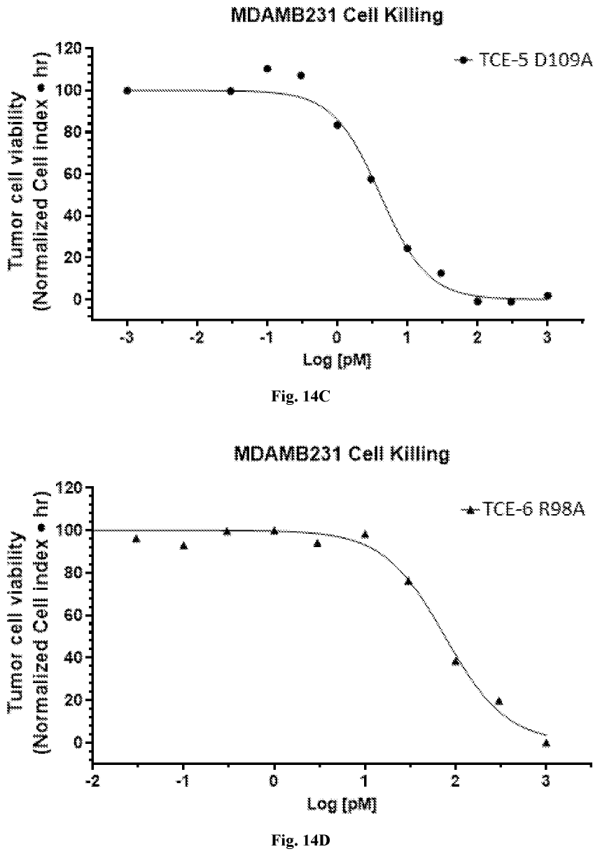

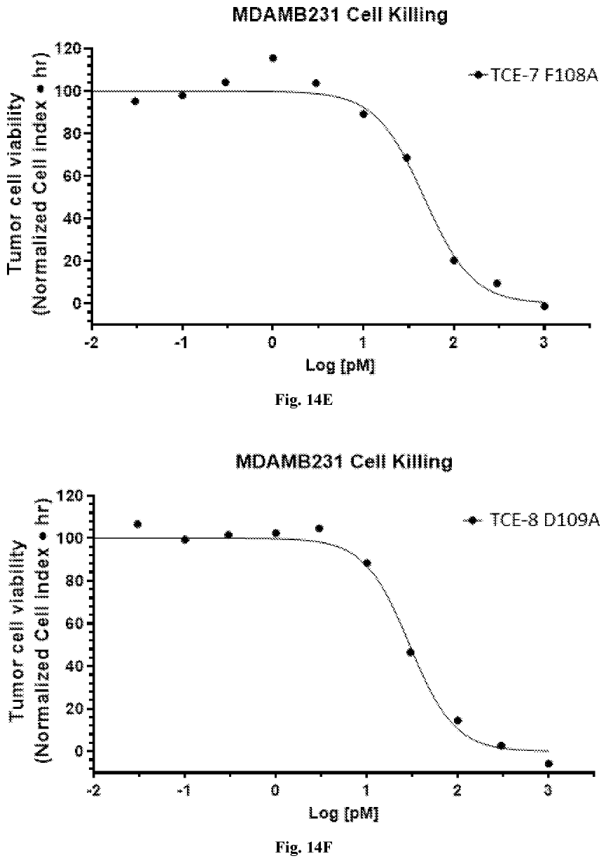

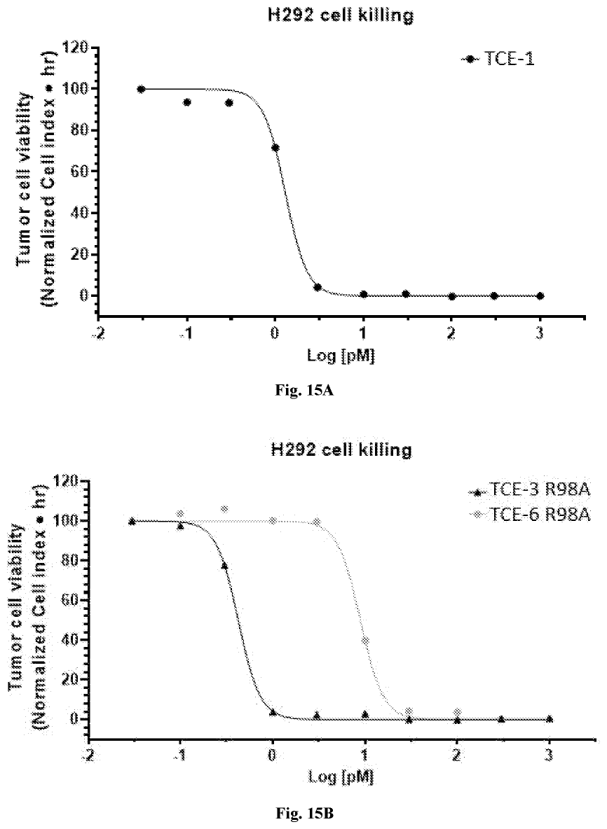

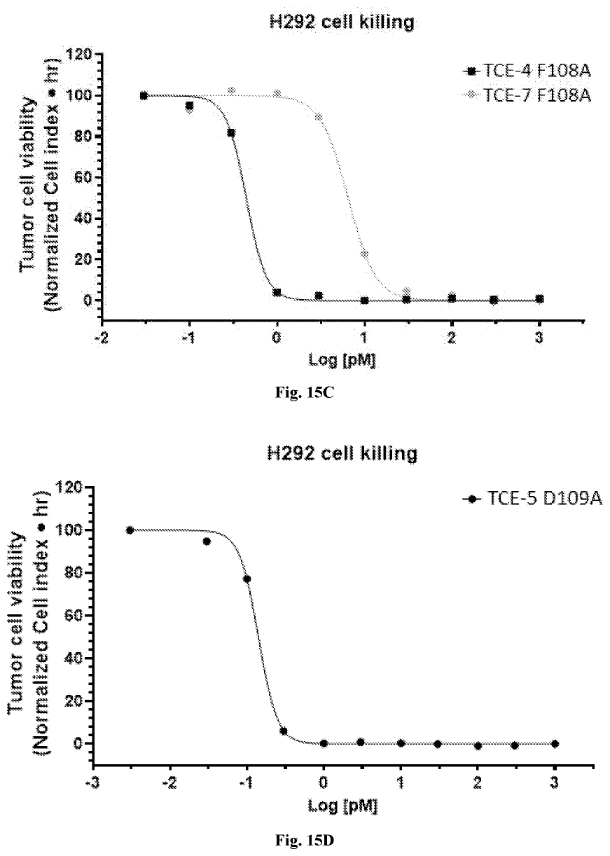

The TCEs of Example 3 were evaluated in a functional in vitro tumor cell killing assay using the TROP2 positive tumor cell lines HCT116, MDAMB231, and NCI-H292. Tumor cell killing was measured using an ×Celligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight. The following day, TCEs titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of TCE where the concentration required to reduce the tumor growth by 50% (EC₅₀) was calculated using Graphpad Prism software. Data plots of tumor cell viability vs. TCE concentration are shown in FIGS. 13A-13F (HCT116 cells), FIGS. 14A-14F (MDAMB231 cells), and FIGS. 15A-15E (NCI-H292 cells). The EC₅₀s for tumor cell killing of the different tumor cell types are provided in Table 15. The cells used for the cytotoxicity studies express the full length TROP2 antigen (see Table 10).

TABLE 15
TCE Cytotoxicity EC₅₀s

| TCE | HCT116 (pM) | MDAMB231 (pM) | H292 (pM) |
|---|---|---|---|
| TCE-1 | 28.6 | 12.0 | 1.3 |
| TCE-2 | 1.6 | 1.2 | — |
| TCE-3 | 22.0 | 7.7 | 0.4 |
| TCE-4 | 18.3 | 6.3 | 0.4 |
| TCE-5 | 5.8 | 2.6 | 0.2 |
| TCE-6 | 142.5 | 77.2 | 6.3 |
| TCE-7 | 205.8 | 47.8 | 5.6 |
| TCE-8 | 83.0 | 26.0 | 4.0 |

### Example 6: Alanine Scanning in TROP2 TCE Constructs

Alanine scanning of the light and heavy chains of anti-CD3 scFv CDR3s of the TROP2 T cell engager (TCE) constructs was carried out in order to establish anti-CD3 CDR3 related sequence activity relationships (SAR). Alanine scanning was accomplished by mutating each individual residue in the CDR3 light chain (LC) region and the CDR3 heavy chain (HC) region of the anti-CD3 scFv starting sequence to alanine. The amino acid sequences of the non-mutated (or “wild-type”) and mutated TCE constructs are provided in Table 8. TCE-9 has the “wild-type” or non-mutated anti-CD3 CDR3 sequences. TCE-10 to TCE-24 have individual alanine mutations in the anti-CD3 CDR3 heavy chain, and TCE-25 to TCE-34 have individual alanine mutations in the anti-CD3 CDR3 light chain. TCE-38 to TCE-40 and TCE-44 to TCE-46 include alanine mutations in both the anti-CD3 CDR3 and the anti-TROP2 CDR3 heavy chains (double mutants).

### Example 7: Binding of TROP2 TCEs to CD3ε and TROP2

### Equilibrium Binding to CD3ε and TROP2 by ELISA

The wild-type and alanine mutated TCEs of Example 6 were evaluated for their ability to bind CD36 and TROP2 using a standard enzyme-linked immunosorbent assay (ELISA) format. Briefly, biotinylated CD3ε or biotinylated TROP2 was captured on neutravidin coated plates. The TCE constructs diluted in buffer were then added to the antigen coated plates. Binding was detected using a standard horse radish peroxidase secondary antibody. The concentration of the TCE required to achieve 50% maximal signal (EC₅₀) was calculated.

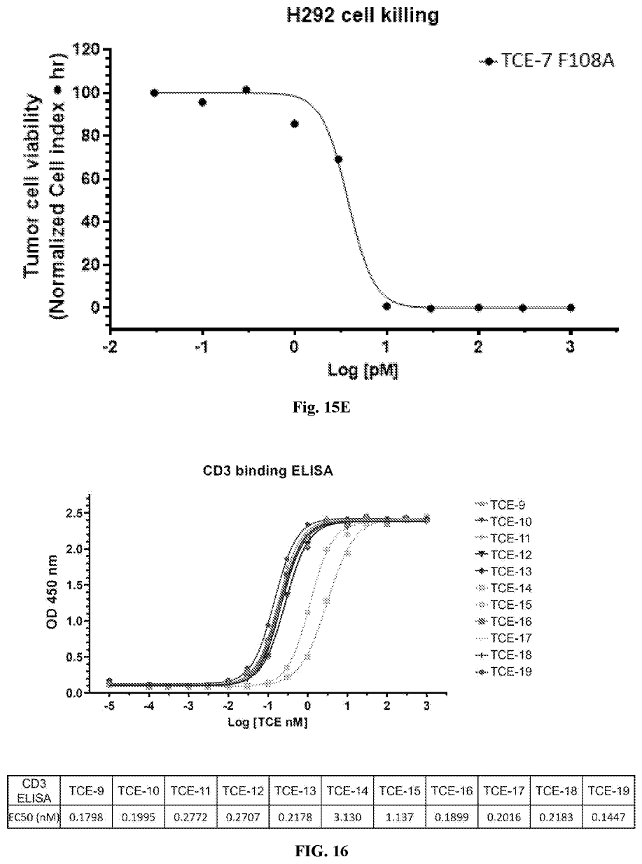

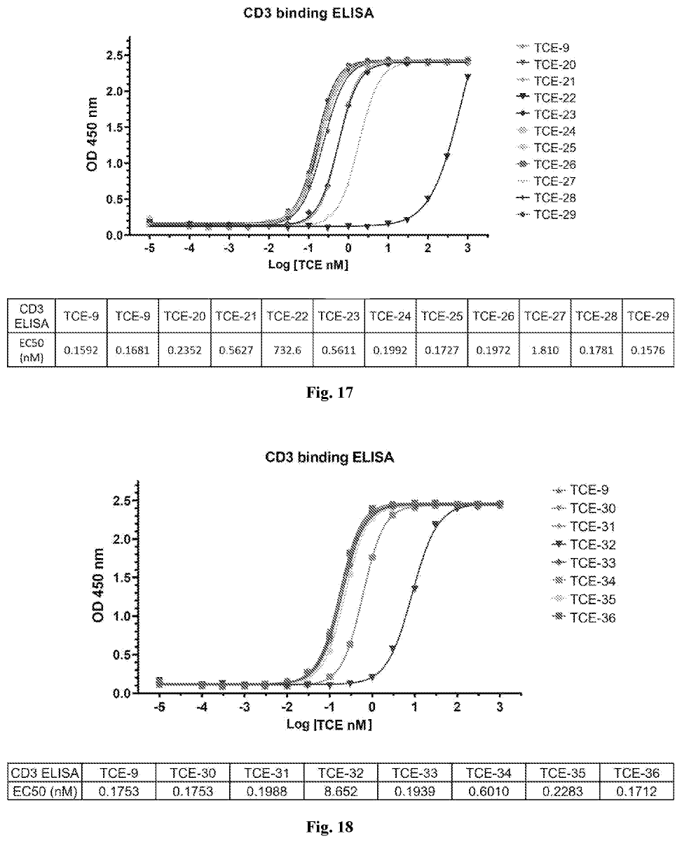

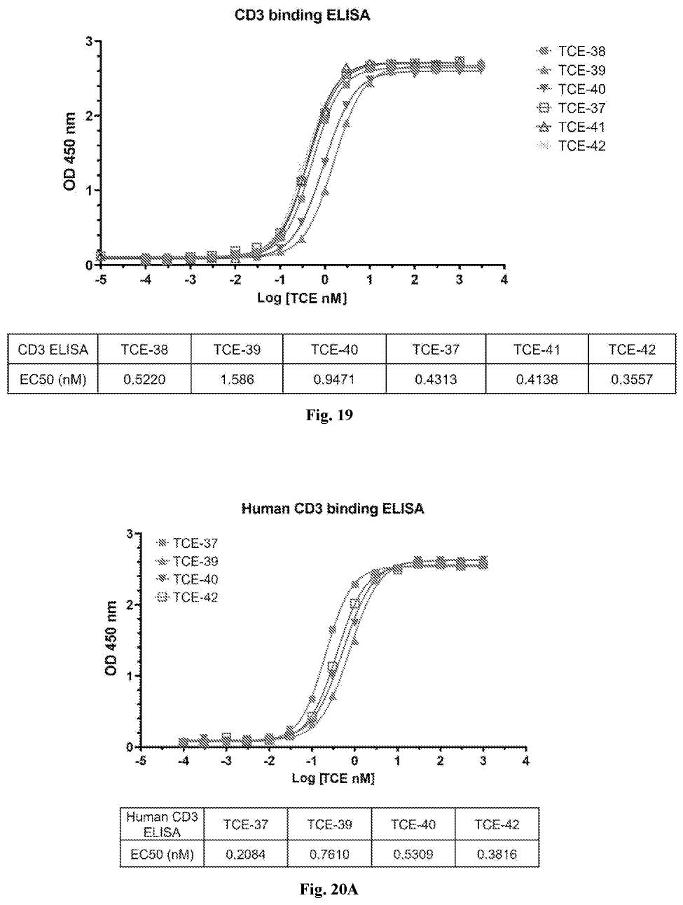

Binding curves for TCE-9 to TCE-19 binding to human CD3ε are shown in FIG. 16 along with calculated EC₅₀s. Binding curves for TCE-9 and TCE-20 to TCE-29 binding to human CD36 are shown in FIG. 17 along with calculated EC₅₀s. Binding curves and EC₅₀s for TCE-9 and TCE-30 to TCE-36 binding to human CD3ε are shown in FIG. 18. FIG. 19 shows binding curves and EC₅₀s for binding of TCE-37 to TCE-42 binding to CD3ε. FIGS. 20A-20B show binding curves and EC₅₀s for binding of TCE-37, TCE-39, TCE-40, and TCE-42 to human CD3 (FIG. 20A) and cyno CD3 (FIG. 20B) as measured by ELISA.

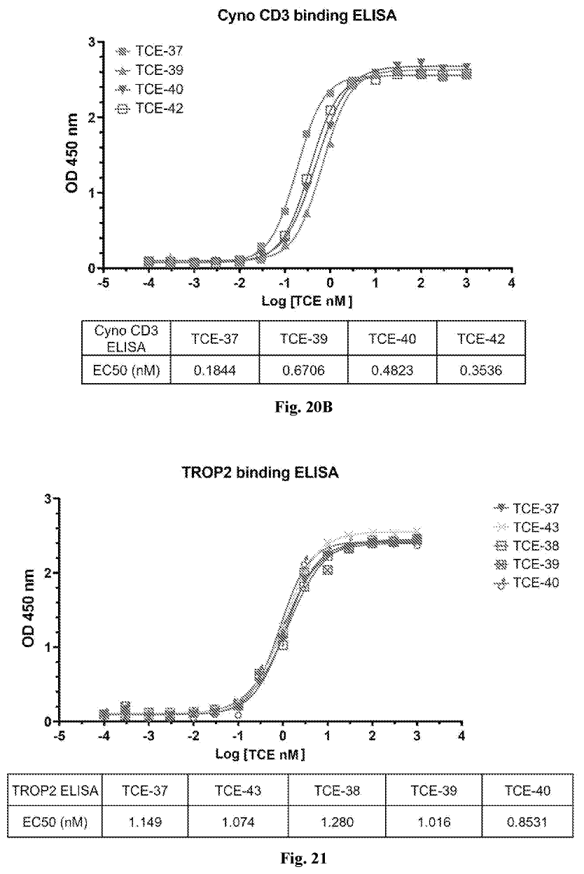

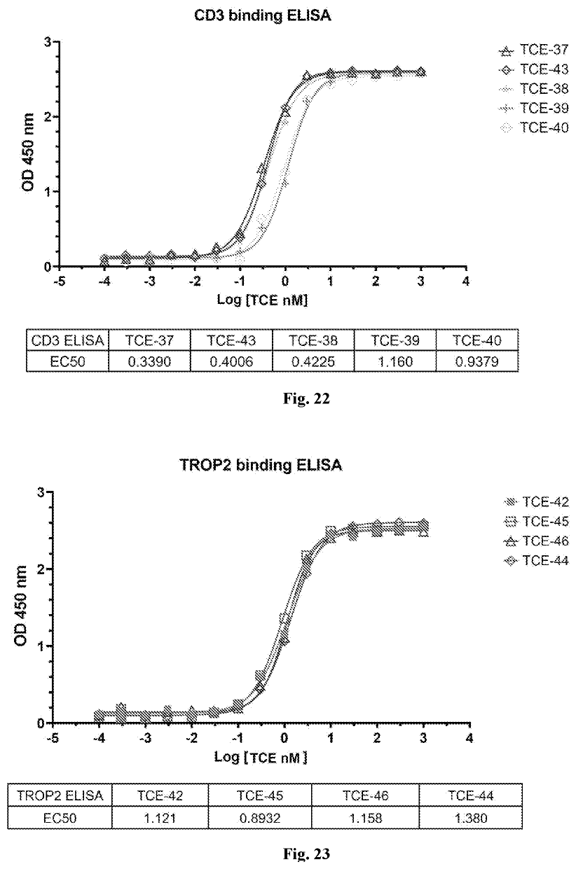

Binding curves and EC₅₀s for TROP2 binding by TCE-37, TCE-43, TCE-38, TCE-39, and TCE-40 as measured by ELISA are shown in FIG. 21. Binding curves and EC₅₀s for CD3 binding by TCE-37, TCE-43, TCE-38, TCE-39, and TCE-40 as measured by ELISA are shown in FIG. 22. Binding curves and EC₅₀s for TROP2 binding by TCE-42, TCE-45, TCE-46, and TCE-44 as measured by ELISA are shown in FIG. 23. Binding curves for CD3 binding by TCE-42, TCE-45, TCE-46, and TCE-44 as measured by ELISA are shown in FIG. 24.

### Kinetic Binding to CD3ε and TROP2 Via BLI

The wild-type (non-mutated) and alanine mutated TCEs were evaluated for their ability to bind CD36 or TROP2 via kinetic binding measurements using biolayer interferometry (BLI). Briefly, biotinylated CD3ε or TROP2 was loaded onto a streptavidin coated Octet® SAX biosensor, quenched in biocytin, and baselined in buffer. The TCEs diluted in buffer were then associated onto the antigen loaded biosensors. Sensors were then transferred to buffer where the TCEs were then dissociated from the sensors. Association and dissociation rates were measured in real time using an Octet® instrument. Exemplary experimental conditions and steps used for the kinetic binding measurements with CD3ε are shown in Table 16.

TABLE 16

| Step | Time |
|---|---|
| Baseline: Octet buffer | 60 seconds |
| Loading: 10 nM human | 300 seconds |
| CD3ε-biotin | |
| Biocytin quench | 300 seconds |
| (100 μM) | |
| Baseline: Octet buffer | 300 seconds |
| Association: 50 nM TCE | 300 seconds |
| Dissociation: Octet buffer | 600 seconds |

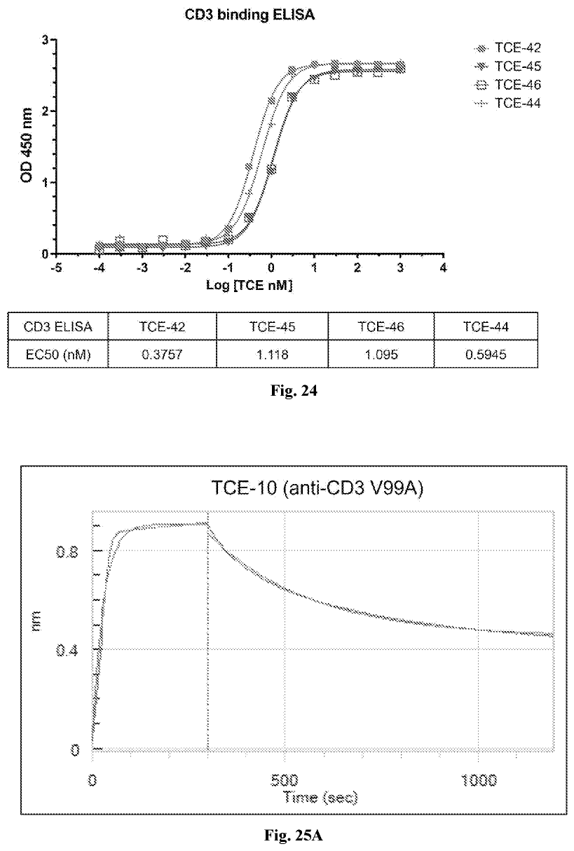

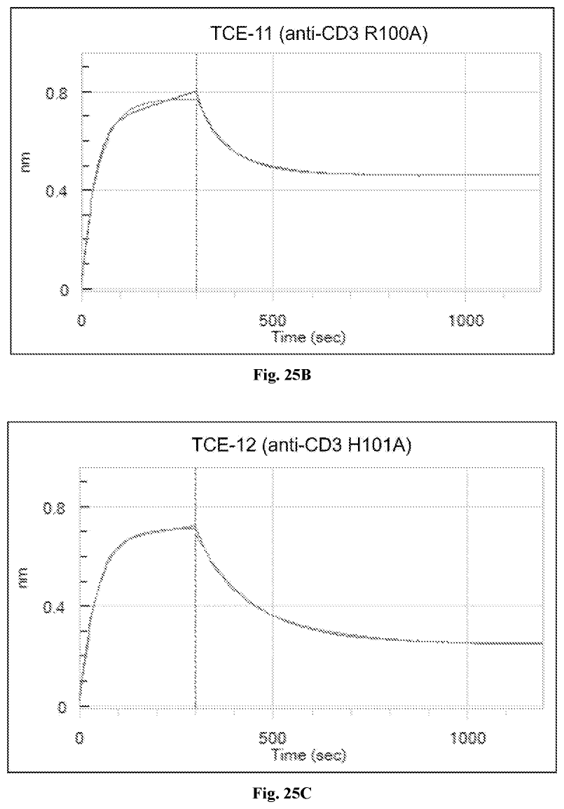

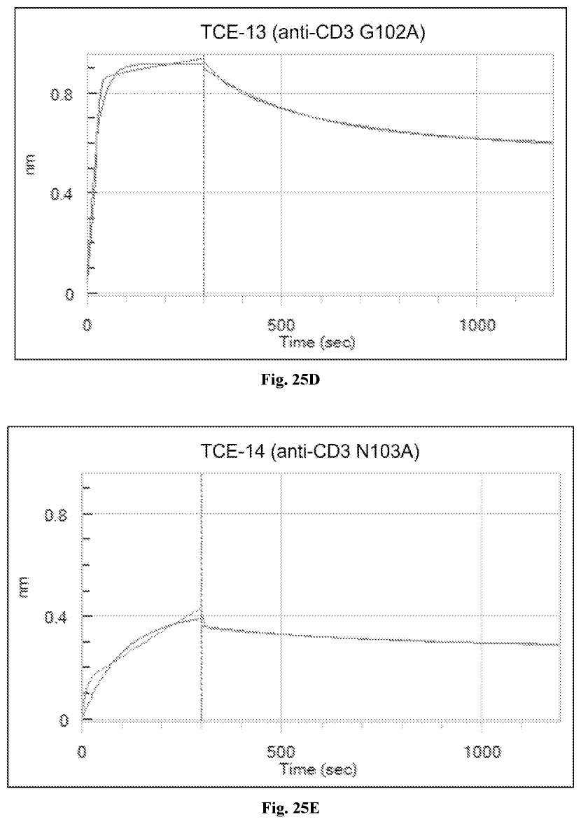

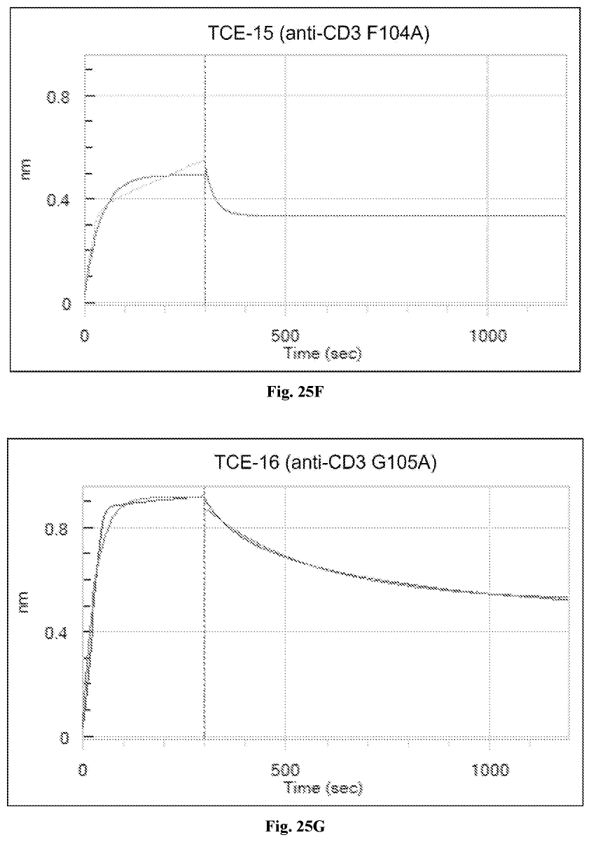

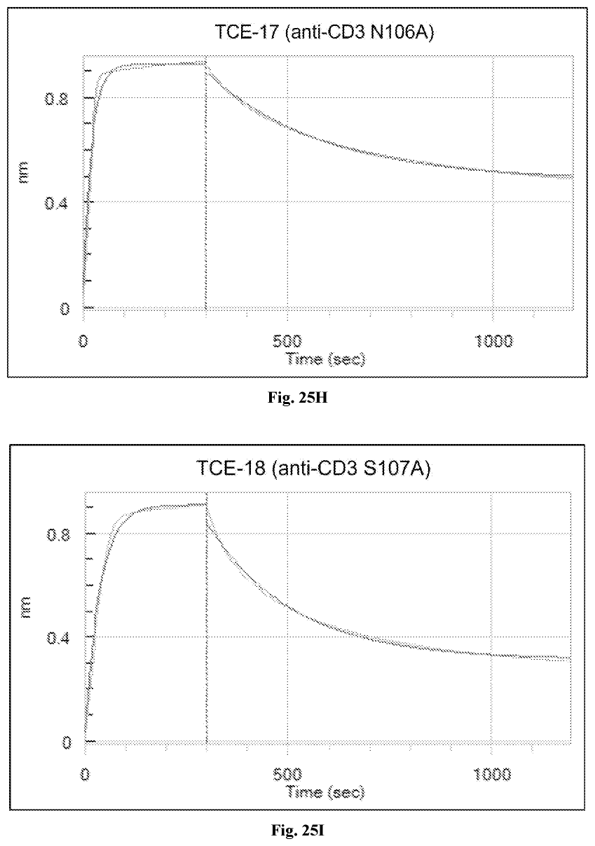

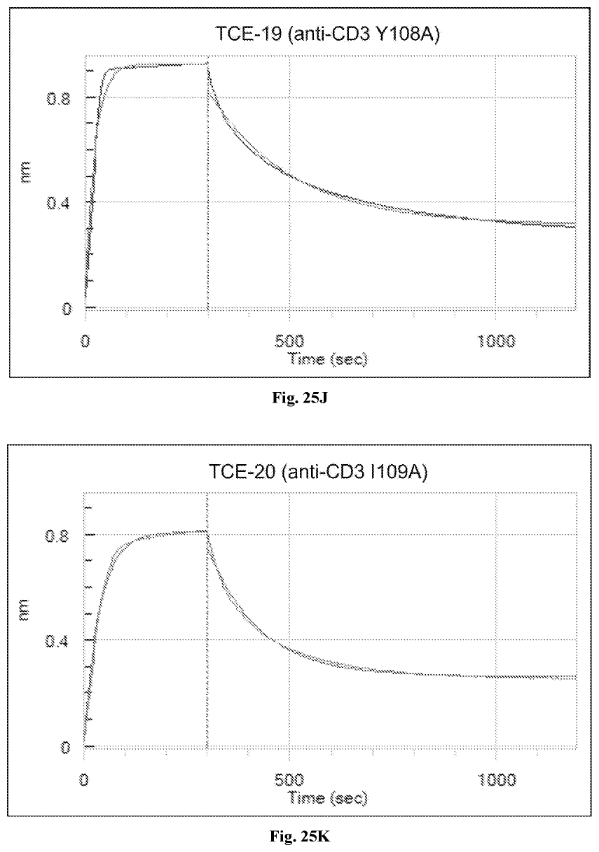

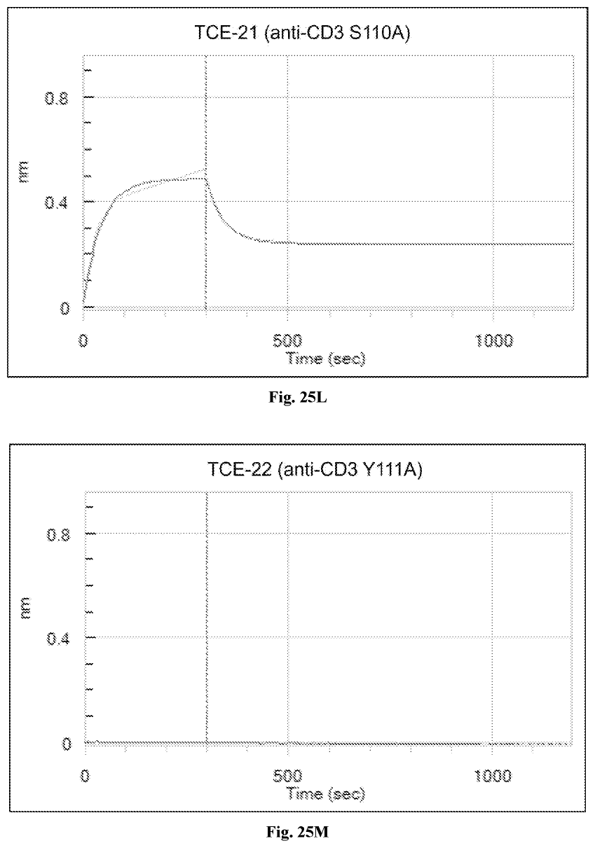

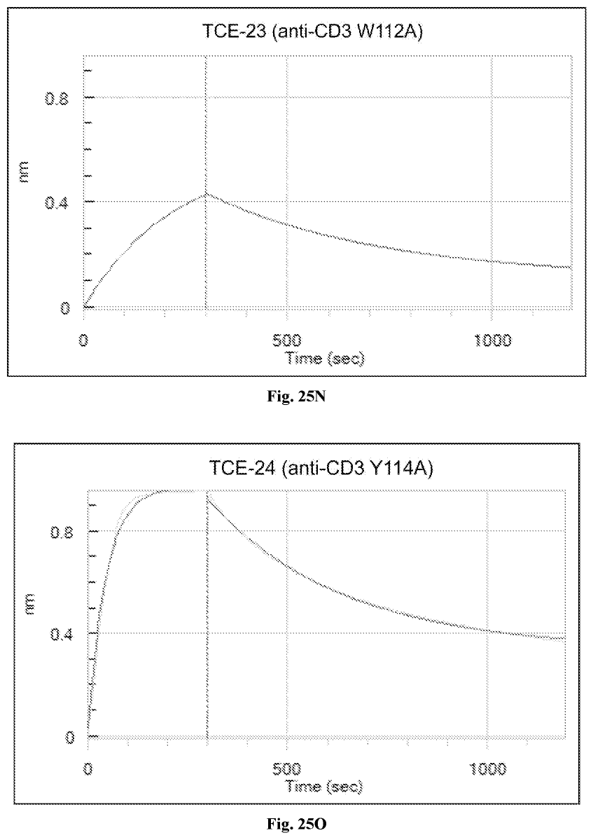

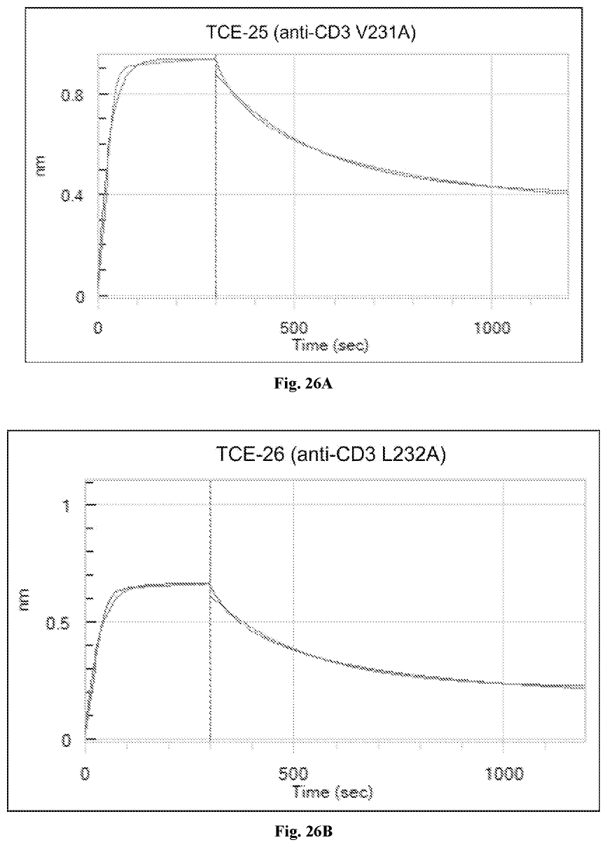

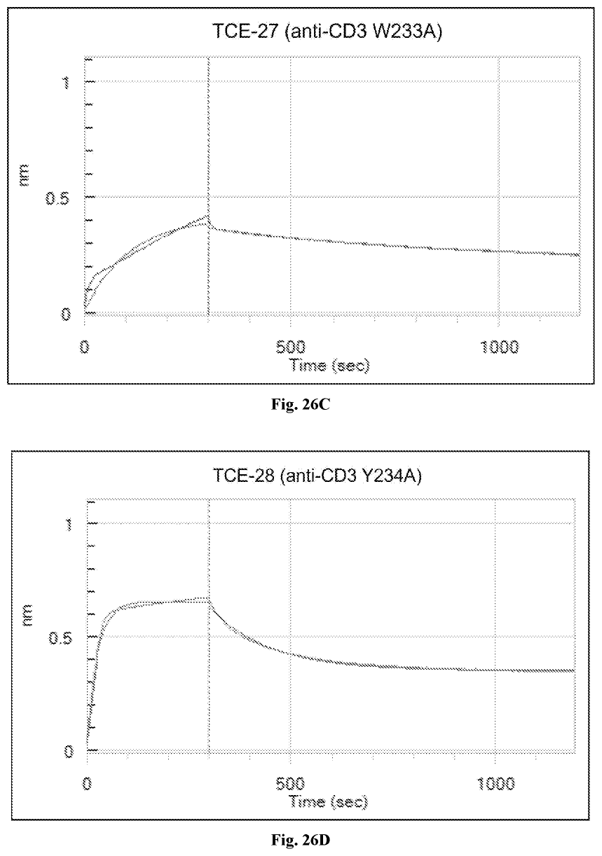

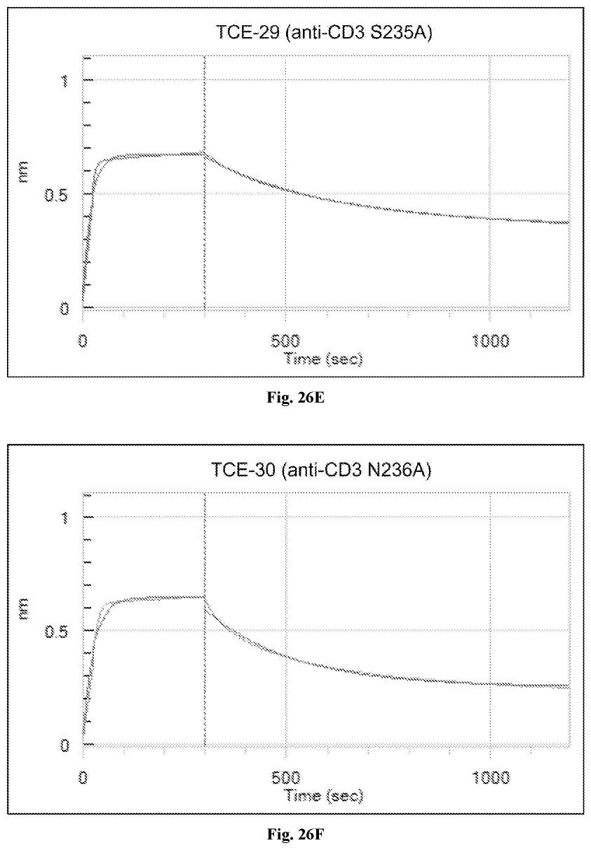

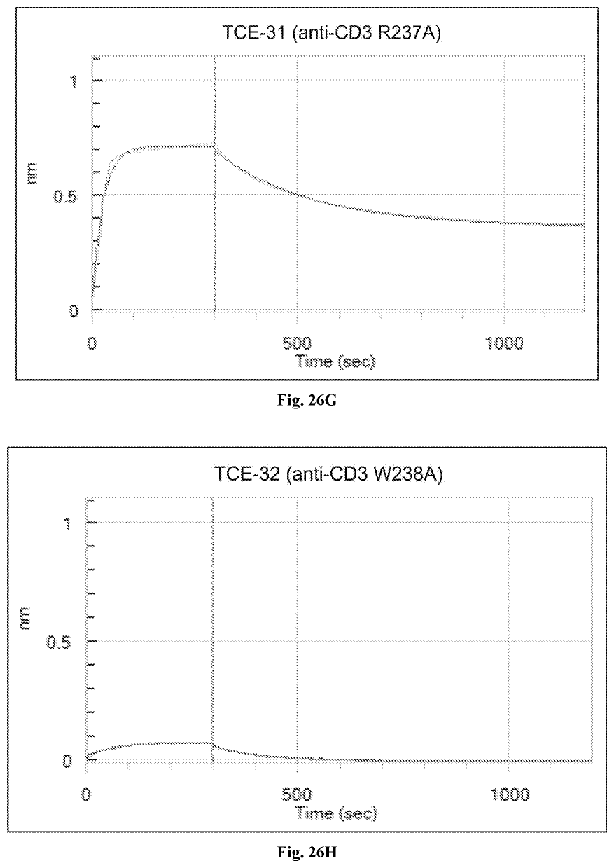

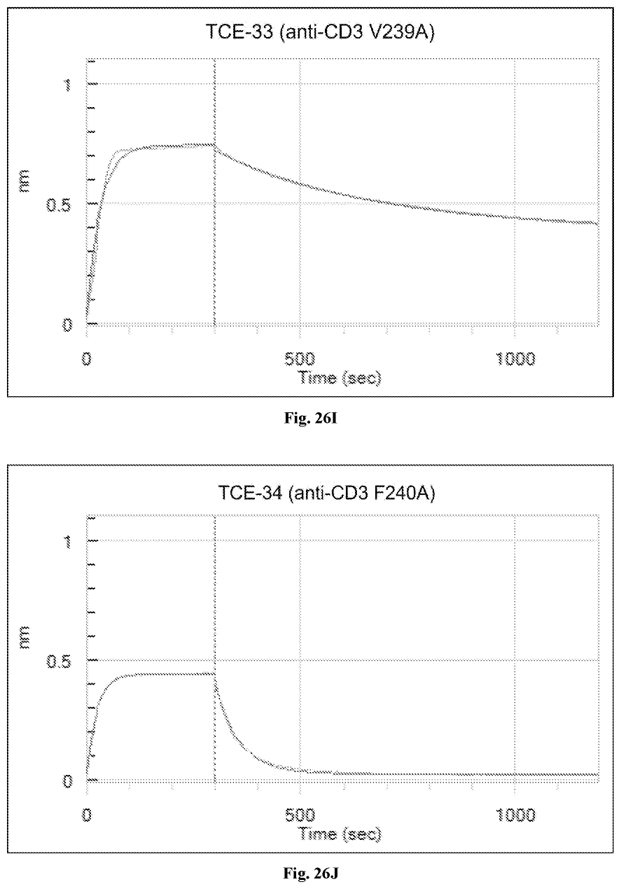

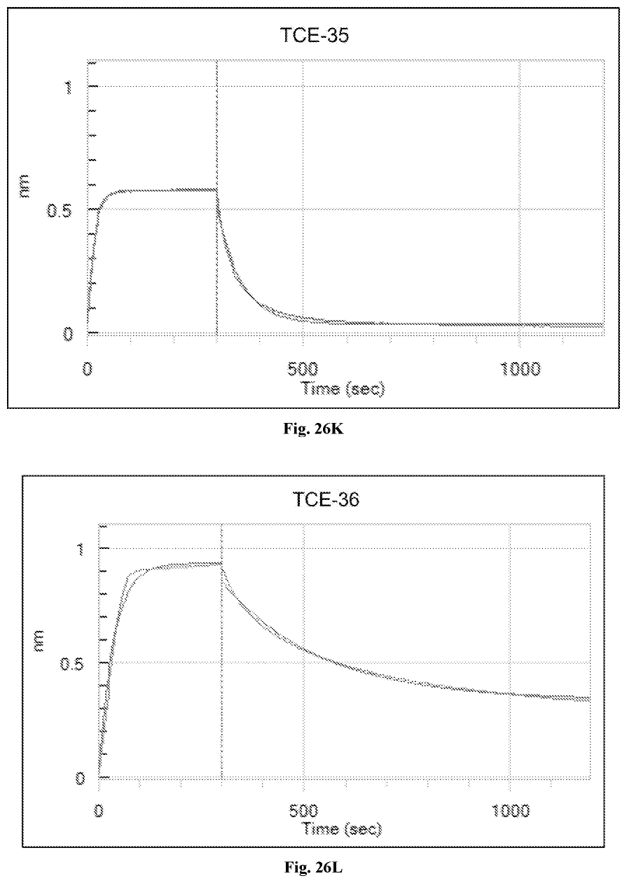

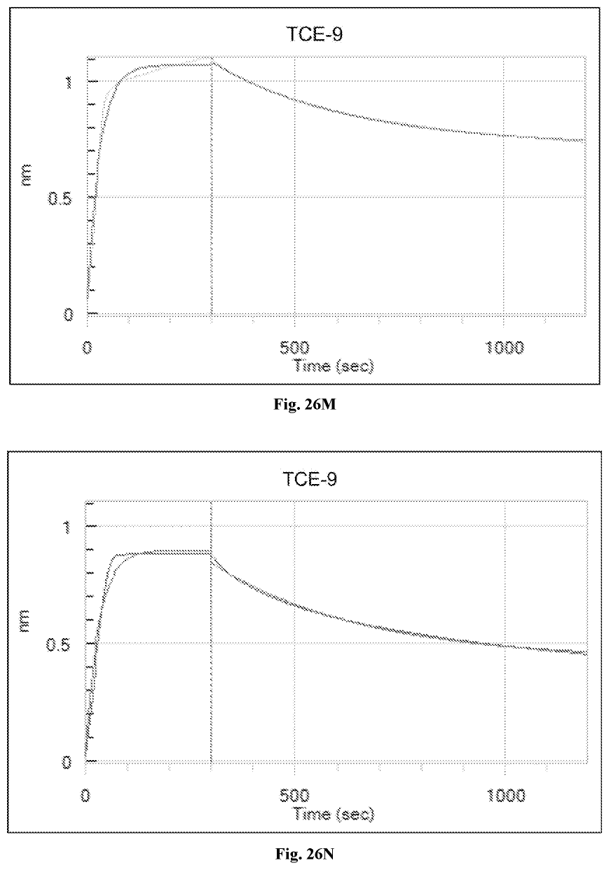

Example sensorgrams for TCE-10 to TCE-24 (anti-CD3 CDR3 heavy chain mutants) are shown in FIGS. 25A-25O. Example sensorgrams for TCE-25 to TCE-36 (including anti-CD3 CDR3 light chain mutants) and TCE-9 are shown in FIGS. 26A-26N. Calculated binding affinities (K_{D}s), association rates (kₒₙ), and dissociation rates (k_{dis}) for TCE-9 to TCE-36 from the BLI measurements are provided in Table 17.

TABLE 17

| Construct | | | | |
|---|---|---|---|---|
| description | K_{D} (M) | kₒₙ (1/M · s) | k_{dis} (1/s) | Response |
| TCE-10 | 6.06E−09 | 6.18E+05 | 3.75E−03 | 0.9106 |
| TCE-11 | 5.76E−08 | 2.08E+05 | 1.20E−02 | 0.7986 |
| TCE-12 | 2.37E−08 | 2.91E+05 | 6.90E−03 | 0.7208 |
| TCE-13 | 4.89E−09 | 7.52E+05 | 3.68E−03 | 0.9357 |
| TCE-14 | 1.68E−08 | 1.45E+05 | 2.43E−03 | 0.425 |
| TCE-15 | | | 4.42E−02 | 0.5478 |
| TCE-16 | 6.43E−09 | 5.72E+05 | 3.67E−03 | 0.9196 |
| TCE-17 | 3.83E−09 | 9.22E+05 | 3.53E−03 | 0.9385 |
| TCE-18 | 1.07E−08 | 4.47E+05 | 4.76E−03 | 0.909 |
| TCE-19 | 6.63E−09 | 7.48E+05 | 4.96E−03 | 0.9291 |
| TCE-20 | 2.25E−08 | 3.56E+05 | 8.00E−03 | 0.8159 |
| TCE-21 | | | 2.38E−02 | 0.5216 |
| TCE-22 | | | | −0.0009 |
| TCE-23 | 5.00E−08 | 4.65E+04 | 2.32E−03 | 0.4218 |
| TCE-24 | 7.86E−09 | 3.77E+05 | 2.97E−03 | 0.9557 |
| TCE-25 | 6.26E−09 | 6.22E+05 | 3.90E−03 | 0.939 |
| TCE-26 | 8.39E−09 | 5.23E+05 | 4.38E−03 | 0.6629 |
| TCE-27 | 1.23E−08 | 1.46E+05 | 1.79E−03 | 0.4142 |
| TCE-28 | 1.11E−08 | 6.26E+05 | 6.94E−03 | 0.6737 |
| TCE-29 | 3.41E−09 | 9.30E+05 | 3.17E−03 | 0.6816 |
| TCE-30 | 7.03E−09 | 6.72E+05 | 4.72E−03 | 0.6504 |
| TCE-31 | 6.51E−09 | 6.64E+05 | 4.32E−03 | 0.7284 |
| TCE-32 | 3.10E−08 | 2.50E+05 | 7.75E−03 | 0.0683 |
| TCE-33 | 4.76E−09 | 5.70E+05 | 2.71E−03 | 0.7422 |
| TCE-34 | 3.61E−08 | 4.90E+05 | 1.77E−02 | 0.444 |
| TCE-35 | 2.10E−08 | 8.62E+05 | 1.81E−02 | 0.5832 |
| TCE-36 | 9.11E−09 | 4.63E+05 | 4.22E−03 | 0.9306 |
| TCE-9 | 4.91E−09 | 6.08E+05 | 2.98E−03 | 1.1051 |

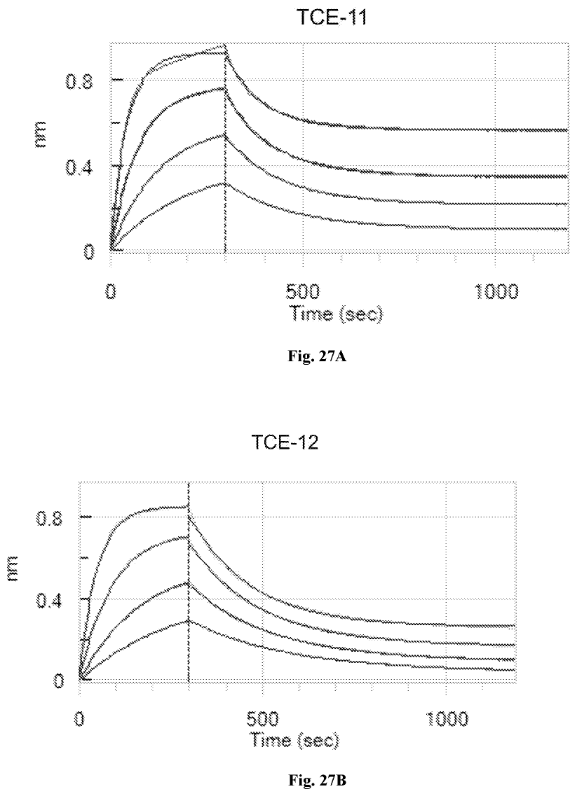

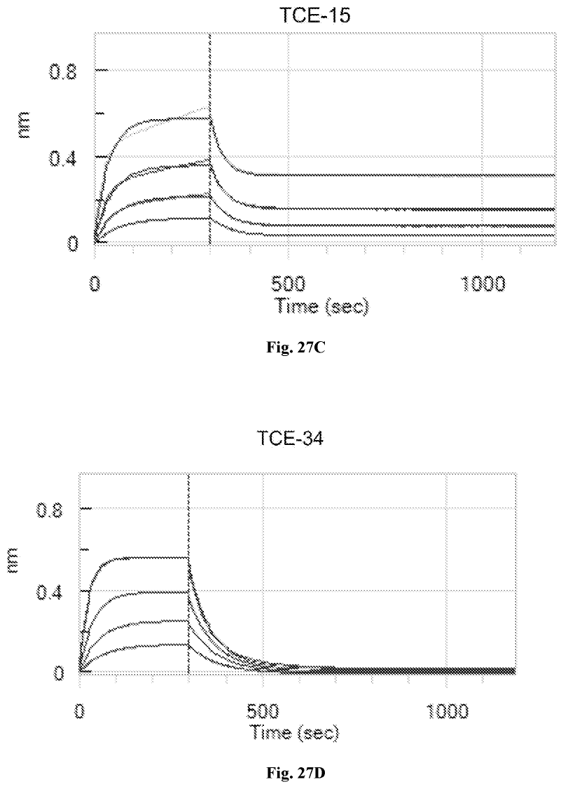

FIGS. 27A-27D show example BLI sensorgrams for TCE-11 (FIG. 27A, anti-CD3 R100A mutant), TCE-12 (FIG. 27B, anti-CD3 H101A mutant), TCE-15 (FIG. 27C, anti-CD3 F104A mutant), and TCE-34 (FIG. 27D, anti-CD3 F240A mutant) binding to CD38 using the experimental conditions and steps of Table 18. Calculated K_{D}s and kₒₙ and k_{dis} rates for TCE-11, TCE-12, TCE-15, and TCE-34 binding to CD38 are provided in Table 19.

TABLE 18

| Step | Time |
|---|---|
| Sensor: SAX | — |
| Baseline: Octet buffer | 60 seconds |
| Loading: 10 nM human | 300 seconds |
| CD3ε-biotin | |
| Biocytin quench | 300 seconds |
| (100 μM) | |
| Baseline: Octet buffer | 300 seconds |
| Association: | 300 seconds |
| 50 nM TCE | |
| 25 nM TCE | |
| 12.5 nM TCE | |
| 6.25 nM TCE | |
| Dissociation: Octet buffer | 900 seconds |

TABLE 19

| Construct | Loading | | | |
|---|---|---|---|---|
| description | Sample ID | K_{D} (M) | Kₒₙ (1/M · s) | k_{dis} (1/s) |
| TCE-11 | CD3e-bio [10 nM] | 4.42E−08 | 2.42E+05 | 1.07E−02 |
| TCE-12 | CD3e-bio [10 nM] | 2.09E−08 | 2.84E+05 | 5.93E−03 |
| TCE-15 | CD3e-bio [10 nM] | | | 3.36E−02 |
| TCE-34 | CD3e-bio [10 nM] | 3.46E−08 | 4.55E+05 | 1.57E−02 |

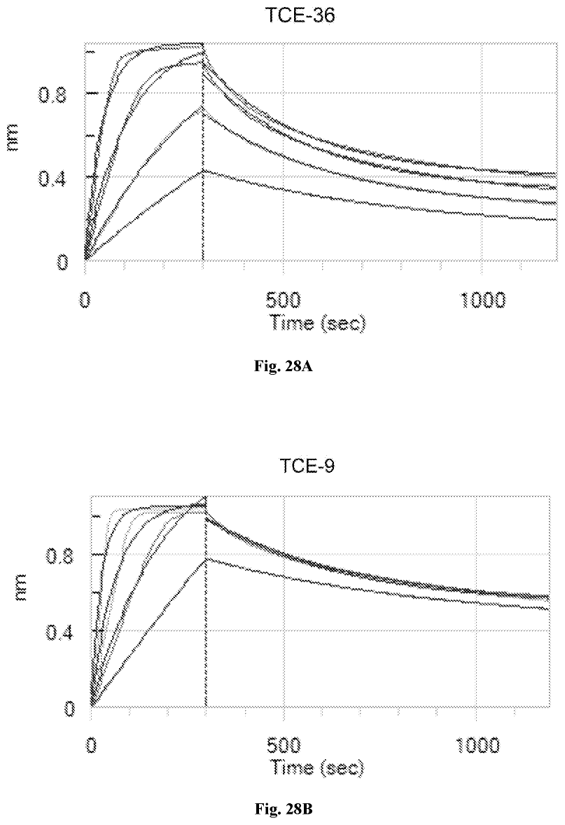

FIGS. 28A-28B show example BLI sensorgrams for CD3& binding by wild-type constructs TCE-36 (FIG. 28A) and TCE-9 (FIG. 28B) under the experimental conditions and steps of Table 18. Calculated K_{D}s and kₒₙ and k_{dis} rates for TCE-36 and TCE-9 binding to CD3& are provided in Table 20.

TABLE 20

| Construct | Loading | | | |
|---|---|---|---|---|
| description | Sample ID | K_{D} (M) | Kₒₙ (1/M · s) | K_{dis} (1/s) |
| TCE-36 | CD3e-bio [10 nM] | 1.07E−08 | 3.60E+05 | 3.85E−03 |
| TCE-9 | CD3e-bio [10 nM] | 5.30E−09 | 5.11E+05 | 2.71E−03 |

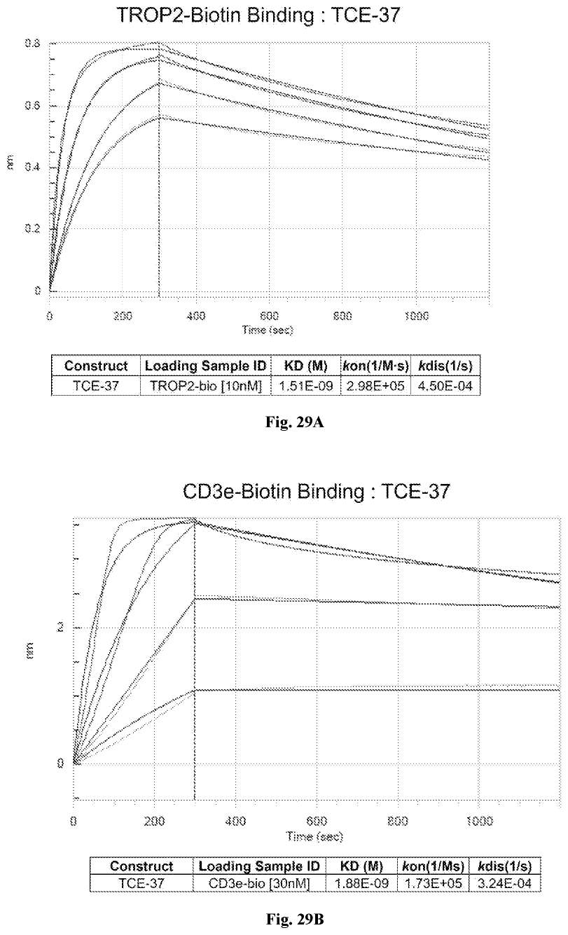

Example sensorgrams for TCE-37 (wild-type anti-CD3, anti-TROP2 F108A) binding to TROP2 and CD3ε as well as calculated K_{D}s, kₒₙ, and k_{dis} are shown in FIGS. 29A-29B. Exemplary experimental conditions and steps used for the measurements of FIGS. 29A-29B are provided in Table 21.

TABLE 21

| Step | Time |
|---|---|
| Sensor: SAX | — |
| Baseline: Octet buffer | 60 seconds |
| Loading: | 300 seconds |
| 10 nM TROP2-biotin | |
| 30 nM CD3e-biotin | |
| Biocytin quench | 300 seconds |
| (100 μM) | |
| Baseline: Octet buffer | 300 seconds |
| Association: | 300 seconds |
| 100 nM TCE-37 | |
| 50 nM TCE-37 | |
| 25 nM TCE-37 | |
| 12.5 nM TCE-37 | |
| Dissociation: Octet buffer | 900 seconds |

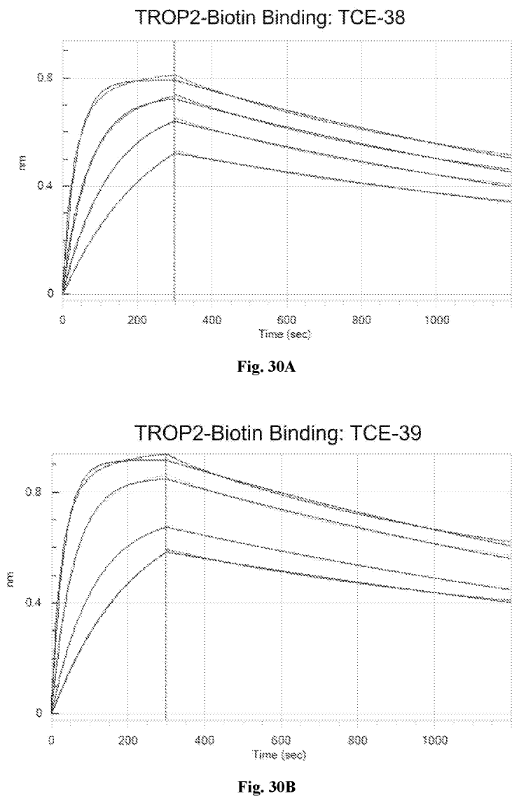

FIGS. 30A-30B show exemplary sensorgrams for binding of double mutant TCE-38 (anti-CD3 H101A, anti-TROP2 F108A) and double mutant TCE-39 (anti-CD3 F104A, anti-TROP2 F108A) to TROP2 using the experimental conditions and steps of Table 22. Calculated K_{D}s and kₒₙ and k_{dis} rates for binding of TCE-38 and TCE-39 to TROP2 are provided in Table 23.

TABLE 22

| Step | Time |
|---|---|
| Sensor: SAX | — |
| Baseline: Octet buffer | 60 seconds |
| Loading: | 300 seconds |
| 10 nM TROP2-biotin | |
| 30 nM CD3e-biotin | |
| Biocytin quench | 300 seconds |
| (100 μM) | |
| Baseline: Octet buffer | 300 seconds |
| Association: | 300 seconds |
| 100 nM TCE-38 | |
| 50 nM TCE-38 | |
| 25 nM TCE-38 | |
| 12.5 nM TCE-38 | |
| 100 nM TCE-39 | |
| 50 nM TCE-39 | |
| 25 nM TCE-39 | |
| 12.5 nM TCE-39 | |
| Dissociation: Octet buffer | 900 seconds |

TABLE 23

| Construct | Loading | | | |
|---|---|---|---|---|
| description | Sample ID | K_{D} (M) | kₒₙ (1/M · s) | k_{dis} (1/s) |
| TCE-38 | TROP2-bio [10 nM] | 1.56E−09 | 3.04E+05 | 4.75E−04 |
| TCE-39 | TROP2-bio [10 nM] | 1.21E−09 | 3.42E+05 | 4.14E−04 |

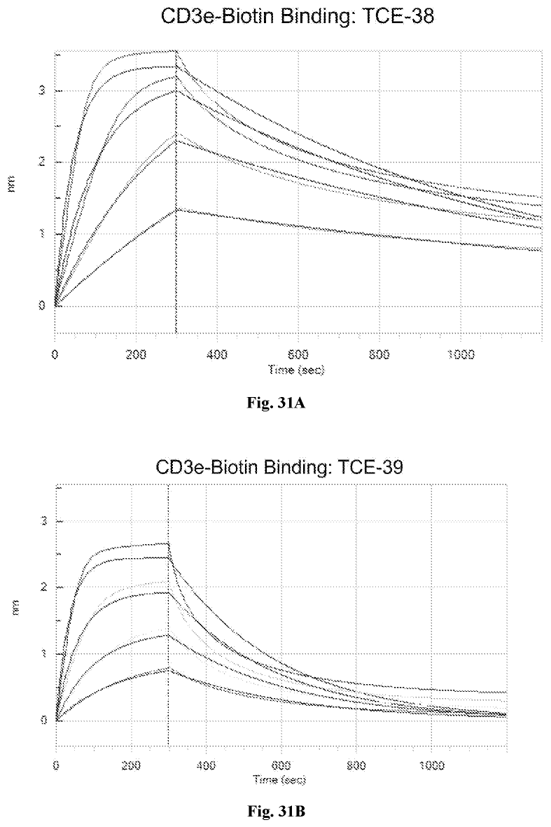

FIGS. 31A-31B show exemplary sensorgrams for binding of double mutant TCE-38 (anti-CD3 H101A, anti-TROP2 F108A) and double mutant TCE-39 (anti-CD3 F104A, anti-TROP2 F108A) to CD36 using the experimental conditions and steps of Table 22. Calculated K_{D}s and kₒₙ and k_{dis} rates for binding of TCE-38 and TCE-39 to CD3ε are provided in Table 24.

TABLE 24

| Construct | Loading | | | |
|---|---|---|---|---|
| description | Sample ID | K_{D} (M) | kₒₙ (1/M · s) | k_{dis} (1/s) |
| TCE-38 | CD3e-bio [30 nM] | 5.43E−09 | 2.04E+05 | 1.11E−03 |
| TCE-39 | CD3e-bio [30 nM] | 1.40E−08 | 2.47E+05 | 3.46E−03 |

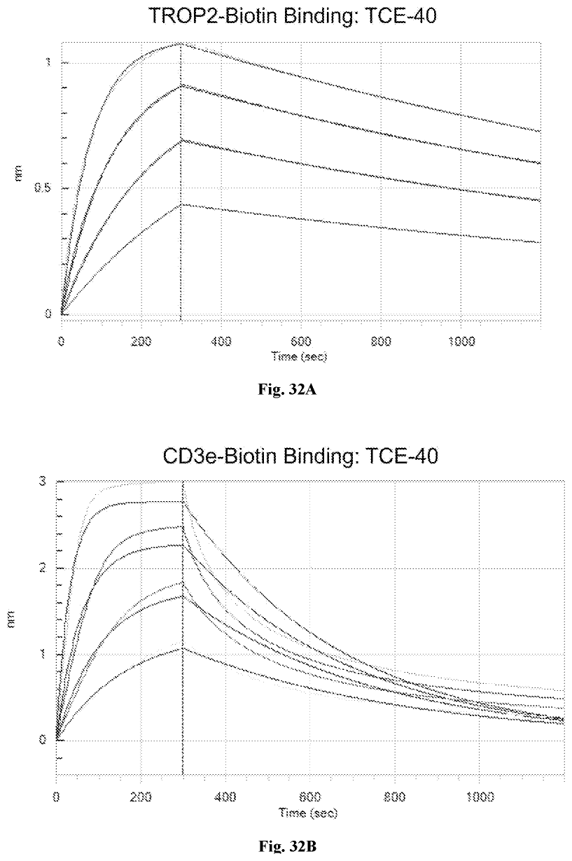

Example sensorgrams for double mutant TCE-40 (anti-CD3 F240A, anti-TROP2 F108A) binding to TROP2 and CD3ε are shown in FIGS. 32A-32B. Experimental conditions and steps used for the measurements are provided in Table 25. Calculated K_{D}s and kₒₙ and k_{dis} rates for binding of TCE-40 to TROP2 and CD3ε are provided in Table 26.

TABLE 25

| Step | Time |
|---|---|
| Sensor: SAX | — |
| Baseline: Octet buffer | 60 seconds |
| Loading: | 300 seconds |
| 10 nM TROP2-biotin | |
| 30 nM CD3e-biotin | |
| Biocytin quench | 300 seconds |
| (100 μM) | |
| Baseline: Octet buffer | 300 seconds |
| Association: | 300 seconds |
| 100 nM TCE-40 | |
| 50 nM TCE-40 | |
| 25 nM TCE-40 | |
| 12.5 nM TCE-40 | |
| Dissociation: Octet buffer | 900 seconds |

TABLE 26

| Construct | Loading | | | | |
|---|---|---|---|---|---|
| description | Sample ID | | K_{D} (M) | Kₒₙ (1/M · s) | k_{dis} (1/s) |
| TCE-40 | TROP2-bio | [10 nM] | 2.92E−09 | 1.62E+05 | 4.73E−04 |
| TCE-40 | CD3e-bio | [30 nM] | 9.12E−09 | 2.90E+05 | 2.64E−03 |

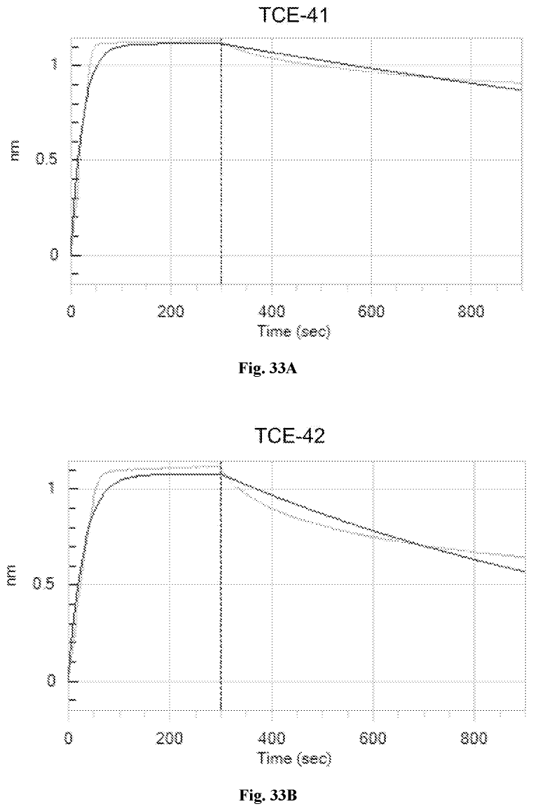

Exemplary sensorgrams for TCE-41 (anti-TROP2 F108A mutant) and TCE-42 (anti-TROP2 F108A mutant) binding to CD3ε are shown in FIGS. 33A-33B. Exemplary experimental conditions and steps used for the measurements of FIGS. 33A-33B are provided in Table 27, and calculated K_{D}s and kₒₙ and k_{dis} rates for TCE-41 and TCE-42 binding to CD3ε are provided in Table 28.

TABLE 27

| Step | Time |
|---|---|
| Baseline: Octet buffer | 60 seconds |
| Loading: | 300 seconds |
| 10 nM human | |
| CD3-biotin | |
| Biocytin quench | 300 seconds |
| (100 μM) | |
| Baseline: Octet buffer | 300 seconds |
| Association: | 300 seconds |
| 50 nM TCE | |
| Dissociation: Octet buffer | 900 seconds |

TABLE 28

| Construct | Loading | | | |
|---|---|---|---|---|
| description | Sample ID | K_{D} (M) | kₒₙ (1/M · s) | k_{dis} (1/s) |
| TCE-41 | Biotinylated CD3e | 4.67E−10 | 8.85E+05 | 4.14E−04 |
| TCE-42 | Biotinylated CD3e | 1.62E−09 | 6.59E+05 | 1.07E−03 |

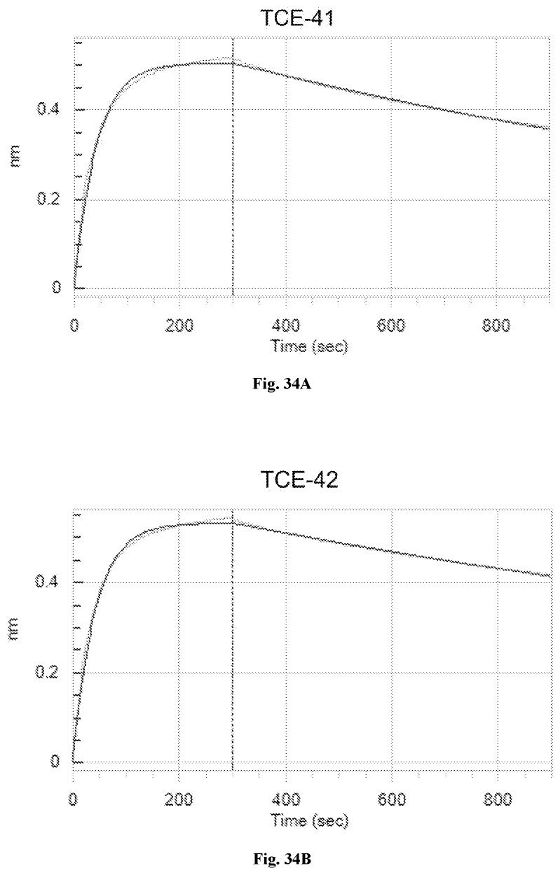

Exemplary sensorgrams for TCE-41 (anti-TROP2 F108A mutant) and TCE-42 (anti-TROP2 F108A mutant) binding to TROP2 are shown in FIGS. 34A-34B. Exemplary experimental conditions and steps used for the measurements of FIGS. 34A-34B are provided in Table 29, and calculated K_{D}s and kₒₙ and k_{dis} rates for TCE-41 and TCE-42 binding to TROP2 are provided in Table 30.

TABLE 29

| Step | Time |
|---|---|
| Baseline: Octet buffer | 60 seconds |
| Loading: | 300 seconds |
| 10 nM human TROP2- | |
| biotin | |
| Biocytin quench | 300 seconds |
| (100 μM) | |
| Baseline: Octet buffer | 300 seconds |
| Association: | 300 seconds |
| 50 nM TCE | |
| Dissociation: Octet buffer | 900 seconds |

TABLE 30

| Construct | | | | |
|---|---|---|---|---|
| description | Loading Sample ID | K_{D} (M) | kₒₙ (1/M · s) | k_{dis} (1/s) |
| TCE-41 | Biotinylated TROP2 | 1.22E−09 | 4.74E+05 | 5.80E−04 |
| TCE-42 | Biotinylated TROP2 | 8.81E−10 | 4.76E+05 | 4.19E−04 |

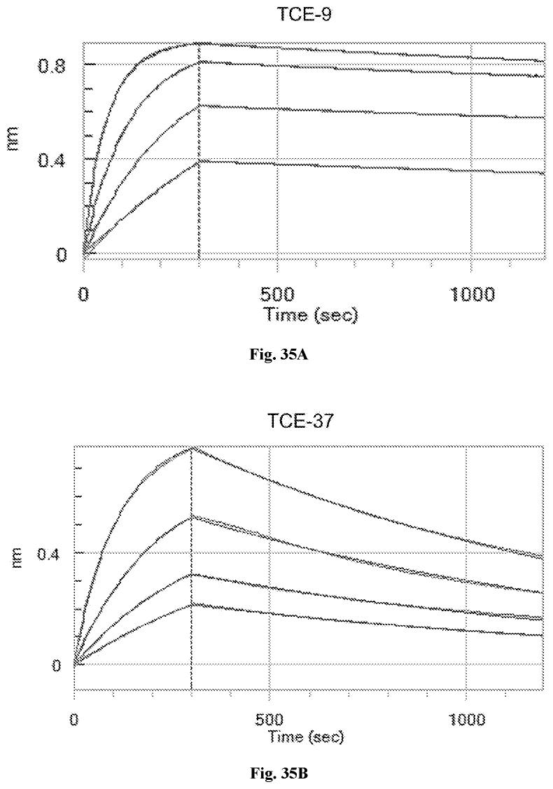

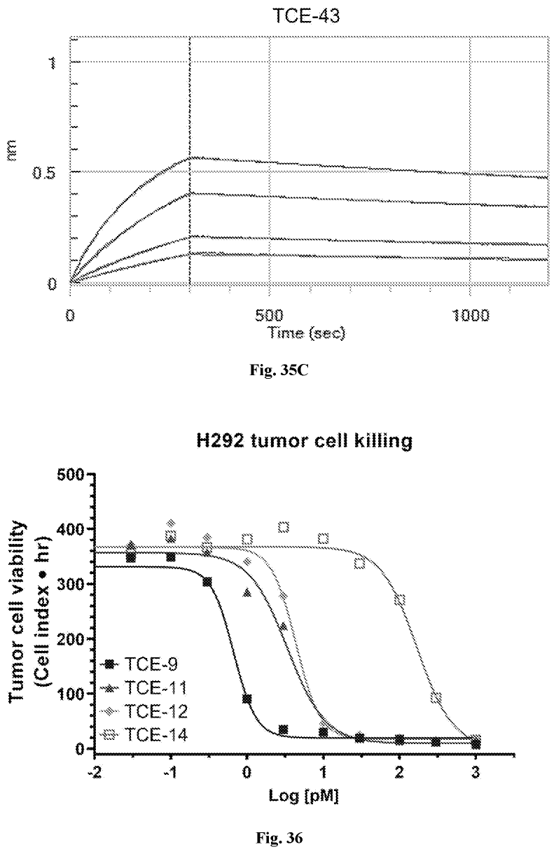

Exemplary sensorgrams for TCE-9 (wild-type anti-CD3, wild-type anti-TROP2), TCE-37 (anti-TROP2 F108A mutant), and TCE-43 (anti-TROP2 D109A mutant) binding to TROP2 are shown in FIGS. 35A-35C. Exemplary experimental conditions and steps used for the measurements of FIGS. 35A-35C are provided in Table 31, and calculated K_{D}s and kₒₙ and k_{dis} rates are provided in Table 32.

TABLE 31

| Step | Time |
|---|---|
| Sensor: SAX | — |
| Baseline: Octet buffer | 60 seconds |
| Loading: Human TROP2- | 300 seconds |
| biotin [10 nM] | |
| Biocytin quench (100 | 300 seconds |
| μM) | |
| Baseline: Octet buffer | 300 seconds |
| Association: | 300 seconds |
| 50 nM, 25 nM, | |
| 12.5 nM, 6.25 nM | |
| TCE | |
| Dissociation: Octet buffer | 600 seconds |

TABLE 32

| Construct | Loading | | K_{D} | | |
|---|---|---|---|---|---|
| description | Sample ID | K_{D} (M) | Error | kₒₙ (1/M · s) | k_{dis} (1/s) |
| TCE-37 | hTROP2- | 4.94E−09 | 1.28E−11 | 1.62E+05 | 7.97E−04 |
| | Biotin | | | | |
| TCE-43 | hTROP2- | 1.91E−09 | 8.06E−12 | 1.04E+05 | 1.99E−04 |
| | Biotin | | | | |
| TCE-9 | hTROP2- | 2.60E−10 | 1.72E−12 | 3.79E+05 | 9.85E−05 |
| | Biotin | | | | |

### Example 8: Sequence Activity Relationships

Data summarizing CD38 binding parameters obtained from the above equilibrium and kinetic binding studies are provided in Table 33 (anti-CD3 CDR3 LC alanine mutated constructs) and Tables 34-35 (anti-CD3 CDR3 HC alanine mutated constructs). The tables also classify each alanine mutation in the CDR3 LC and CDR3 HC regions as tolerated (T), moderately tolerated (MT), or not tolerated (NT). Alanine mutations at residues which resulted in significantly weakened binding relative to the non-mutated TCE construct TCE-9 were characterized as NT, and those residues are considered key residues for CD3ε binding. Alanine mutations at residues which showed comparable binding parameters to TCE-9 were characterized as T and are considered non-critical for CD3ε binding. Alanine mutations which at residues resulted in somewhat weakened binding relative to TCE-9 were characterized as MT.

As can be seen from Table 33, alanine mutations at residues W233 and W238 of the anti-CD3 CDR3 LC were not tolerated indicating that these residues are important for binding to CD3. Additionally, Tables 34-35 demonstrate that alanine mutations at N103, F104, Y 111, and W 112 of the anti-CD3 CDR3 HC were not tolerated indicating that these residues are also important for binding to CD3.

TABLE 33
CD3ε Binding and SAR Data for anti-CD3 CDR3 LC Alanine Scanning Mutants

| | TCE9, | | | | | | | | | | |
|---|---|---|---|---|---|---|---|---|---|---|---|
| Construct | wt | TCE25 | TCE26 | TCE27 | TCE28 | TCE29 | TCE30 | TCE31 | TCE32 | TCE33 | TCE34 |
| CD3 K_{D} (nM) | 4.91 | 6.26 | 8.39 | 12.30 | 11.10 | 3.41 | 7.03 | 6.51 | 31.00 | 4.76 | 36.10 |
| CD3 t_{1/2} (min) | 3.88 | 2.96 | 2.64 | 6.45 | 1.66 | 3.64 | 2.45 | 2.67 | 1.49 | 4.26 | 0.65 |
| CD3 Octet response | 1.11 | 0.94 | 0.66 | 0.41 | 0.67 | 0.68 | 0.65 | 0.73 | 0.07 | 0.74 | 0.44 |
| CD3 EC₅₀ ELISA (nM) | 0.17 | 0.17 | 0.20 | 1.81 | 0.18 | 0.16 | 0.18 | 0.20 | 8.65 | 0.19 | 0.60 |
| Fold shift in EC₅₀ | — | 1 | 1.18 | 10.6 | 1.05 | 0.94 | 1.05 | 1.18 | 50.9 | 1.12 | 3.53 |
| relative to wt | | | | | | | | | | | |
| CDR3 LC Ala scan | — | V | L | W | Y | S | N | R | W | V | F |
| Amino acid position | — | 231 | 232 | 233 | 234 | 235 | 236 | 237 | 238 | 239 | 240 |
| Mutation tolerated? | — | T | T | NT | T | T | T | T | NT | T | MT |

TABLE 34
CD3ε Binding and SAR Data for anti-CD3 CDR3 HC Alanine Scanning Mutants

| | TCE9, | not | | | | | | | |
|---|---|---|---|---|---|---|---|---|---|
| Construct | wt | mutated | TCE10 | TCE11 | TCE12 | TCE13 | TCE14 | TCE15 | TCE16 |
| CD3 K_{D} (nM) | 4.91 | — | 6.1 | 57.6 | 23.7 | 4.9 | 16.8 | | 6.4 |
| CD3 t_{1/2} (min) | 3.88 | — | 3.1 | 1.0 | 1.7 | 3.1 | 4.8 | 0.3 | 3.1 |
| CD3 Octet response | 1.11 | — | 0.91 | 0.80 | 0.72 | 0.94 | 0.43 | 0.55 | 0.92 |
| CD3 EC₅₀ ELISA (nM) | 0.17 | — | 0.20 | 0.28 | 0.27 | 0.22 | 3.13 | 1.14 | 0.19 |
| Fold shift in EC₅₀ | — | — | 1.18 | 1.65 | 1.59 | 1.29 | 18.4 | 6.71 | 1.12 |
| relative to wt | | | | | | | | | |
| CDR3 HC Ala scan | — | A | V | R | H | G | N | F | G |
| Amino acid position | — | — | 99 | 100 | 101 | 102 | 103 | 104 | 105 |
| Mutation tolerated? | — | — | T | MT | MT | T | NT | NT | T |

TABLE 35
CD3ε Binding and SAR Data for anti-CD3 CDR3 HC Alanine Scanning Mutants

| | | | | | | | | not | |
|---|---|---|---|---|---|---|---|---|---|
| Construct | TCE17 | TCE18 | TCE19 | TCE20 | TCE21 | TCE22 | TCE23 | mutated | TCE24 |
| CD3 K_{D} (nM) | 3.8 | 10.7 | 6.6 | 22.5 | | | 50.0 | — | 7.9 |
| CD3 t_{1/2} (min) | 3.3 | 2.4 | 2.3 | 1.4 | 0.5 | | 5.0 | — | 3.9 |
| CD3 Octet response | 0.94 | 0.91 | 0.93 | 0.82 | 0.52 | 0.00 | 0.42 | — | 0.96 |
| CD3 EC₅₀ ELISA (nM) | 0.20 | 0.22 | 0.14 | 0.24 | 0.56 | 732.60 | 0.56 | — | 0.20 |
| Fold shiftin EC₅₀ | 1.18 | 1.29 | 0.82 | 1.41 | 3.29 | >1000 | 3.29 | — | 1.18 |
| relative to wt | | | | | | | | | |
| CDR3 HC Ala scan | N | S | Y | I | S | Y | W | A | Y |
| Amino acid position | 106 | 107 | 108 | 109 | 110 | 111 | 112 | — | 114 |
| Mutation tolerated? | T | T | T | MT | MT | NT | NT | — | T |

### Example 9: Tumor Cell Killing with TROP2 TCEs

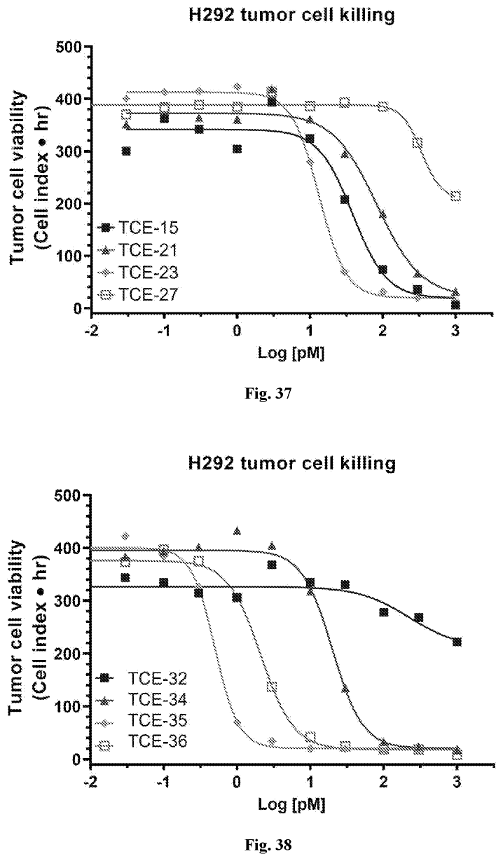

TCEs were evaluated in a functional in vitro tumor cell killing assay using H292, HCT116, or HEK293 cell lines. Recombinant HEK293 cell lines were engineered to express human or cynomolgus monkey TROP2 on their surface. Tumor cell killing was measuring using an ×Celligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight. The following day, TCEs titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of TCE where the concentration required to reduce the tumor growth by 50% (EC₅₀) was calculated using Graphpad Prism software. Data plots of tumor cell viability vs. TCE concentration are shown in FIG. 36 for TCE-9, TCE-11, TCE-12, and TCE-14 mediated killing of H292 tumor cells. FIG. 37 shows data plots of tumor cell viability vs. TCE concentration for TCE-15, TCE-21, TCE-23, and TCE-27 mediated killing of H292 tumor cells. FIG. 38 shows data plots for H292 tumor cell viability vs. TCE concentration for TCE-32, TCE-34, TCE-35, and TCE-36. The EC₅₀s for tumor cell killing of H292 cells by the different TCEs are provided in Tables 36-38.

TABLE 36
TCE Cytotoxicity EC₅₀s (H292 Cells)-Mutated CDRH3

| anti-CD3 scFv | | |
|---|---|---|
| CDRH3 | | H292 Cytotox |
| mutation | TCE Construct | EC₅₀ (pM) |
| wt | TCE-9 | 0.7 |
| R100A | TCE-11 | 3.4 |
| H101A | TCE-12 | 4.4 |
| N103A | TCE-14 | 168.5 |
| F104A | TCE-15 | 40.68 |
| S110A | TCE-21 | 89.76 |
| W112A | TCE-23 | 13.76 |

TABLE 37
TCE Cytotoxicity EC₅₀s (H292 Cells)-Mutated CDRL3

| anti-CD3 scFv | | |
|---|---|---|
| CDRL3 | | H292 Cytotox |
| mutation | TCE Construct | EC₅₀ (pM) |
| wt | TCE-9 | 0.7 |
| W233A | TCE-27 | >1000 |
| W238A | TCE-32 | >1000 |
| F240A | TCE-34 | 19.96 |

TABLE 38
TCE Cytotoxicity EC₅₀s (H292 Cells)

| anti-CD3 scFv | | |
|---|---|---|
| other | | H292 Cytotox |
| mutations | TCE Construct | EC₅₀ (pM) |
| wt | TCE-9 | 0.7 |
| P41S, A49G, | TCE-35 | 0.5 |
| N87S, L150F, | | |
| T151S, G163R, | | |
| P175A, K181T, | | |
| T200V, A202D, | | |
| L208I, G211N, | | |
| L217I, S218T, | | |
| V220A, P222A, | | |
| E223D, A226S, | | |
| E227D | | |
| relative to wt: | TCE-36 | 2.1 |
| N30Q, I109V, | | |
| G172A, V231A | | |

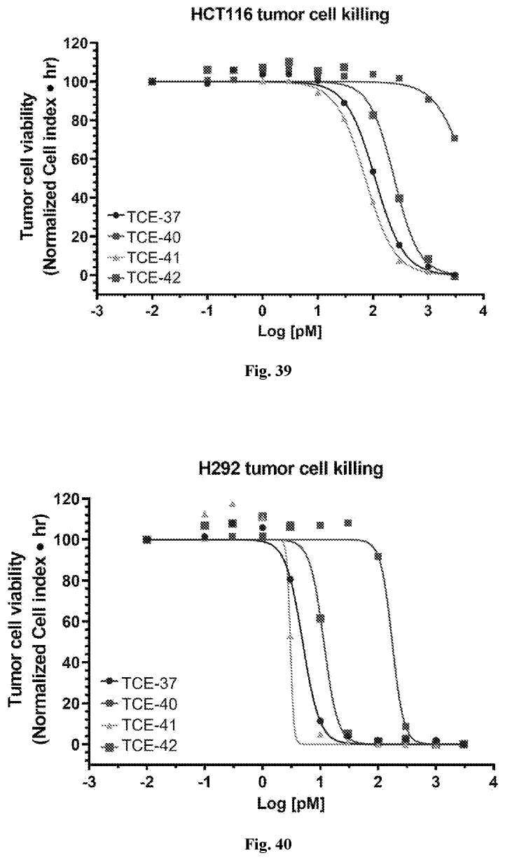

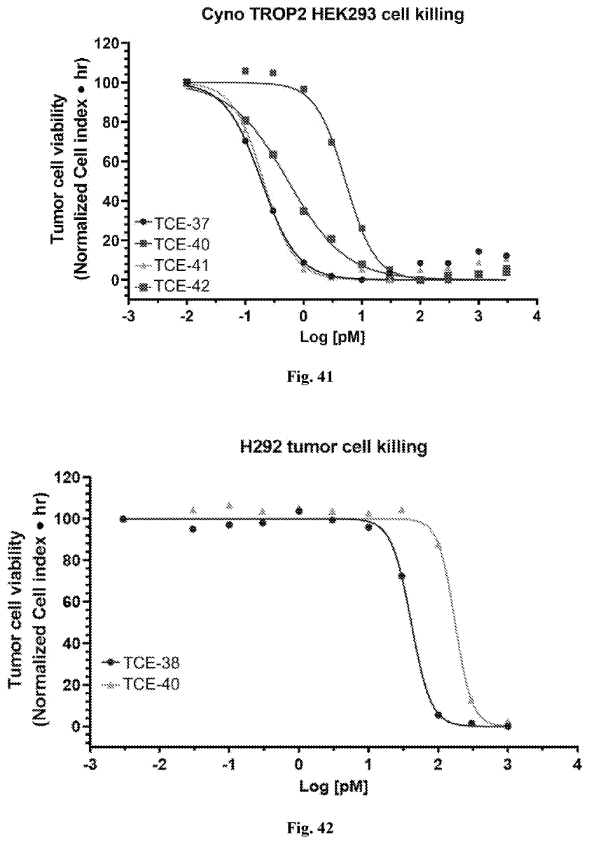

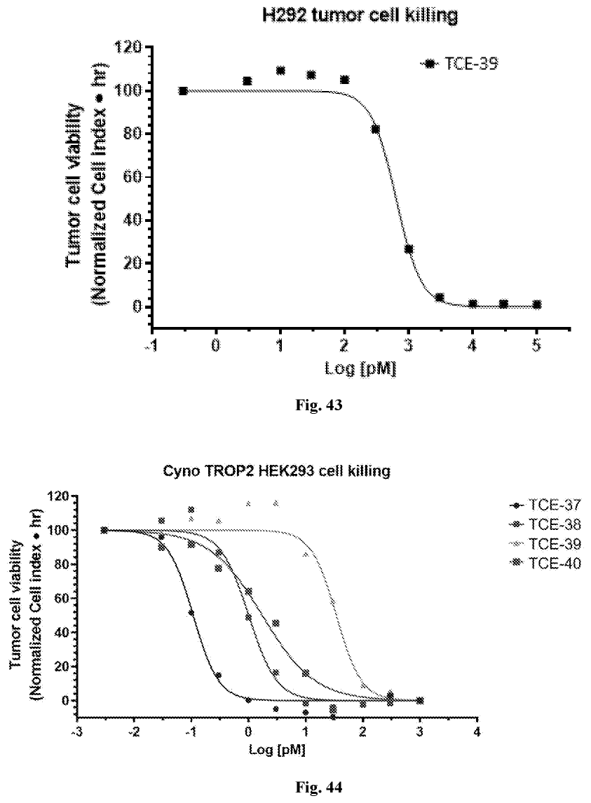

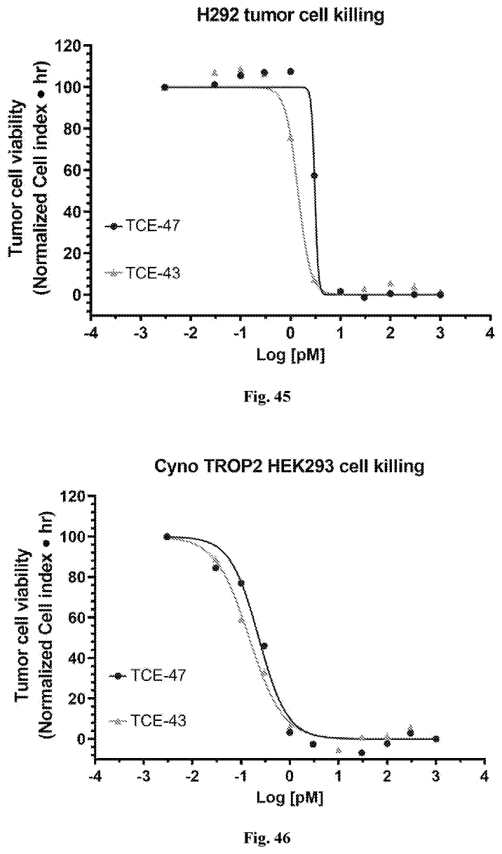

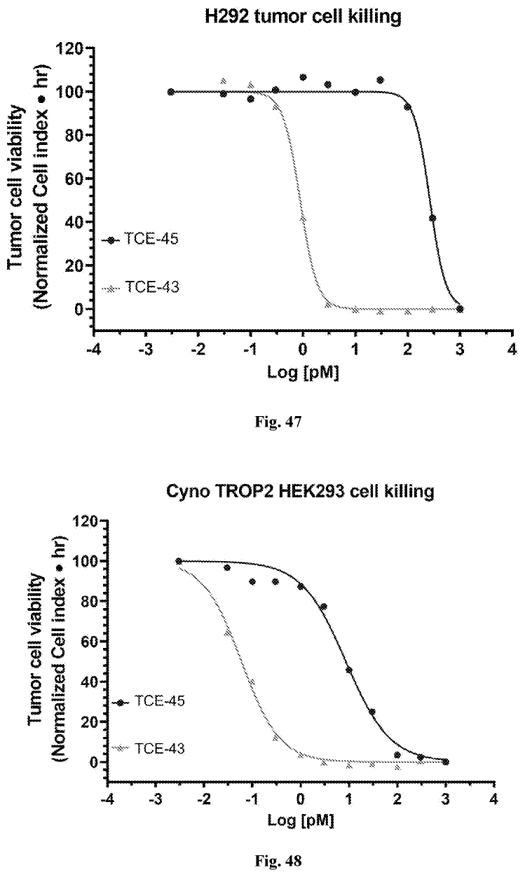

Data plots for tumor cell killing with TCE-37, TCE-40, TCE-41, and TCE-42 are shown in FIG. 39 (HCT116 cells), FIG. 40 (H292 cells), and FIG. 41 (cyno TROP2 HEK293 cells). Data plots for H292 tumor cell killing with TCE-38 and TCE-40 are shown in FIG. 42. A data plot for H292 tumor cell killing with TCE-39 is shown in FIG. 43. Data plots for cyno TROP2 HEK293 cell killing with TCE-37, TCE-38, TCE-39, and TCE-40 are shown in FIG. 44. Data plots for tumor cell killing with TCE-47 and TCE-43 are shown in FIG. 45 (H292 cells) and FIG. 46 (cyno TROP2 HEK293 cells). Data plots for tumor cell killing with TCE-45 and TCE-43 are shown in FIG. 47 (H292 cells) and FIG. 48 (cyno TROP2 HEK293 cells). The EC₅₀s for cytotoxicity of the different TCEs are provided in Tables 39-41.

TABLE 39
TCE Cytotoxicity EC₅₀s (HCT116 Cells)

| Anti-CD3 scFv | TCE | HCT116 Cytotox |
|---|---|---|
| mutations | Construct | EC₅₀ (pM) |
| wt | TCE-37 | 109 |
| F240A | TCE-40 | >3,000 |
| P41S, A49G, | TCE-41 | 73 |
| N87S, L150F, | | |
| T151S, G163R, | | |
| P175A, K181T, | | |
| T200V, A202D, | | |
| L208I, G211N, | | |
| L217I, S218T, | | |
| V220A, P222A, | | |
| E223D, A226S, | | |
| E227D | | |
| N30Q, I109V, | TCE-42 | 243 |
| G172A, V231A | | |

TABLE 40
TCE Cytotoxicity EC₅₀s (H292 Cells)

| Anti-CD3 scFv | TCE | H292 Cytotox |
|---|---|---|
| mutations | Construct | EC₅₀ (pM) |
| wt | TCE-37 | 5 |
| F240A | TCE-40 | 175 |
| P41S, A49G, | TCE-41 | 3 |
| N87S, L150F, | | |
| T151S, G163R, | | |
| P175A, K181T, | | |
| T200V, A202D, | | |
| L208I, G211N, | | |
| L217I, S218T, | | |
| V220A, P222A, | | |
| E223D, A226S, | | |
| E227D | | |
| N30Q, 1109V, | TCE-42 | 11 |
| G172A, V231A | | |
| H101A | TCE-38 | 41 |
| F104A | TCE-39 | 632 |
| P41S, A49G, | TCE-47 | 3 |
| N87S, L150F, | | |
| T151S, G163R, | | |
| P175A, K181T, | | |
| T200V, A202D, | | |
| L208I, G211N, | | |
| L217I, S218T, | | |
| V220A, P222A, | | |
| E223D, A226S, | | |
| E227D | | |
| wt | TCE-43 | 1 |
| F104A | TCE-45 | 266 |

TABLE 41
TCE Cytotoxicity EC₅₀s (cynoTROP2 + HEK293 cells)

| Anti-CD3 scFv | TCE | CynoTROP2 + HEK293 cells |
|---|---|---|
| mutations | Construct | Cytotox EC₅₀ (pM) |
| wt | TCE-37 | 0.2 |
| F240A | TCE-40 | 5 |
| P41S, A49G, N87S, | TCE-41 | 0.2 |
| L150F, T151S, | | |
| G163R, P175A, | | |
| K181T, T200V, | | |
| A202D, L208I, | | |
| G211N, L217I, | | |
| S218T, V220A, | | |
| P222A, E223D, | | |
| A226S, E227D | | |
| N30Q, I109V, | TCE-42 | 0.5 |
| G172A, V231A | | |
| H101A | TCE-38 | 1 |
| F104A | TCE-39 | 34 |
| N30Q, I109V, | TCE-47 | 0.2 |
| G172A, V231A | | |
| wt | TCE-43 | 0.1 |
| F104A | TCE-45 | 9 |

### Example 10: Non-Human Primate (NHP) Studies of TROP2 TCEs

### NHP Toxicity Studies of TCE-37 and TCE-43

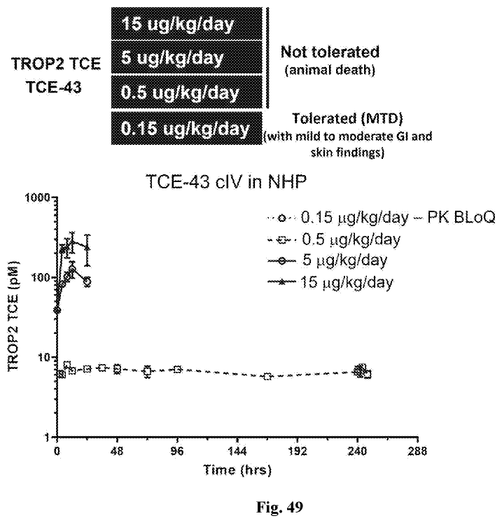

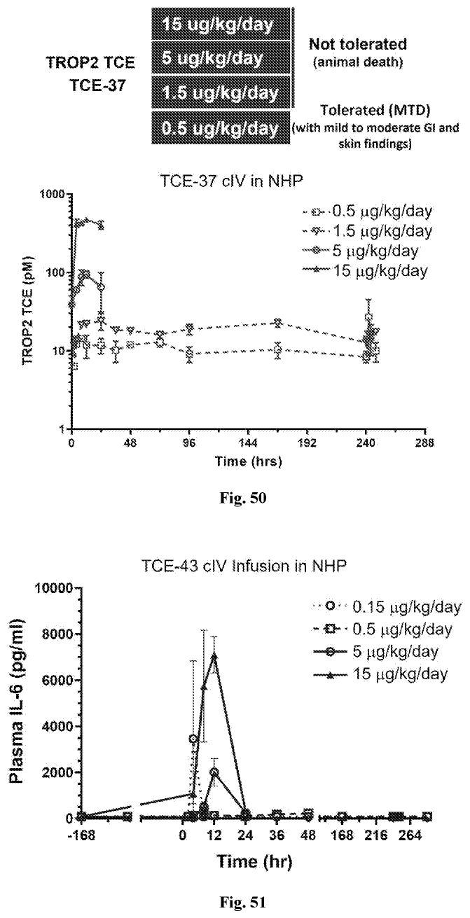

Pharmacokinetics and exploratory safety of TCE-37 (wt anti-CD3, anti-TROP2 F108A mutant) and TCE-43 (wt anti-CD3, anti-TROP2 D109A mutant) were evaluated in cynomolgus monkeys. Briefly, monkeys of approximately 3 kilograms (kg) bodyweight were administered TCEs as a continuous IV (cIV) infusion. Animals were observed for signs of adverse events. TCE-43 was dosed at 0.15 micrograms/kilogram/day (μg/kg/day), 0.5 μg/kg/day, 5 μg/kg/day, and 15 μg/kg/day. TCE-37 was dosed at 0.5 μg/kg/day, 1.5 μg/kg/day, 5 μg/kg/day, and 15 μg/kg/day. After dosing, blood was collected in K2 EDTA tubes at specific time points and processed to plasma. Plasma was frozen until analysis. Concentrations of TCEs in plasma were measured via a Meso Scale Discovery (MSD) based method relative to a reference standard diluted in control cynomolgus monkey plasma. Pharmacokinetic profiles and toxicity results are shown in FIG. 49 (TCE-43) and FIG. 50 (TCE-37). Both TCE-43 and TCE-37 showed a dose proportional exposure increase in general. As shown in the figures, the maximum tolerated doses (MTD) for TCE-43 and TCE-37 were 0.15 μg/kg/day and 0.5 μg/kg/day, respectively. Mild to moderate gastrointestinal (GI) and skin findings were observed at these doses.

Cytokine Induction in NHPs with TCE-37 and TCE-43

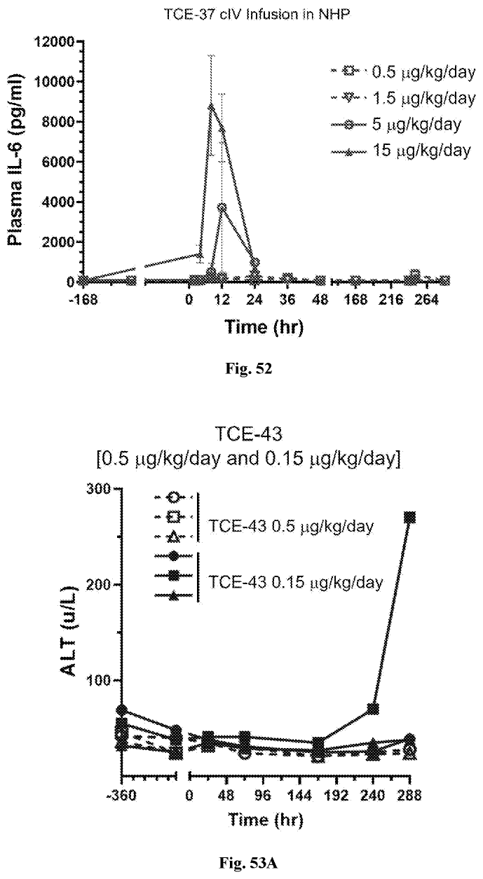

Cytokine release after TCE-37 and TCE-43 administration by cIV was evaluated in cynomolgus monkeys. Briefly, cynomolgus monkeys of approximately 3 kg bodyweight were implanted with an infusion pump subcutaneously. Two weeks later the pump was filled with TCE dosing solution and administered via constant infusion. After dosing started, blood was collected in K2 EDTA tubes at specific timepoints and processed to plasma. Plasma samples were analyzed for cytokines using a non-human primate cytometric Th1/Th2 bead array kit from BD biosciences following the manufacturer's instructions. Interleukin 6 (IL-6) levels in plasma were calculated relative to reference standards provided with the bead array kit. FIGS. 51-52 show IL-6 release in cynomolgus monkey after continuous infusion of TCE-43 (FIG. 51) and TCE-37 (FIG. 52) at different doses. IL-6 levels (in picograms/milliliter (pg/mL)) following cIV infusion for 10 days at different doses are provided in Table 42 for TCE-43 and Table 43 for TCE-37.

TABLE 42
NHP Continuous IV Infusion of TCE-43 for 10 Days

| 0.15 μg/kg/day | 0.5 μg/kg/day | 5 μg/kg/day | 15 μg/kg/day |
|---|---|---|---|
| 3458 pg/mL IL-6 | 298 pg/mL IL-6 | 2006 pg/mL IL-6 | 7098 pg/mL IL-6 |

TABLE 43
NHP Continuous IV Infusion of TCE-37 for 10 Days

| 0.5 μg/kg/day | 1.5 μg/kg/day | 5 μg/kg/day | 15 μg/kg/day |
|---|---|---|---|
| 356 pg/mL IL-6 | 324 pg/mL IL-6 | 3719 pg/mL IL-6 | 8811 pg/mL IL-6 |

### Clinical Chemistry of TCE-37 and TCE-43 in NHP

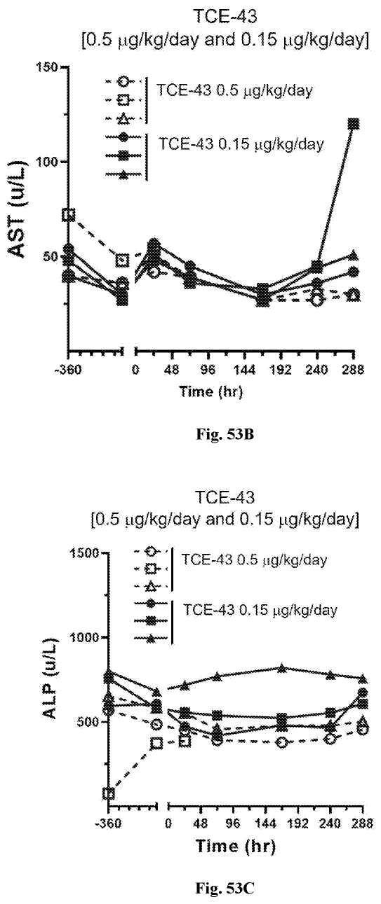

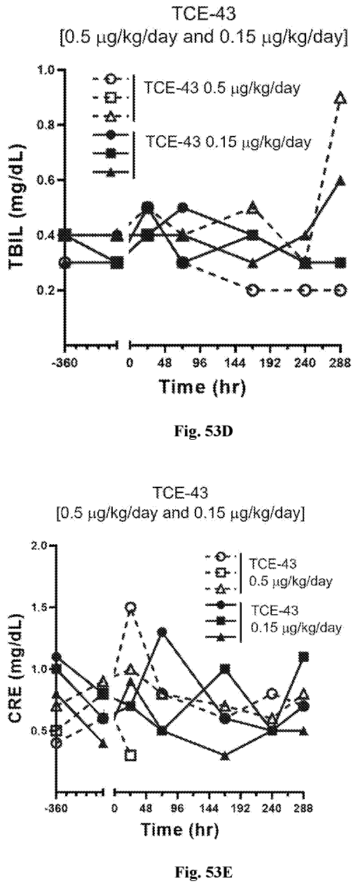

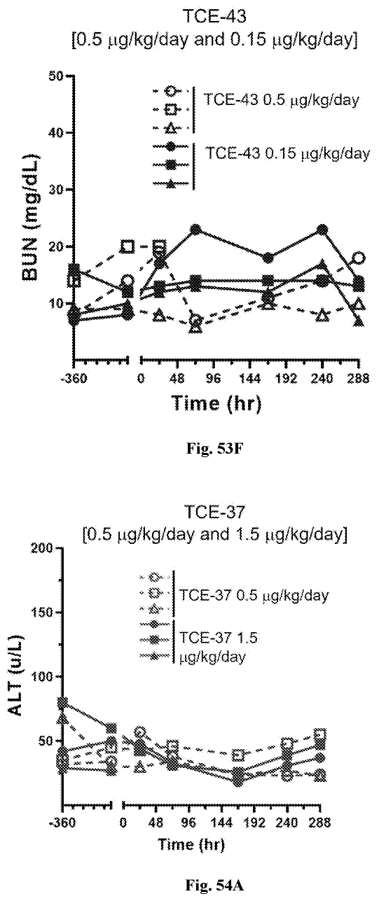

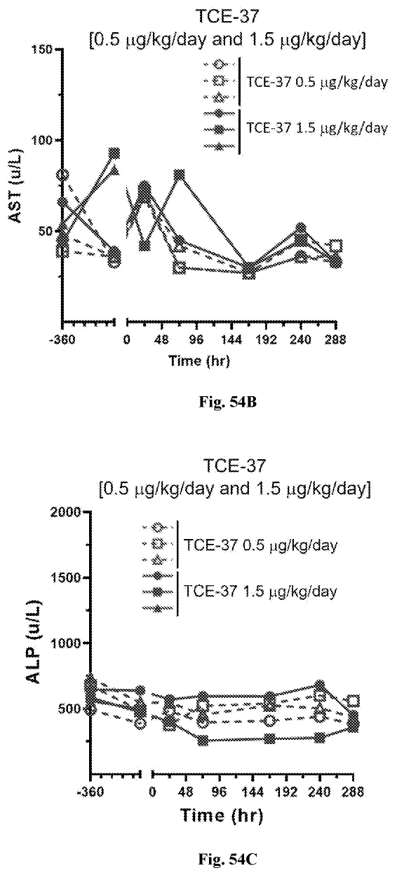

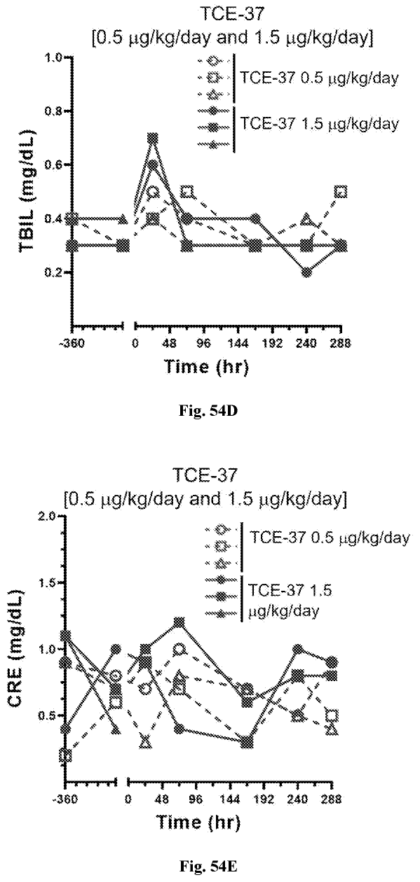

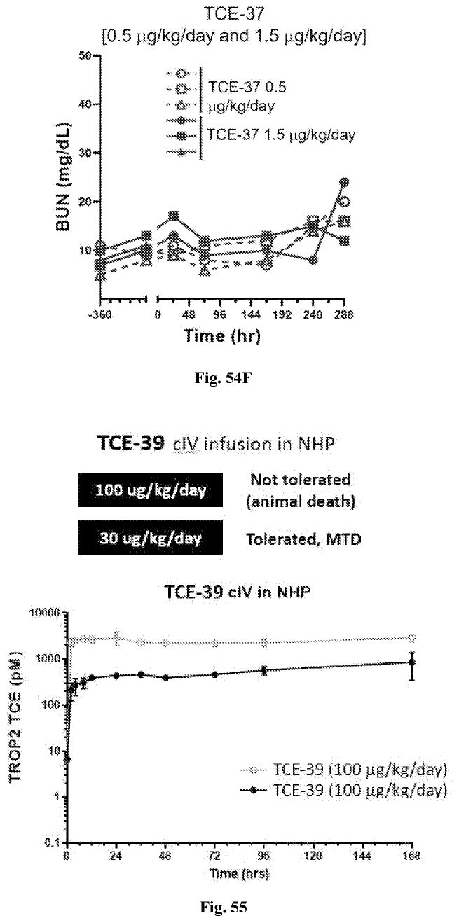

Clinical chemistry after TCE-37 and TCE-43 administration by cIV in cynomolgus monkeys was measured through standard panel analyses. Briefly, cynomolgus monkeys of approximately 3 kg bodyweight were administered TCEs by cIV and observed daily for signs of adverse events. After dosing, blood was collected in K2 EDTA tubes at specific timepoints and processed to plasma. Clinical chemistry parameters were run on freshly prepared plasma samples, including alanine aminotransferase (ALT), aspartate transaminase (AST), alkaline phosphatase (ALP), bilirubin (TBIL), creatinine (CRE), and blood urea nitrogen (BUN) as signs of liver and kidney related toxicity. FIGS. 53-54 show plots of ALT, AST, ALP, TBIL, CRE, and BUN levels in cynomolgus monkeys after administration of TCE-43 (FIG. 53A-53F) and TCE-37 (FIG. 54A-54F).

### NHP Toxicity Studies of TCE-39, TCE-40, and TCE-42

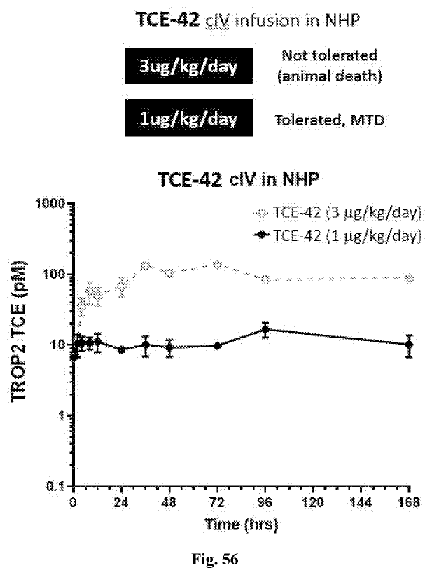

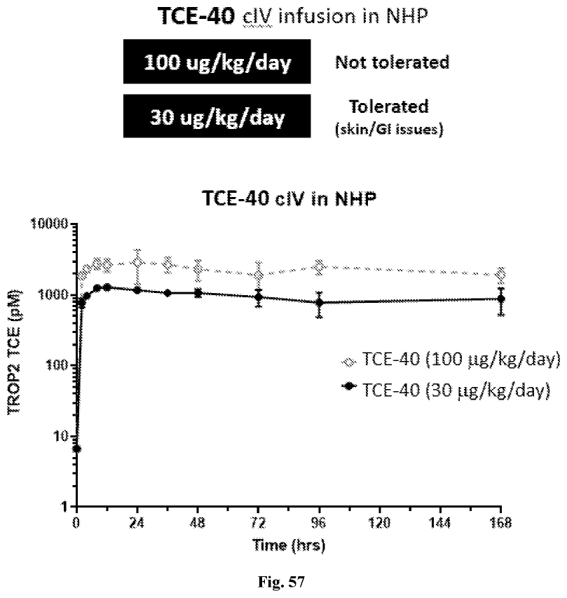

Pharmacokinetics and exploratory safety of TCE-39, TCE-40, and TCE-42 were evaluated as described above. Pharmacokinetic profiles and toxicity results are shown in FIG. 55 (TCE-39), FIG. 56 (TCE-42), and FIG. 57 (TCE-40). As shown in the figures, maximum tolerated doses were 30 g/kg/day for TCE-39, 1 μg/kg/day for TCE-42, and 30 g/kg/day for TCE-40.

Cytokine Induction in NHPs with TCE-39, TCE-40, and TCE-42

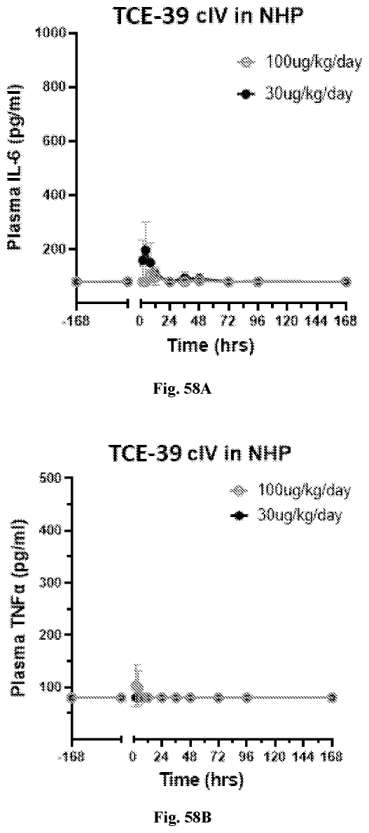

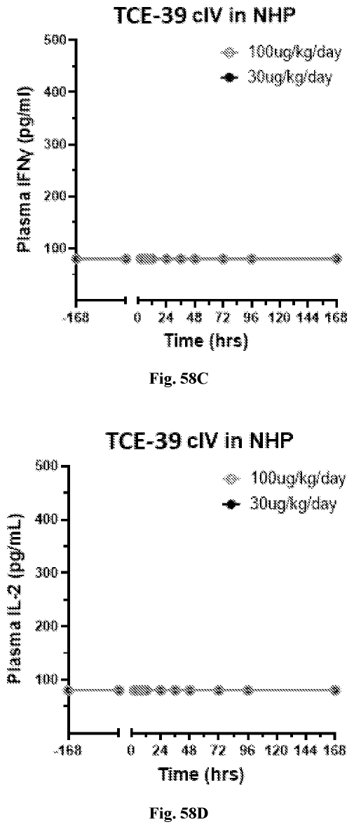

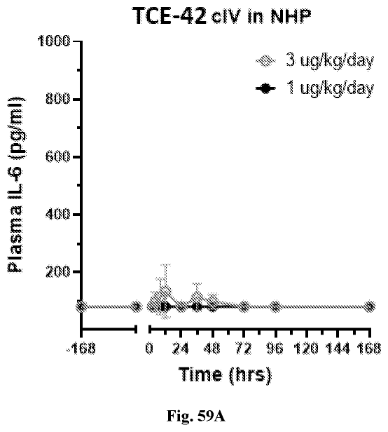

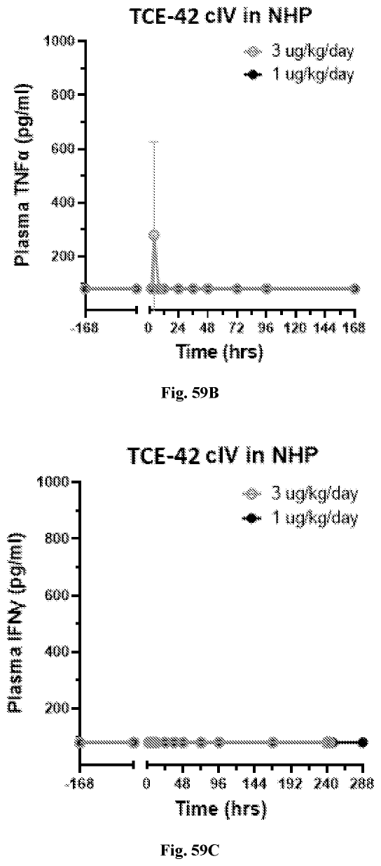

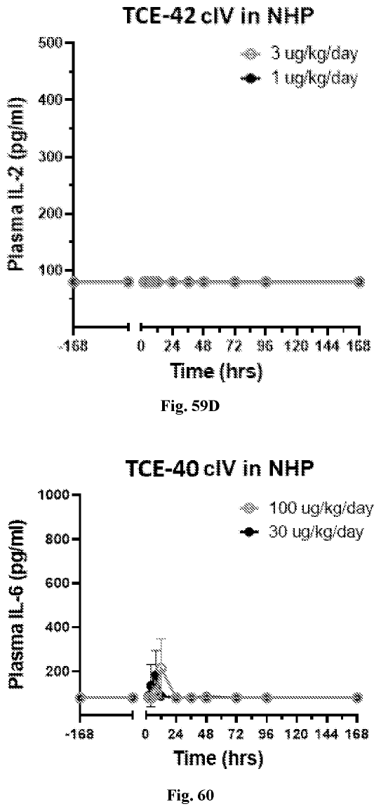

Cytokine release after TCE-39, TCE-40, and TCE-42 administration by cIV were evaluated in cynomolgus monkeys as described above. FIGS. 58A-58D show release of IL-6, tumor necrosis factor alpha (TNFα), interferon gamma (IFNγ), and interleukin-2 (IL-2) in cynomolgus monkey after continuous infusion of TCE-39. FIGS. 59A-59D show release of IL-6, TNFα, IFNγ, and IL-2 in cynomolgus monkey after continuous infusion of TCE-42. FIG. 60 shows release of IL-6 in cynomolgus monkey after continuous infusion of TCE-40. IL-6 levels in pg/mL following cIV infusion for 10 days at different doses are provided in Table 44 for TCE-39, Table 45 for TCE-40, and Table 46 for TCE-42.

TABLE 44
NHP Continuous IV Infusion of TCE-39 for 10 Days

| 30 μg/kg/day | 100 μg/kg/day |
|---|---|
| 197 pg/mL IL-6 | 100 pg/mL IL-6 |

TABLE 45
NHP Continuous IV Infusion of TCE-40 for 10 Days

| 30 μg/kg/day | 100 μg/kg/day |
|---|---|
| 183 pg/mL IL-6 | 213 pg/mL IL-6 |

TABLE 46
NHP Continuous IV Infusion of TCE-42 for 10 Days

| 1 μg/kg/day | 3 μg/kg/day |
|---|---|
| IL-6, below limit | 133 pg/mL IL-6 |
| of quantitation | |

### Clinical Chemistry of TCE-39, TCE-40, and TCE-42

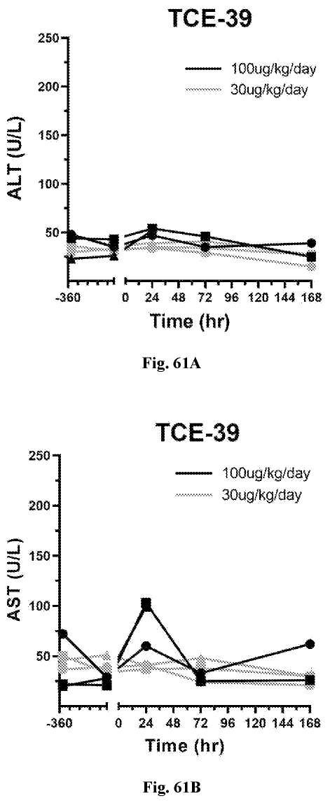

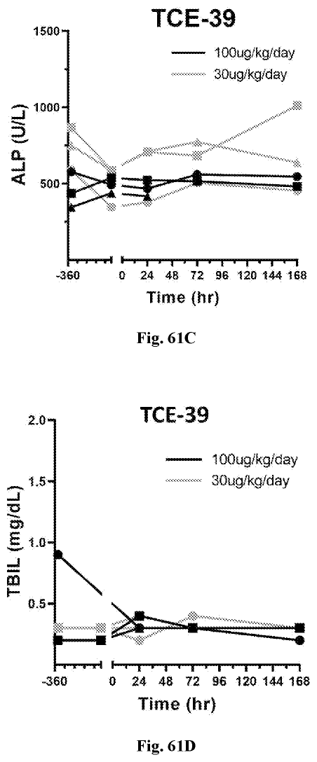

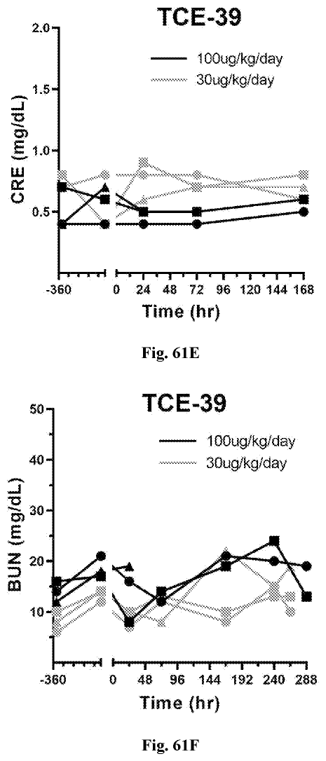

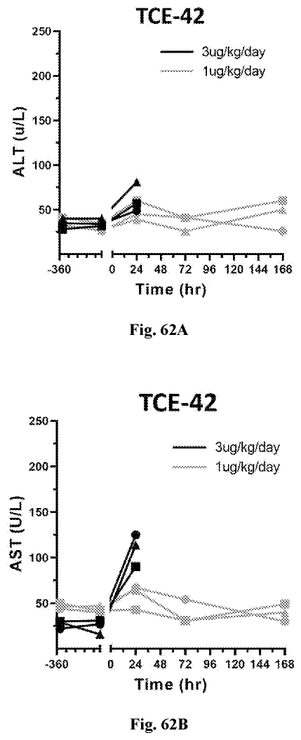

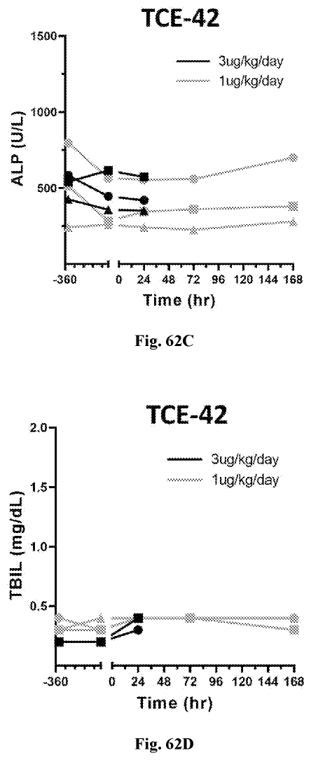

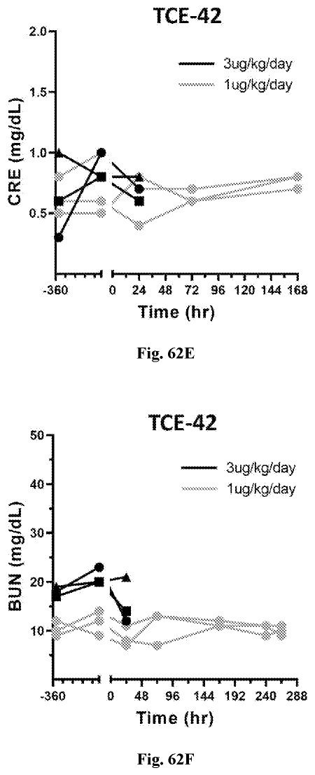

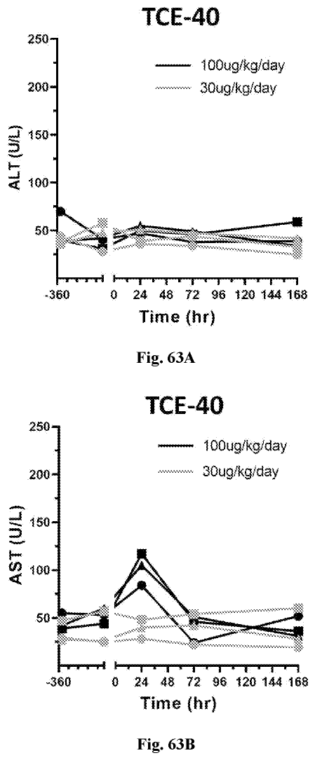

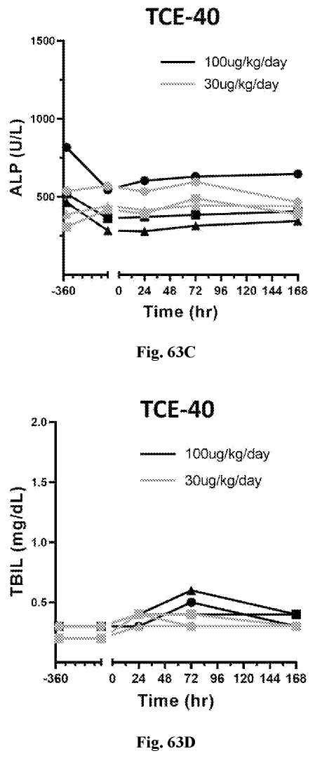

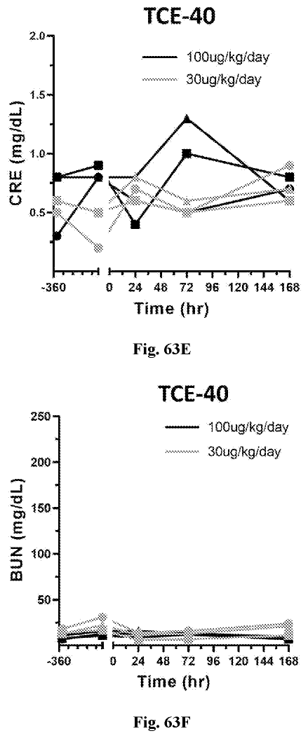

Clinical chemistry after TCE-39, TCE-40, and TCE-42 administration by cIV in cynomolgus monkeys was measured through standard panel analyses as described above. FIGS. 61-63 show plots of ALT, AST, ALP, TBIL, CRE, and BUN levels in cynomolgus monkeys after administration of TCE-39 (FIGS. 61A-61F), TCE-42 (FIGS. 62A-62F), and TCE-40 (FIGS. 63A-63F).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H,
wherein CDR1-L comprises the amino acid sequence of SEQ ID NO: 1;
wherein CDR2-L comprises the amino acid sequence of SEQ ID NO: 2 (SA); and
wherein CDR3-L comprises an amino acid sequence of X₁X₂HYX₃X₄X₅X₆X₇;
wherein X₁ is Q, S, T, D, N, E, H, K, R, or A;
X₂ is Q, S, T, D, N, E, H, K, R, or A;
X₃ is I, G, P, V, L, M, S, T, or A;
X₄ is T, G, S, M, H, N, Q, or A;
X₅ is P, G, V, L, I, M, S, T, or A;
X₆ is L, G, P, V, I, M, S, T, or A; and
X₇ is T, G, S, M, H, N, Q, or A;
wherein CDR1-H comprises the amino acid sequence of SEQ ID NO: 13;
wherein CDR2-H comprises the amino acid sequence of SEQ ID NO: 14; and
wherein CDR3-H comprises an amino acid sequence of AX₈X₉GX₁₀X₁₁X₁₂X₁₃YWX₁₄X₁₅X₁₆X₁₇;
wherein X₈ is R, S, T, Q, D, E, H, K, N, H, K, or A;
X₉ is G, P, V, L, I, M, S, T, or A;
X₁₀ is F, Y, W, V, L, I, G, or A;
X₁₁ is G, P, V, L, I, M, S, T, or A;
X₁₂ is S, G, T, M, N, Q, H, or A;
X₁₃ is S, G, T, M, N, Q, H, or A;
X₁₄ is Y, F, W, V, L, I, G, or A;
X₁₅ is F, Y, W, V, L, I, G, or A;
X₁₆ is D, Q, N, E, S, T, H, K, R, or A; and
X₁₇ is V, G, P, L, I, M, S, T, or A;
wherein when CDR3-H comprises the amino acid sequence of SEQ ID NO: 15, then CDR3-L does not comprise the amino acid sequence of SEQ ID NO: 3.

2. The recombinant antibody or antigen binding fragment thereof of claim 1, wherein
X₁ is Q, N, D, E, or A;
X₂ is Q, N, D, E, or A;
X₃ is I, V, L, or A;
X₄ is T, S, or A;
X₅ is P, G, or A;
X₆ is L, V, I, or A;
X₇ is T, S, or A;
X₈ is R, K, or A;
X₉ is G, S, T, or A;
X₁₀ is F, Y, or A;
X₁₁ is G, S, T, or A;
X₁₂ is S, G, T, or A;
X₁₃ is S, G, T, or A;
X₁₄ is Y, W, F, or A;
X₁₅ is F, Y, W, or A;
X₁₆ is D, E, Q, N, or A; and
X₁₇ is V, L, I, or A.

3. The recombinant antibody or antigen binding fragment thereof of claim 1, wherein
X₁ is Q; and
X₆ is L.

4. The recombinant antibody or antigen binding fragment thereof of claim 3, wherein
X₈ is R;
X₁₀ is F;
X₁₁ is G;
X₁₄ is Y;
X₁₅ is F; and
X₁₆ is D.

5. The recombinant antibody or antigen binding fragment thereof of claim 1, wherein
X₁ is Q, S, T, D, N, E, or A;
X₂ is Q, S, T, D, N, E, or A;
X₃ is I, G, P, V, L, M, or A;
X₄ is T, G, S, M, H, N, Q, or A;
X₅ is P, G, V, L, I, M, or A;
X₆ is L, G, P, V, I, M, or A;
X₇ is T, G, S, M, H, N, Q, or A;
X₈ is R, H, K, or A;
X₉ is G, P, V, L, I, M, S, T, or A;
X₁₀ is F, Y, W, V, L, I, or A;
X₁₁ is G, P, V, L, I, M, S, T, or A;
X₁₂ is S, G, T, M, N, Q, or A;
X₁₃ is S, G, T, M, N, Q, or A;
X₁₄ is Y, F, W, V, L, I, or A;
X₁₅ is F, Y, W, V, L, I, or A;
X₁₆ is D, Q, N, E, S, T, or A; and
X₁₇ is V, G, P, L, I, M, or A.

6. The recombinant antibody or antigen binding fragment thereof of claim 5, wherein
X₁ is Q, N, or A;
X₂ is Q, N, or A;
X₃ is I, V, L, or A;
X₄ is T, S, or A;
X₅ is P, G, or A;
X₆ is L, V, I, or A;
X₇ is T, S, or A;
X₈ is R, K, or A;
X₉ is G, V, S, T, or A;
X₁₀ is F, Y, or A;
X₁₁ is G, V, S, T, or A;
X₁₂ is S, G, T, or A;
X₁₃ is S, G, T, or A;
X₁₄ is Y, W, or A;
X₁₅ is F, Y, or A;
X₁₆ is D, E, or A; and
X₁₇ is V, G, L, I, or A.

7. The recombinant antibody or antigen binding fragment thereof of claim 5, wherein
X₁ is Q; and
X₆ is L.

8. The recombinant antibody or antigen binding fragment thereof of claim 7, wherein
X₈ is R;
X₁₀ is F;
X₁₁ is G;
X₁₄ is Y;
X₁₅ is F; and
X₁₆ is D.

9. The recombinant antibody or antigen binding fragment thereof of claim 1, wherein CDR3-L comprises an amino acid selected from SEQ ID NOs: 3-5 and 8-12.

10. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (SA), CDR3-L: SEQ ID NO: 4, and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 5 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 8 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 9 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 10 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 11 and CDR1-H: SEQ ID NO: 13, CDR2-H: SEQ ID NO: 14, CDR3-H: SEQ ID NO: 15; and
CDR1-L: SEQ ID NO: 1, CDR2-L: SEQ ID NO: 2 (SA), CDR3-L: SEQ ID NO: 12 and CDR1-H: SEQ ID NO: 13, CDR2-H: SEQ ID NO: 14, CDR3-H: SEQ ID NO: 15.

11. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (SA), CDR3-L: SEQ ID NO: 5 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 8 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 9 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 10 and CDR1-H: SEQ ID NO: 13, CDR2-H: SEQ ID NO: 14, CDR3-H: SEQ ID NO: 15; and
CDR1-L: SEQ ID NO: 1, CDR2-L: SEQ ID NO: 2 (SA), CDR3-L: SEQ ID NO: 12 and CDR1-H: SEQ ID NO: 13, CDR2-H: SEQ ID NO: 14, CDR3-H: SEQ ID NO: 15.

12. The recombinant antibody or antigen binding fragment thereof of claim 1, wherein CDR3-H comprises an amino acid selected from SEQ ID NOs: 16-17, 19-22, and 25-28.

13. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14; CDR3-H: SEQ ID NO: 16;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 17;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 19;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 20;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 21;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 22;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 25;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 26;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 27; and
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 28.

14. The recombinant antibody or antigen binding fragment thereof of claim 1, wherein the immunoglobulin light chain and the immunoglobulin heavy chain comprise a set of CDR sequences selected from the group consisting of CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 17;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 21;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 22; and
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 28.

15. A recombinant antibody or antigen binding fragment thereof that comprises a tumor-associated calcium signal transducer 2 (TROP2) binding domain, wherein the TROP2 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (SA), CDR3-L: SEQ ID NO: 4, and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 5 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 6 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 7 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 8 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 9 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 10 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 11 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 12 and CDR1-H: SEQ ID NO: 13, CDR2-H: SEQ ID NO: 14, CDR3-H: SEQ ID NO: 15;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 16;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 17;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 18;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 19;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 20;
CDR1-L: SEQ ID NO: 1, CDR2-L: SEQ ID NO: 2 (SA); CDR3-L: SEQ ID NO: 3, and CDR1-H: SEQ ID NO: 13, CDR2-H: SEQ ID NO: 14, CDR3-H: SEQ ID NO: 21;
CDR1-L: SEQ ID NO: 1, CDR2-L: SEQ ID NO: 2 (SA); CDR3-L: SEQ ID NO: 3, and CDR1-H: SEQ ID NO: 13, CDR2-H: SEQ ID NO: 14; CDR3-H: SEQ ID NO: 22;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 23;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 24;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 25;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 26;
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 27; and
CDR1-L: SEQ ID NO: 1, CDR2-L: SEQ ID NO: 2 (SA), CDR3-L: SEQ ID NO: 3, and CDR1-H: SEQ ID NO: 13, CDR2-H: SEQ ID NO: 14, CDR3-H: SEQ ID NO: 28.

16. The recombinant antibody or antigen binding fragment thereof of claim 1, wherein the TROP2 binding domain comprises a Fab, Fab′, (Fab′)₂ or a single chain variable fragment (scFv).

17. The recombinant antibody or antigen binding fragment thereof of claim 1, wherein the TROP2 binding domain is a Fab.

18. The recombinant antibody or antigen binding fragment thereof of claim 1, wherein the immunoglobulin light chain comprises a variable domain of an immunoglobulin kappa (IgK) or immunoglobulin lambda (IgL) light chain.

19. The recombinant antibody or antigen binding fragment thereof of claim 1, wherein the immunoglobulin heavy chain comprises a variable domain of an IgG1, IgG2, IgG3, or IgG4 heavy chain.

20. The recombinant antibody or antigen binding fragment thereof of claim 1, wherein the immunoglobulin light chain comprises an amino acid sequence with at least 90%, 95%, 96%, 97%, 98%, 99%, or 100% identity to any one of SEQ ID NOs: 31, 33, 35, 37, 39, 41, 43, 45, 47, 49, 51, 53, 55, 57, 59, 61, 63, 65, 67, 69, 71, or 73.

21. The recombinant antibody or antigen binding fragment thereof of claim 1, wherein the immunoglobulin heavy chain comprises an amino acid sequence with at least 90%, 95%, 96%, 97%, 98%, 99%, or 100% identity to any one of SEQ ID NOs: 32, 34, 36, 38, 40, 42, 44, 46, 48, 50, 52, 54, 56, 58, 60, 62, 64, 66, 68, 70, 72, or 74.

22. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 31 and the immunoglobulin heavy chain comprises an amino acid sequence with at least 90%, 95%, 96%, 97%, 98%, 99%, or 100% identity to SEQ ID NO: 32.

23. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 33 and the immunoglobulin heavy chain comprises an amino acid sequence with at least 90%, 95%, 96%, 97%, 98%, 99%, or 100% identity to SEQ ID NO: 34.

24. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 35 and the immunoglobulin heavy chain comprises an amino acid sequence with at least 90%, 95%, 96%, 97%, 98%, 99%, or 100% identity to SEQ ID NO: 36.

25. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 37 and the immunoglobulin heavy chain comprises an amino acid sequence with at least 90%, 95%, 96%, 97%, 98%, 99%, or 100% identity to SEQ ID NO: 38.

26. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 39 and the immunoglobulin heavy chain comprises an amino acid sequence with at least 90%, 95%, 96%, 97%, 98%, 99%, or 100% identity to SEQ ID NO: 40.

27. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 41 and the immunoglobulin heavy chain comprises an amino acid sequence with at least 90%, 95%, 96%, 97%, 98%, 99%, or 100% identity to SEQ ID NO: 42.

28. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 43 and the immunoglobulin heavy chain comprises an amino acid sequence with at least 90%, 95%, 96%, 97%, 98%, 99%, or 100% identity to SEQ ID NO: 44.

29. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 45 and the immunoglobulin heavy chain comprises an amino acid sequence with at least 90%, 95%, 96%, 97%, 98%, 99%, or 100% identity to SEQ ID NO: 46.

30. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 47 and the immunoglobulin heavy chain comprises an amino acid sequence with at least 90%, 95%, 96%, 97%, 98%, 99%, or 100% identity to SEQ ID NO: 48.

31. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 49 and the immunoglobulin heavy chain comprises an amino acid sequence with at least 90%, 95%, 96%, 97%, 98%, 99%, or 100% identity to SEQ ID NO: 50.

32. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 51 and the immunoglobulin heavy chain comprises an amino acid sequence with at least 90%, 95%, 96%, 97%, 98%, 99%, or 100% identity to SEQ ID NO: 52.

33. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 53 and the immunoglobulin heavy chain comprises an amino acid sequence with at least 90%, 95%, 96%, 97%, 98%, 99%, or 100% identity to SEQ ID NO: 54.

34. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 55 and the immunoglobulin heavy chain comprises an amino acid sequence with at least 90%, 95%, 96%, 97%, 98%, 99%, or 100% identity to SEQ ID NO: 56.

35. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 57 and the immunoglobulin heavy chain comprises an amino acid sequence with at least 90%, 95%, 96%, 97%, 98%, 99%, or 100% identity to SEQ ID NO: 58.

36. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 59 and the immunoglobulin heavy chain comprises an amino acid sequence with at least 90%, 95%, 96%, 97%, 98%, 99%, or 100% identity to SEQ ID NO: 60.

37. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 61 and the immunoglobulin heavy chain comprises an amino acid sequence with at least 90%, 95%, 96%, 97%, 98%, 99%, or 100% identity to SEQ ID NO: 62.

38. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 63 and the immunoglobulin heavy chain comprises an amino acid sequence with at least 90%, 95%, 96%, 97%, 98%, 99%, or 100% identity to SEQ ID NO: 64.

39. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 65 and the immunoglobulin heavy chain comprises an amino acid sequence with at least 90%, 95%, 96%, 97%, 98%, 99%, or 100% identity to SEQ ID NO: 66.

40. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 67 and the immunoglobulin heavy chain comprises an amino acid sequence with at least 90%, 95%, 96%, 97%, 98%, 99%, or 100% identity to SEQ ID NO: 68.

41. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 69 and the immunoglobulin heavy chain comprises an amino acid sequence with at least 90%, 95%, 96%, 97%, 98%, 99%, or 100% identity to SEQ ID NO: 70.

42. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 71 and the immunoglobulin heavy chain comprises an amino acid sequence with at least 90%, 95%, 96%, 97%, 98%, 99%, or 100% identity to SEQ ID NO: 72.

43. The recombinant antibody or antigen binding fragment thereof of claim 1, wherein the immunoglobulin light chain comprises an amino acid sequence with at least 90%, 95%, 96%, 97%, 98%, 99%, or 100% identity to SEQ ID NO: 73 and the immunoglobulin heavy chain comprises an amino acid sequence with at least 90%, 95%, 96%, 97%, 98%, 99%, or 100% identity to SEQ ID NO: 74.

44. The recombinant antibody or antigen binding fragment thereof of claim 1, wherein the TROP2 binding domain has weaker binding to TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions.

45. The recombinant antibody or antigen binding fragment thereof of claim 1, wherein the TROP2 binding domain has an increased EC₅₀ for TROP2 as compared to a TROP2 binding domain that comprises an immunoglobulin light chain according to SEQ ID NO: 29 and an immunoglobulin heavy chain according to SEQ ID NO: 30 as measured by ELISA in substantially similar assay conditions.

46. The recombinant antibody or antigen binding fragment thereof of claim 1, wherein the TROP2 binding domain has a faster off rate (larger k_{diss}) for TROP2 binding as compared to a TROP2 binding domain that comprises an immunoglobulin light chain according to SEQ ID NO: 29 and an immunoglobulin heavy chain according to SEQ ID NO: 30 as measured under substantially similar kinetic assay conditions.

47. The recombinant antibody or antigen binding fragment thereof of claim 1, wherein the recombinant antibody or antigen binding fragment further comprises a CD3 binding domain.

48. The recombinant antibody or antigen binding fragment thereof of claim 47, wherein the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H,
wherein CDR1-L comprises the amino acid sequence of SEQ ID NO: 75;
wherein CDR2-L comprises the amino acid sequence of SEQ ID NO: 76;
wherein CDR3-L comprises the amino acid sequence of SEQ ID NO: 77;
wherein CDR1-H comprises the amino acid sequence of SEQ ID NO: 78;
wherein CDR2-H comprises the amino acid sequence of SEQ ID NO: 79; and
wherein CDR3-H comprises the amino acid sequence of SEQ ID NO: 80.

49. The recombinant antibody or antigen binding fragment thereof of claim 47, wherein the CD3 binding domain comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H,
wherein CDR1-L comprises the amino acid sequence of SEQ ID NO: 75;
wherein CDR2-L comprises the amino acid sequence of SEQ ID NO: 76;
wherein CDR3-L comprises the amino acid sequence of Z₁-Z₂-W-Z₃-Z₄-Z₅-Z₆-W-Z₇-Z₈;
wherein Z₁ is V, G, P, L, I, M, S, T, or A;
Z₂ is L, G, P, V, I, M, S, T, or A;
Z₃ is Y, F, W, V, L, I, G, or A;
Z₄ is S, G, T, M, N, Q, H, or A;
Z₅ is N, Q, S, T, D, E, H, K, R, or A;
Z₆ is R, S, T, Q, D, E, H, K, N, or A;
Z₇ is V, G, P, L, I, M, S, T, or A; and
Z₈ is F, Y, W, V, L, I, G, or A;
wherein CDR1-H comprises the amino acid sequence of SEQ ID NO: 78;
wherein CDR2-H comprises the amino acid sequence of SEQ ID NO: 79;
wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁; and
wherein Z₉ is V, G, P, L, I, M, S, T, or A;
Z₁₀ is R, S, T, Q, D, E, H, K, N, or A;
Z₁₁ is H, R, K, G, T, S, N, Q, or A;
Z₁₂ is G, P, V, L, I, M, S, T, or A;
Z₁₃ is F, Y, W, V, L, I, G, or A;
Z₁₄ is G, P, V, L, I, M, S, T, or A;
Z₁₅ is N, Q, S, T, D, E, H, K, R, or A;
Z₁₆ is S, G, T, M, N, Q, H, or A;
Z₁₇ is Y, F, W, V, L, I, G, or A;
Z₁₈ is I, G, P, V, L, M, S, T, or A;
Z₁₉ is S, G, T, M, N, Q, H, or A;
Z₂₀ is W, F, Y, V, L, I, G, or A; and
Z₂₁ is Y, F, W, V, L, I, G, or A.

50. The recombinant antibody or antigen binding fragment thereof of claim 49, wherein:
Z₁ is V, G, L, I, or A;
Z₂ is L, V, I, or A;
Z₃ is Y, W, F, or A;
Z₄ is S, G, T, or A;
Z₅ is N, Q, D, E, or A;
Z₆ is R, K, or A;
Z₇ is V, G, L, I, or A;
Z₈ is F, Y, W, or A;
Z₉ is V, G, L, I, or A;
Z₁₀ is R, K, or A;
Z₁₂ is G, S, T, or A;
Z₁₃ is F, Y, W, or A;
Z₁₄ is G, S, T, or A;
Z₁₅ is N, Q, D, E, or A;
Z₁₆ is S, G, T, or A;
Z₁₇ is Y, W, F, or A;
Z₁₈ is I, V, L, or A;
Z₁₉ is S, G, T, or A;
Z₂₀ is W, Y, F, or A; and
Z₂₁ is Y, W, F, or A.

51. The recombinant antibody or antigen binding fragment thereof of claim 49, wherein:
Z₈ is F.

52. The recombinant antibody or antigen binding fragment thereof of claim 49, wherein:
Z₁₀ is R;
Z₁₁ is H;
Z₁₃ is F;
Z₁₈ is I;
Z₁₉ is S; and
Z₂₀ is W.

53. The recombinant antibody or antigen binding fragment thereof of claim 49, wherein CDR3-L comprises an amino acid sequence selected from SEQ ID NOs: 77, 105-106, 108-111, and 113-114.

54. The recombinant antibody or antigen binding fragment thereof of claim 49, wherein the immunoglobulin light chain and the immunoglobulin heavy chain comprise a set of CDR sequences selected from the group consisting of:
CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80; and
CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80.

55. The recombinant antibody or antigen binding fragment thereof of claim 49, wherein the immunoglobulin light chain and the immunoglobulin heavy chain comprise a set of CDR sequences selected from the group consisting of:
CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80; and
CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80.

56. The recombinant antibody or antigen binding fragment thereof of claim 49, wherein CDR3-H comprises an amino acid sequence selected from SEQ ID NOs: 80, 116-119, 121-127, and 129-130.

57. The recombinant antibody or antigen binding fragment thereof of claim 49, wherein the immunoglobulin light chain and the immunoglobulin heavy chain comprise a set of CDR sequences selected from the group consisting of:
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

58. The recombinant antibody or antigen binding fragment thereof of claim 49, wherein the immunoglobulin light chain and the immunoglobulin heavy chain comprise a set of CDR sequences selected from the group consisting of:
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125; and
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

59. The recombinant antibody or antigen binding fragment thereof of claim 47, wherein the CD3 binding domain comprises an immunoglobulin light chain comprising complementarity determining regions (CDRs) CDR1-L, CDR2-L, and CDR3-L, and an immunoglobulin heavy chain comprising CDRs:
CDR1-H, CDR2-H, and CDR3-H, wherein the immunoglobulin light chain and the immunoglobulin heavy chain comprise a set of CDR sequences selected from the group consisting of:
CDR1-L: SEQ ID NO: 75, CDR2-L: SEQ ID NO: 76, CDR3-L: SEQ ID NO: 105, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 106, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 107, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 108, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 109, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 110, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 111, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 112, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 113, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 114, and CDR1-H: SEQ ID NO: 78, CDR2-H: SEQ ID NO: 79, CDR3-H: SEQ ID NO: 80;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 116;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 117;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 118;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 119;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 120;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 121;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 122;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 123;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 124;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 125;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 126;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 127;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 128;
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 129; and
CDR1-L: SEQ ID NO: 75, CDR2-L: SEQ ID NO: 76, CDR3-L: SEQ ID NO: 77, and CDR1-H: SEQ ID NO: 78, CDR2-H: SEQ ID NO: 79, CDR3-H: SEQ ID NO: 130.

60. The recombinant antibody or antigen binding fragment thereof of claim 47, wherein an immunoglobulin light chain of the CD3 binding domain comprises a variable domain of an immunoglobulin kappa (IgK) or immunoglobulin lambda (IgL) light chain.

61. The recombinant antibody or antigen binding fragment thereof of claim 47, wherein an immunoglobulin heavy chain of the CD3 binding domain comprises a variable domain of an IgG1, IgG2, IgG3, or IgG4 heavy chain.

62. The recombinant antibody or antigen binding fragment thereof of claim 47, wherein an immunoglobulin light chain of the CD3 binding domain comprises an amino acid sequence with at least 90%, 95%, 96%, 97%, 98%, 99%, or 100% identity to SEQ ID NO: 81.

63. The recombinant antibody or antigen binding fragment thereof of claim 47, wherein an immunoglobulin heavy chain of the CD3 binding domain comprises an amino acid sequence with at least 90%, 95%, 96%, 97%, 98%, 99%, or 100% identity to SEQ ID NO: 82.

64. The recombinant antibody or antigen binding fragment thereof of claim 47, wherein an immunoglobulin light chain of the CD3 binding domain comprises an amino acid sequence with at least 90%, 95%, 96%, 97%, 98%, 99%, or 100% identity to SEQ ID NO: 81 and an immunoglobulin heavy chain of the CD3 binding domain comprises an amino acid sequence with at least 90%, 95%, 96%, 97%, 98%, 99%, or 100% identity to SEQ ID NO: 82.

65. The recombinant antibody or antigen binding fragment thereof of claim 47, wherein the CD3 binding domain comprises a Fab, Fab′, (Fab′)₂ or a single chain variable fragment (scFv).

66. The recombinant antibody or antigen binding fragment thereof of claim 65, wherein the CD3 binding domain is the scFv.

67. The recombinant antibody or antigen binding fragment thereof of claim 48, wherein the CD3 binding domain comprises an amino acid sequence with at least 90%, 95%, 96%, 97%, 98%, 99%, or 100% identity to SEQ ID NO: 99.

68. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 131.

69. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 132.

70. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 133.

71. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 134.

72. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 135.

73. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 136.

74. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 137.

75. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 138.

76. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 139.

77. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 140.

78. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 141.

79. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 142.

80. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 143.

81. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 144.

82. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 145.

83. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 146.

84. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 147.

85. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 148.

86. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 149.

87. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 150.

88. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 151.

89. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 152.

90. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 153.

91. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 154.

92. The recombinant antibody or antigen binding fragment thereof of claim 49, wherein the CD3 binding domain comprises an amino acid sequence with at least 90%, 95%, 96%, 97%, 98%, 99%, or 100% identity to SEQ ID NO: 155.

93. The recombinant antibody or antigen binding fragment thereof of claim 48, wherein the N-term of the immunoglobulin heavy chain of the TROP2 binding domain is bound to the C-term of the immunoglobulin light chain of the CD3 binding domain.

94. The recombinant antibody or antigen binding fragment thereof of claim 48, wherein the C-term of the immunoglobulin heavy chain of the TROP2 binding domain is bound to the N-term of the immunoglobulin light chain of the CD3 binding domain.

95. The recombinant antibody or antigen binding fragment thereof of claim 48, wherein the N-term of the immunoglobulin heavy chain of the TROP2 binding domain is bound to the C-term of the immunoglobulin heavy chain of the CD3 binding domain.

96. The recombinant antibody or antigen binding fragment thereof of claim 48, wherein the C-term of the immunoglobulin heavy chain of the TROP2 binding domain is bound to the N-term of the immunoglobulin heavy chain of the CD3 binding domain.

97. The recombinant antibody or antigen binding fragment thereof of claim 48, wherein the N-term of the immunoglobulin light chain of the TROP2 binding domain is bound to the C-term of the immunoglobulin light chain of the CD3 binding domain.

98. The recombinant antibody or antigen binding fragment thereof of claim 48, wherein the C-term of the immunoglobulin light chain of the TROP2 binding domain is bound to the N-term of the immunoglobulin light chain of the CD3 binding domain.

99. The recombinant antibody or antigen binding fragment thereof of claim 48, wherein the N-term of the immunoglobulin light chain of the TROP2 binding domain is bound to the C-term of the immunoglobulin heavy chain of the CD3 binding domain.

100. The recombinant antibody or antigen binding fragment thereof of claim 48, wherein the C-term of the immunoglobulin light chain of the TROP2 binding domain is bound to the N-term of the immunoglobulin heavy chain of the CD3 binding domain.

101. The recombinant antibody or antigen binding fragment thereof of claim 48, wherein the CD3 binding domain is a scFv and the TROP2 binding domain is a Fab or Fab′.

102. The recombinant antibody or antigen binding fragment thereof of claim 101, wherein the scFv is bound to the immunoglobulin heavy chain of the Fab or Fab′.

103. The recombinant antibody or antigen binding fragment thereof of claim 101, wherein the scFv is bound to the immunoglobulin light chain of the Fab or Fab′.

104. The recombinant antibody or antigen binding fragment thereof of claim 101, wherein the immunoglobulin light chain of the scFv is bound to the immunoglobulin heavy chain of the Fab or Fab′.

105. The recombinant antibody or antigen binding fragment thereof of claim 101, wherein the immunoglobulin light chain of the scFv is bound to the immunoglobulin light chain of the Fab or Fab′.

106. The recombinant antibody or antigen binding fragment thereof of claim 101, wherein the immunoglobulin heavy chain of the scFv is bound to the immunoglobulin heavy chain of the Fab or Fab′.

107. The recombinant antibody or antigen binding fragment thereof of claim 101, wherein the immunoglobulin heavy chain of the scFv is bound to the immunoglobulin light chain of the Fab or Fab′.

108. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 87 and SEQ ID NO: 88.

109. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 89 and SEQ ID NO: 90.

110. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 91 and SEQ ID NO: 92.

111. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 93 and SEQ ID NO: 94.

112. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 95 and SEQ ID NO: 96.

113. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 97 and SEQ ID NO: 98.

114. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 188.

115. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 189.

116. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 190.

117. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 194.

118. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 195.

119. The recombinant antibody or antigen binding fragment thereof of claim 47, wherein the recombinant antibody or antigen binding fragment thereof comprises amino acid sequences with at least 90%, 95%, 96%, 97%, 98%, 99%, or 100% identity to SEQ ID NO: 158 and SEQ ID NO: 196.

120. The recombinant antibody or antigen binding fragment thereof of claim 47, wherein the recombinant antibody or antigen binding fragment thereof has weaker cytotoxicity activity as compared to a recombinant antibody or antigen binding fragment thereof that comprises an immunoglobulin light chain according to SEQ ID NO: 83 or 85 and an immunoglobulin heavy chain according to SEQ ID NO: 84 or 86 as measured in an in vitro tumor cell killing assay under substantially similar assay conditions.

121. A pharmaceutical composition comprising:
(i) the recombinant antibody or antigen binding fragment thereof of claim 1; and
(ii) a pharmaceutically acceptable excipient.

122. An isolated recombinant nucleic acid molecule encoding a polypeptide of the recombinant antibody or antigen binding fragment thereof of claim 1.

123. A method of treating a cancer in a subject in need thereof comprising administering to the subject the recombinant antibody or antigen binding fragment thereof of claim 1.

124. The method of claim 123, wherein the cancer comprises breast cancer, lung cancer, urothelial cancer, endometrial cancer, ovarian cancer, prostate cancer, pancreatic cancer, gastric cancer, colon cancer, head and neck cancer, or glioma.

125. The method of claim 124, wherein the breast cancer comprises triple-negative breast cancer.

126. The method of claim 124, wherein the lung cancer comprises non-small cell lung cancer.


SEQUENCE LISTING (197 sequences)

SEQ ID NO: 1 - protein, 6 aa, synthetic construct
QDVSIA

SEQ ID NO: 2 - skipped

SEQ ID NO: 3 - protein, 9 aa, synthetic construct
QQHYITPLT

SEQ ID NO: 4 - protein, 9 aa, synthetic construct
AQHYITPLT

SEQ ID NO: 5 - protein, 9 aa, synthetic construct
QAHYITPLT

SEQ ID NO: 6 - protein, 9 aa, synthetic construct
QQAYITPLT

SEQ ID NO: 7 - protein, 9 aa, synthetic construct
QQHAITPLT

SEQ ID NO: 8 - protein, 9 aa, synthetic construct
QQHYATPLT

SEQ ID NO: 9 - protein, 9 aa, synthetic construct
QQHYIAPLT

SEQ ID NO: 10 - protein, 9 aa, synthetic construct
QQHYITALT

SEQ ID NO: 11 - protein, 9 aa, synthetic construct
QQHYITPAT

SEQ ID NO: 12 - protein, 9 aa, synthetic construct
QQHYITPLA

SEQ ID NO: 13 - protein, 8 aa, synthetic construct
GYTFTNYG

SEQ ID NO: 14 - protein, 8 aa, synthetic construct
INTYTGEP

SEQ ID NO: 15 - protein, 14 aa, synthetic construct
ARGGFGSSYWYFDV

SEQ ID NO: 16 - protein, 14 aa, synthetic construct
AAGGFGSSYWYFDV

SEQ ID NO: 17 - protein, 14 aa, synthetic construct
ARAGFGSSYWYFDV

SEQ ID NO: 18 - protein, 14 aa, synthetic construct
ARGAFGSSYWYFDV

SEQ ID NO: 19 - protein, 14 aa, synthetic construct
ARGGAGSSYWYFDV

SEQ ID NO: 20 - protein, 14 aa, synthetic construct
ARGGFASSYWYFDV

SEQ ID NO: 21 - protein, 14 aa, synthetic construct
ARGGFGASYWYFDV

SEQ ID NO: 22 - protein, 14 aa, synthetic construct
ARGGFGSAYWYFDV

SEQ ID NO: 23 - protein, 14 aa, synthetic construct
ARGGFGSSAWYFDV

SEQ ID NO: 24 - protein, 14 aa, synthetic construct
ARGGFGSSYAYFDV

SEQ ID NO: 25 - protein, 14 aa, synthetic construct
ARGGFGSSYWAFDV

SEQ ID NO: 26 - protein, 14 aa, synthetic construct
ARGGFGSSYWYADV

SEQ ID NO: 27 - protein, 14 aa, synthetic construct
ARGGFGSSYWYFAV

SEQ ID NO: 28 - protein, 14 aa, synthetic construct
ARGGFGSSYWYFDA

SEQ ID NO: 29 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCAQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 224 aa, synthetic construct; identical to SEQ ID NO: 30

SEQ ID NO: 33 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQAHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 224 aa, synthetic construct; identical to SEQ ID NO: 30

SEQ ID NO: 35 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQAYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 224 aa, synthetic construct; identical to SEQ ID NO: 30

SEQ ID NO: 37 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHAITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 224 aa, synthetic construct; identical to SEQ ID NO: 30

SEQ ID NO: 39 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYATPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 224 aa, synthetic construct; identical to SEQ ID NO: 30

SEQ ID NO: 41 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYIAPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 224 aa, synthetic construct; identical to SEQ ID NO: 30

SEQ ID NO: 43 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITALTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 224 aa, synthetic construct; identical to SEQ ID NO: 30

SEQ ID NO: 45 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPATFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 224 aa, synthetic construct; identical to SEQ ID NO: 30

SEQ ID NO: 47 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLAFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 224 aa, synthetic construct; identical to SEQ ID NO: 30

SEQ ID NO: 49 - protein, 214 aa, synthetic construct; identical to SEQ ID NO: 29

SEQ ID NO: 50 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCAAGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 214 aa, synthetic construct; identical to SEQ ID NO: 29

SEQ ID NO: 52 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARAGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 214 aa, synthetic construct; identical to SEQ ID NO: 29

SEQ ID NO: 54 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGAFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 214 aa, synthetic construct; identical to SEQ ID NO: 29

SEQ ID NO: 56 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGAGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 214 aa, synthetic construct; identical to SEQ ID NO: 29

SEQ ID NO: 58 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFASSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 214 aa, synthetic construct; identical to SEQ ID NO: 29

SEQ ID NO: 60 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGASYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 214 aa, synthetic construct; identical to SEQ ID NO: 29

SEQ ID NO: 62 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSAYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 63 - protein, 214 aa, synthetic construct; identical to SEQ ID NO: 29

SEQ ID NO: 64 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSAWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 65 - protein, 214 aa, synthetic construct; identical to SEQ ID NO: 29

SEQ ID NO: 66 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYAYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 67 - protein, 214 aa, synthetic construct; identical to SEQ ID NO: 29

SEQ ID NO: 68 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWAFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 69 - protein, 214 aa, synthetic construct; identical to SEQ ID NO: 29

SEQ ID NO: 70 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 71 - protein, 214 aa, synthetic construct; identical to SEQ ID NO: 29

SEQ ID NO: 72 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 73 - protein, 214 aa, synthetic construct; identical to SEQ ID NO: 29

SEQ ID NO: 74 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDAWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 75 - protein, 9 aa, synthetic construct
TGAVTSGNY

SEQ ID NO: 76 - skipped

SEQ ID NO: 77 - protein, 9 aa, synthetic construct
VLWYSNRWV

SEQ ID NO: 78 - protein, 8 aa, synthetic construct
GFTFNKYA

SEQ ID NO: 79 - protein, 10 aa, synthetic construct
IRSKYNNYAT

SEQ ID NO: 80 - protein, 16 aa, synthetic construct
VRHGNFGNSYISYWAY

SEQ ID NO: 81 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 82 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 83 - protein, 214 aa, synthetic construct; identical to SEQ ID NO: 29

SEQ ID NO: 84 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 85 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 224 aa, synthetic construct; identical to SEQ ID NO: 30

SEQ ID NO: 87 - protein, 468 aa, synthetic construct; identical to SEQ ID NO: 85

SEQ ID NO: 88 - protein, 224 aa, synthetic construct; identical to SEQ ID NO: 50

SEQ ID NO: 89 - protein, 468 aa, synthetic construct; identical to SEQ ID NO: 85

SEQ ID NO: 90 - protein, 224 aa, synthetic construct; identical to SEQ ID NO: 70

SEQ ID NO: 91 - protein, 468 aa, synthetic construct; identical to SEQ ID NO: 85

SEQ ID NO: 92 - protein, 224 aa, synthetic construct; identical to SEQ ID NO: 72

SEQ ID NO: 93 - protein, 214 aa, synthetic construct; identical to SEQ ID NO: 29

SEQ ID NO: 94 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCAAGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 95 - protein, 214 aa, synthetic construct; identical to SEQ ID NO: 29

SEQ ID NO: 96 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 97 - protein, 214 aa, synthetic construct; identical to SEQ ID NO: 29

SEQ ID NO: 98 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 99 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 100 - protein, 244 aa, synthetic construct
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 101 - protein, 244 aa, synthetic construct
QDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLT

SEQ ID NO: 102 - protein, 293 aa, synthetic construct
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 103 - protein, 293 aa, synthetic construct
QDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLTAGLIAVIVVVVVALVAGVAVLVISNRRKSGKYKKVEIKELGELRKEPSL

SEQ ID NO: 104 - protein, 9 aa, synthetic construct
TGAVTSANY

SEQ ID NO: 105 - protein, 9 aa, synthetic construct
ALWYSNRWV

SEQ ID NO: 106 - protein, 9 aa, synthetic construct
VAWYSNRWV

SEQ ID NO: 107 - protein, 9 aa, synthetic construct
VLAYSNRWV

SEQ ID NO: 108 - protein, 9 aa, synthetic construct
VLWASNRWV

SEQ ID NO: 109 - protein, 9 aa, synthetic construct
VLWYANRWV

SEQ ID NO: 110 - protein, 9 aa, synthetic construct
VLWYSARWV

SEQ ID NO: 111 - protein, 9 aa, synthetic construct
VLWYSNAWV

SEQ ID NO: 112 - protein, 9 aa, synthetic construct
VLWYSNRAV

SEQ ID NO: 113 - protein, 9 aa, synthetic construct
VLWYSNRWA

SEQ ID NO: 114 - protein, 10 aa, synthetic construct
VLWYSNRWVA

SEQ ID NO: 115 - protein, 8 aa, synthetic construct
GFTFQKYA

SEQ ID NO: 116 - protein, 16 aa, synthetic construct
ARHGNFGNSYISYWAY

SEQ ID NO: 117 - protein, 16 aa, synthetic construct
VAHGNFGNSYISYWAY

SEQ ID NO: 118 - protein, 16 aa, synthetic construct
VRAGNFGNSYISYWAY

SEQ ID NO: 119 - protein, 16 aa, synthetic construct
VRHANFGNSYISYWAY

SEQ ID NO: 120 - protein, 16 aa, synthetic construct
VRHGAFGNSYISYWAY

SEQ ID NO: 121 - protein, 16 aa, synthetic construct
VRHGNAGNSYISYWAY

SEQ ID NO: 122 - protein, 16 aa, synthetic construct
VRHGNFANSYISYWAY

SEQ ID NO: 123 - protein, 16 aa, synthetic construct
VRHGNFGASYISYWAY

SEQ ID NO: 124 - protein, 16 aa, synthetic construct
VRHGNFGNAYISYWAY

SEQ ID NO: 125 - protein, 16 aa, synthetic construct
VRHGNFGNSAISYWAY

SEQ ID NO: 126 - protein, 16 aa, synthetic construct
VRHGNFGNSYASYWAY

SEQ ID NO: 127 - protein, 16 aa, synthetic construct
VRHGNFGNSYIAYWAY

SEQ ID NO: 128 - protein, 16 aa, synthetic construct
VRHGNFGNSYISAWAY

SEQ ID NO: 129 - protein, 16 aa, synthetic construct
VRHGNFGNSYISYAAY

SEQ ID NO: 130 - protein, 16 aa, synthetic construct
VRHGNFGNSYISYWAA

SEQ ID NO: 131 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCARHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 132 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVAHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 133 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 134 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 135 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGAFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 137 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFANSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 138 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGASYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 139 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 140 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSAISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 141 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYASYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 142 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYIAYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 143 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISAWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 144 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYAAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 145 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAAWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 146 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCALWYSNRWVFGGGTKLTVL

SEQ ID NO: 147 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVAWYSNRWVFGGGTKLTVL

SEQ ID NO: 148 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLAYSNRWVFGGGTKLTVL

SEQ ID NO: 149 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWASNRWVFGGGTKLTVL

SEQ ID NO: 150 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYANRWVFGGGTKLTVL

SEQ ID NO: 151 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSARWVFGGGTKLTVL

SEQ ID NO: 152 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNAWVFGGGTKLTVL

SEQ ID NO: 153 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRAVFGGGTKLTVL

SEQ ID NO: 154 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWAFGGGTKLTVL

SEQ ID NO: 155 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVL

SEQ ID NO: 156 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVL

SEQ ID NO: 157 - protein, 249 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVL

SEQ ID NO: 158 - protein, 214 aa, synthetic construct; identical to SEQ ID NO: 29

SEQ ID NO: 159 - protein, 478 aa, synthetic construct; identical to SEQ ID NO: 84

SEQ ID NO: 160 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCARHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 161 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVAHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 162 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 163 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 164 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGAFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 165 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 166 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFANSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 167 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGASYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 168 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 169 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSAISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 170 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYASYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 171 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYIAYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 172 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISAWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 173 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYAAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 174 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAAWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 175 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 176 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVAWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 177 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLAYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 178 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWASNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 179 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYANRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 180 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSARWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 181 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNAWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 182 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRAVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 183 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWAFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 184 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 185 - protein, 478 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 186 - protein, 478 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 187 - protein, 478 aa, synthetic construct; identical to SEQ ID NO: 96

SEQ ID NO: 188 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 189 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 190 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 191 - protein, 478 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 192 - protein, 478 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 193 - protein, 478 aa, synthetic construct; identical to SEQ ID NO: 98

SEQ ID NO: 194 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 195 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 196 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 197 - protein, 478 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC